SEQUENCE LISTING (26 sequences)

SEQ ID NO: 1 - protein, 9 aa, Artificial Sequence
  source: /note="Description of Artificial Sequence: Synthetic peptide"
  VARIANT (1)..(1): /replace="His" or " "
  VARIANT (2)..(2): /replace="Gly" or " "
  VARIANT (3)..(3): /replace="Val" or "Ile" or "Ser" or "Met"
  MOD_RES (5)..(5): Any amino acid
  SITE (1)..(9): /note="Variant residues given in the sequence have no preference with respect to those in the annotations for variant positions"
GDDEXNPGP

SEQ ID NO: 2 - protein, 8 aa, Artificial Sequence
  source: /note="Description of Artificial Sequence: Synthetic peptide"
  VARIANT (2)..(2): /replace="Ile"
  MOD_RES (4)..(4): Any amino acid
  SITE (1)..(8): /note="Variant residues given in the sequence have no preference with respect to those in the annotations for variant positions"
DVEXNPGP

SEQ ID NO: 3 - protein, 9 aa, Artificial Sequence
  source: /note="Description of Artificial Sequence: Synthetic peptide"
GDVESNPGP

SEQ ID NO: 4 - protein, 9 aa, Artificial Sequence
  source: /note="Description of Artificial Sequence: Synthetic peptide"
GDIEENPGP

SEQ ID NO: 5 - protein, 7 aa, Artificial Sequence
  source: /note="Description of Artificial Sequence: Synthetic peptide"
VEPNPGP

SEQ ID NO: 6 - protein, 7 aa, Artificial Sequence
  source: /note="Description of Artificial Sequence: Synthetic peptide"
IETNPGP

SEQ ID NO: 7 - protein, 9 aa, Artificial Sequence
  source: /note="Description of Artificial Sequence: Synthetic peptide"
GDIESNPGP

SEQ ID NO: 8 - protein, 9 aa, Artificial Sequence
  source: /note="Description of Artificial Sequence: Synthetic peptide"
GDVELNPGP

SEQ ID NO: 9 - protein, 9 aa, Artificial Sequence
  source: /note="Description of Artificial Sequence: Synthetic peptide"
GDIETNPGP

SEQ ID NO: 10 - protein, 8 aa, Artificial Sequence
  source: /note="Description of Artificial Sequence: Synthetic peptide"
GDVENPGP

SEQ ID NO: 11 - protein, 9 aa, Artificial Sequence
  source: /note="Description of Artificial Sequence: Synthetic peptide"
GDVEENPGP

SEQ ID NO: 12 - protein, 9 aa, Artificial Sequence
  source: /note="Description of Artificial Sequence: Synthetic peptide"
GDVEQNPGP

SEQ ID NO: 13 - protein, 7 aa, Artificial Sequence
  source: /note="Description of Artificial Sequence: Synthetic peptide"
IESNPGP

SEQ ID NO: 14 - protein, 9 aa, Artificial Sequence
  source: /note="Description of Artificial Sequence: Synthetic peptide"
GDIELNPGP

SEQ ID NO: 15 - protein, 9 aa, Artificial Sequence
  source: /note="Description of Artificial Sequence: Synthetic peptide"
HDIETNPGP

SEQ ID NO: 16 - protein, 9 aa, Artificial Sequence
  source: /note="Description of Artificial Sequence: Synthetic peptide"
HDVETNPGP

SEQ ID NO: 17 - protein, 9 aa, Artificial Sequence
  source: /note="Description of Artificial Sequence: Synthetic peptide"
HDVEMNPGP

SEQ ID NO: 18 - protein, 9 aa, Artificial Sequence
  source: /note="Description of Artificial Sequence: Synthetic peptide"
GDMESNPGP

SEQ ID NO: 19 - protein, 9 aa, Artificial Sequence
  source: /note="Description of Artificial Sequence: Synthetic peptide"
GDVETNPGP

SEQ ID NO: 20 - protein, 9 aa, Artificial Sequence
  source: /note="Description of Artificial Sequence: Synthetic peptide"
GDIEQNPGP

SEQ ID NO: 21 - protein, 8 aa, Artificial Sequence
  source: /note="Description of Artificial Sequence: Synthetic peptide"
DSEFNPGP

SEQ ID NO: 22 - RNA, 90 nt, Artificial Sequence
  source: /note="Description of Artificial Sequence: Synthetic oligonucleotide"
cgggucggcauggcaucuccaccuccucgcgguccgaccugggcauccgaaggaggacgcacguccacucggauggcuaagggagagcca

SEQ ID NO: 23 - RNA, 88 nt, Artificial Sequence
  source: /note="Description of Artificial Sequence: Synthetic oligonucleotide"
uggccggcauggucccagccuccucgcuggcgccggcugggcaacauuccgaggggaccguccccucgguaauggcgaaugggaccca

SEQ ID NO: 24 - RNA, 51 nt, Artificial Sequence
  source: /note="Description of Artificial Sequence: Synthetic oligonucleotide"
ucauaaucaauuuauuauuuucuuuuauuuuauucacauaauuuuguuuuu

SEQ ID NO: 25 - RNA, 19 nt, Artificial Sequence
  source: /note="Description of Artificial Sequence: Synthetic oligonucleotide"
auuuuguuuuuaacauuuc

SEQ ID NO: 26 - RNA, 70 nt, Artificial Sequence
  source: /note="Description of Artificial Sequence: Synthetic oligonucleotide"
ucauaaucaauuuauuauuuucuuuuauuuuauucacauaauuuuguuuuuauuuuguuuuuaacauuuc

(19) United States
(12) Patent Application Publication
(10) Pub. No.: US 2023/0340451 A1
(43) Pub. Date: Oct. 26, 2023

(54) COMPOSITIONS COMPRISING LINEAR POLYRIBONUCLEOTIDES FOR PROTEIN MODULATION AND USES THEREOF

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US)

(21) Appl. No.: 17/795,939

(22) PCT Filed: Jan. 29, 2021
(86) PCT No.: PCT/US2021/015746

§ 371 Date: Jul. 28, 2022

Related U.S. Application Data
(60) Provisional application No. 62/967,544, filed on Jan. 29, 2020.

(51) Int. Cl. C12N 9/10; C12N 11/06; C12N 15/115

(52) U.S. Cl. CPC C12N 11/06; C12N 9/104; C12N 15/115; C12N 2310/16; C12N 2310/351

(57) ABSTRACT

This invention relates to compositions comprising linear polyribonucleotides and uses thereof.

### SEQUENCE LISTING

The instant application contains a Sequence Listing which has been submitted electronically in ASCII format and is hereby incorporated by reference in its entirety. Said ASCII copy, created on Jul. 21, 2022, is named 51509-016002_Sequence_Listing_7_21_22_ST25 and is 7,790 bytes in size.

### CROSS-REFERENCE TO RELATED APPLICATIONS

The present application claims priority to and benefit from U.S. Provisional Application No. 62/967,544, filed Jan. 29, 2020, the entire contents of which is herein incorporated by reference.

### BACKGROUND

Certain linear polyribonucleotides are ubiquitously present in human tissues and cells, including tissues and cells of healthy individuals.

### SUMMARY

This disclosure relates generally to compositions, pharmaceutical compositions, and preparations of linear polyribonucleotides and uses thereof for protein modulation. The linear polyribonucleotides of the disclosure can be used for modulation of a substrate protein. The compositions include, and the methods use, linear polyribonucleotide comprising two conjugation moieties for conjugation to a chemical compound, such as small molecules, each of which binds a target protein or a substrate protein for degradation of the substrate protein. The compositions include, and the methods use, linear polyribonucleotide comprising a conjugation moiety for conjugation to a chemical compound, such as a small molecule, that binds a target protein and a binding site that binds a substrate protein, for degradation of the substrate protein. The compositions include, and the methods use, linear polyribonucleotide comprising a binding site that binds a target protein and a conjugation moiety for conjugation to a chemical compound, such as a small molecule, that binds a binding site that binds a substrate protein, for degradation of the substrate protein. The target protein can be a ubiquitin ligase that ubiquitinates the substrate protein, resulting in degradation of the substrate protein. The substrate protein for degradation can be a pathogenic protein.

In a first aspect, the invention features a composition comprising a linear polyribonucleotide comprising a first conjugation moiety and a second conjugation moiety, wherein the first conjugation moiety conjugates the linear polyribonucleotide to a first chemical compound (e.g., a small molecule) that binds to a target protein that modulates a substrate protein and wherein the second conjugation moiety conjugates the linear polyribonucleotide to a second chemical compound that binds the substrate protein.

In a second aspect, the invention features a composition comprising:a) linear polyribonucleotide comprising a first conjugation moiety and a second conjugation moiety, b) a first chemical compound that binds a target protein; and c) a second chemical compound that binds a substrate protein; wherein the linear polyribonucleotide is conjugated to the first chemical compound by the first conjugation moiety, the linear polyribonucleotide is conjugated to the second chemical compound by the second conjugation moiety, and the target protein modulates the substrate protein.

In a third aspect, the invention features a composition comprising a linear polyribonucleotide comprising a conjugation moiety and a binding site conjugation moiety, wherein the conjugation moiety conjugates the linear polyribonucleotide to a chemical compound (e.g., a small molecule) and wherein the binding site binds to a protein.

In a fourth aspect, the invention features a composition comprising: a) linear polyribonucleotide comprising a conjugation moiety and a binding site; and b) a chemical compound; wherein the linear polyribonucleotide is conjugated to the chemical compound by the conjugation moiety, and i) the chemical compound binds a target protein and the binding site binds substrate protein; or ii) the chemical compound binds the substrate protein and the binding site binds the target protein.

In some embodiments, the binding site is an aptamer. In some embodiments, the binding site is a miRNA binding site. In some embodiments, the conjugation moiety is a modified nucleotide. In some embodiments, the first conjugation moiety is a first modified nucleotide and the second conjugation moiety is a second modified nucleotide. In some embodiments, the first modified nucleotide and second modified nucleotide are the same. In some embodiments, the first modified nucleotide and second modified nucleotide are different. In some embodiments, the modified nucleotide, the first modified nucleotide, or the second modified nucleotide is a modified UTP analog, a modified ATP analog, modified CTP analog, or a modified GTP analog. In some embodiments, the modified UTP analog is a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG. In some embodiments, the modified nucleotide, the first modified nucleotide, or the second modified nucleotide comprises a click chemistry moiety. In some embodiments, the the first chemical compound is a small molecule. In some embodiments, the chemical compound or the first chemical compound recruits or binds the target protein. In some embodiments, the chemical compound or the first chemical compound is a target protein ligand. In some embodiments, the chemical compound or the first chemical compound is an LCL161 derivative, VHL-1, pomalidomide, lenalidomide, thalidomide or a derivative thereof, a HIF-1a-derived (R)-hydroxyproline, VHL ligand 2, VL-269, a VH032 derivative, or a hydroxyproline-based ligand. In some embodiments, the chemical compound or the second chemical compound is a small molecule. In some embodiments, the chemical compound or the second chemical compound binds to a misfolded protein. In some embodiments, the chemical compound or the second chemical compound binds to a disease-associated protein. In some embodiments, the chemical compound or the second chemical compound binds to a protein associated with cancer. In some embodiments, the chemical compound or the second chemical compound is a Heat Shock Protein 90 (HSP90) inhibitor, Kinase and Phosphatase inhibitor, MDM2 inhibitor, HDAC inhibitor, Human Lysine Methyltransferase Inhibitor, Angiogenesis inhibitor, or Immunosuppressive compound. In some embodiments, the chemical compound or the second chemical compound binds to a Human BET Bromodomain-containing protein, the aryl hydrocarbon receptor (AHR), REF receptor kinase, FKBP, Androgen Receptor (AR), Estrogen receptor (ER), Thyroid Hormone Receptor, HIV Protease, HIV Integrase, HCV Protease, Acyl-protein Thioesterase-1 and-2 (APTI and APT2); BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, or MDM2. In some embodiments, the chemical compound or the second chemical compound is dasatinib, lapatinib, gefitinib, foretinib, Sirt2 inhibitor 3b, Sirt2 inhibitor, SNS-032, AC220, ceritinib, ibrutinib, ibrutinib dertivatie, 4-OHT, Jq1, PDE4 inhibitor, thiazolidinedione-based ligand, ripk2 inhibitor, bosutinib, OTX015, steel factor, TBK1 inhibitor, HJB97, aminopyrazole analog, RN486, AR antagonist, IACS-73, or nutlin small molecule. In some embodiments, the target protein is an enzyme. In some embodiments, the enzyme is a post-translational modification enzyme. In some embodiments, the target protein modifies the substrate by adding a functional group to the substrate protein. In some embodiments, the target protein modifies a substrate protein by adding a functional group to the substrate protein. In some embodiments, the modification is by acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, famesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, succinylation, sulfation, S-sulfenylation, S-sulfinylation, succinylation, sumoylation, ubiquitination, or uridylylation. In some embodiments, the target protein is a ubiquitin ligase. In some embodiments, the ubiquitin ligase is a HECT, RING-finger, U-box, or PHD-finger ubiquitin ligase. In some embodiments, the ubiquitin ligase is selected from the group consisting of von Rippel-Lindau (VHL); cereblon; XTAP; E3A; MDM2; Anaphase-promoting complex (APC); UBR5 (EDDI); SOCS/ BC-box/ eloBC/ CUL5/ RING; LNXp80; CBX4; CBLLI; HACEI; HECTDI; HECTD2; HECTD3; HECWI; HECW2; HERCI; HERC2; HERC3; HERC4; HUWEI; ITCH; NEDD4; NEDD4L; PPIL2; PRPF19; PIASI; PIAS2; PIAS3; PIAS4; RANBP2; RNF4; RBXI; SMURFI; SMURF2; STUBI; TOPORS; TRIP12; UBE3A; UBE3B; UBE3C; UBE4A; UBE4B; UBOX5; UBR5; WWPI; WWP2; Parkin; A20/TNFAIP3; AMFR/gp78; ARA54; beta-TrCPl/BTRC; BRCAI; CBL; CHIP/STUB I; E6; E6AP/UBE3A; F-box protein 15/FBXOIS; FBXW7/Cdc4; GRAIL/RNF128; HOIP/RNF3 1; cIAP-⅟HIAP-2; cIAP- 2/HIAP-1; cIAP (pan); ITCH/AIP4; KAPI; MARCH8;; Mind Bomb ⅟MIBI; Mind Bomb 2/MIB2; MuRF1/TRIM63; NDFIPI; NEDD4; NleL; Parkin; RNF2; RNF4; RNF8; RNF168; RNF43; SARTI; Skp2; SMURF2; TRAF-1; TRAF-2; TRAF-3; TRAF-4; TRAF-5; TRAF-6; TRIMS; TRIM21; TRIM32; UBR5; and ZNRF3. In some embodiments, the substrate protein is a disease-associated protein. In some embodiments, the substrate protein is a misfolded protein. In some embodiments, the substrate protein comprises a mutation as compared to a wild-type version of the substrate protein. In some embodiments, the substrate protein is BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, MDM2, FoxOl, HDAC, DP-1, E2F, ABL, ALK, AMPK, BRK, BRSK I, BRSK2, BTK, CAMKKI, CAMKK alpha, CAMKK beta, Rb, Suv39HI, SCF, pl9INK4D, GSK-3, pi 8 INK4, myc, cyclin E, CDK2, CDK9, CDG4/6, Cycline D, pl6 INK4A, cdc25A, BMII, SCF, Akt, CHK½, CI delta, CKI gamma, C 2, CLK2, CSK, DDR2, DYRKIA/2/3, EF2K, EPH-A2/A4/B1/B2/B3/B4, EIF2A 3, Smad2, Smad3, Smad4, Smad7, p53, p21 Cipl, PAX, Fyn, CAS, C3G, SOS, Tal, Raptor, RACK-I, CRK, Rapl, Rae, KRas, NRas, HRas, GRB2, FAK, PBK, spred, Spry, mTOR, MPK, LKBl, PAK 1/2/4/5/6, PDGFRA, PYK.2, Src, SRPKI, PLC, PKC, PKA, PKB, alpha/beta, PKC alpha/gamma/zeta, PKD, PLK1, PRAK, PRK2, RIPK2, WA VE-2, TSC2, DAPK1, BAD, IMP, C-TAKI, TAKI, TAO1, TBKI, TESKI, TGFBRI, TIE2, TLKI, TrkA, TSSKI, TIBKI/2, TTK, Tp12/cot1, MEKI, MEK2, PLDL Erk1, Erk2, Erk5, Erk8, p90RSK, PEA- 15, SRF, p27 KIPI, TIF 1a, HMGNI, ER81, MKP-3, c-Fos, FGF-R1, GCK, GSK3 beta, HER4, HIPKI/2/3/, IGF-IR, cdc25, UBF, LAMTOR2, Statl, StaO, CREB, JAK, Src, SNCA, PTEN, NF- kappaB, HECTH9, Bax, HSP70, HSP90, Apaf-1, Cyto c, BCL-2, Bcl-xL, BCL-6, Smac, XIAP, Caspase-9, Caspase-3, Caspase-6, Caspase-7, CDC37, TAB, IKK, TRADD, TRAF2, RIPI, FLIP, TAKI, JNK1/2/3, Lek, A-Raf, B-Raf, C-Raf, MOS, MLK1/3 MN 1/2, MSK1, MST2/3/4, MPSKI, MEKK1, ME K4, MEL, ASKI, MINK I, MKK 1/2/2/4/6/7, NE, 2a/6/7, NUAKI, OSRI, SAP, STK33, Syk, Lyn, PDKI, PHK, PIM 1/2/3, Ataxin- 1, mTORCl, MDM2, p21 Wafl, Cyclin D1, Lamln A, Tp12, Myc, catenin, Wnt, IKK-beta, IKKgamma, IKK-alpha, IKK-epsilon, ELK, p65Re1A, IRAKI, IRA 2, IRAK4, IRR, FADD, TRAF6, TRAF3, MKK3, MKK6, ROCK2, RSKI/2, SGK 1, SmMLCK, SIK⅔, ULKI/2, VEGFRI, WNK 1, YESI, ZAP70, MAP4K3, MAP4K5, MAPKlb, MAPKAP-K2 K3, p38, alpha/beta/delta/gamma MAPK, Aurora A, Aurora B, Aurora C, MCAK, Clip, MAPKAPK, FAK, MARK 1 /2/3/4, Mucl, SHC, CXCR4, Gap-I, Myc, beta-catenin/TCF, Cbl, BRM, Mell, BRD2, BRD3, BRD4, AR, RAS, ErbB3, EGFR, IREI, HPKI, RIPK2, ERa, or PCAF/GCN5. In some embodiments, the composition further comprises the target protein and/or the substrate protein; and optionally, forms a complex. In some embodiments, the complex alters substrate protein interactions with other proteins. In some embodiments, the complex increases activity of the substrate protein. In some embodiments, the complex decreases activity of the substrate protein. In some embodiments, the complex alters localization of the substrate protein. In some embodiments, the complex alters stability of the substrate protein. In some embodiments, the complex promotes degradation of the substrate protein. In some embodiments, the degradation of the substrate protein comprises proteasomal degradation. In some embodiments, the complex promotes ubiquitination of the substrate protein. In some embodiments, the linear polyribonucleotide is an exogenous, synthetic linear polyribonucleotide. In some embodiments, the linear polyribonucleotide lacks a poly-A sequence, lacks a replication element, is translation incompetent, or any combination thereof.

In a fifth aspect, the invention features a pharmaceutical composition comprising the composition of any one of the previous embodiments and a pharmaceutically acceptable carrier or excipient.

In a sixth aspect, the invention features a pharmaceutical composition comprising the composition of any one of the previous embodiments and a pharmaceutically acceptable excipient and is free of any carrier.

In some embodiments, the composition of any one of the previous embodiments is for use as a medicament or a pharmaceutical. In some embodiments, the composition of any one of the previous embodiments, or the pharmaceutical composition of the previous embodiments is for use in a method of treatment of a human or animal body. In some embodiments, the composition of any one of the previous embodiments, or the pharmaceutical composition of the previous embodiments is formulated for intravenous administration or intratumoral administration. In some embodiments, the composition of any one of the previous embodiments, or the pharmaceutical composition of the previous embodiments is for use in a method of treating a cancer or a hyperproliferative disease; a neurodegenerative disease; a metabolic disorder; an inflammatory disorder; an autoimmune disease; an infectious disease; or a genetic disease. In some embodiments, the composition of any one of the previous embodiments, or the pharmaceutical composition of the previous embodiments is for use in a method of treating a solid tumor (e.g., a reproductive tissue cancer, e.g., cervical cancer or prostate cancer) or a liquid tumor (e.g., lymphoma, e.g., a B cell lymphoma).

In a seventh aspect, the invention features a use of the composition of any one of the previous embodiments in the manufacture of a medicament or a pharmaceutical.

In an eighth aspect, the invention features a use of the composition of any one of the previous embodiments in the manufacture of a medicament or a pharmaceutical for treating a human or animal body by therapy.

In a ninth aspect, the invention features a use of the composition of any one of the previous embodiments in the manufacture of a medicament for treating a cancer or a hyperproliferative disease; a neurodegenerative disease; a metabolic disorder; an inflammatory disorder; an autoimmune disease; an infectious disease; or a genetic disease.

In a tenth aspect, the invention features a use of the composition of any one of the previous embodiments in the manufacture of a medicament for treating a solid tumor (e.g., a reproductive tissue cancer, e.g., cervical cancer or prostate cancer) or a liquid tumor (e.g., lymphoma, e.g., a B cell lymphoma).

### Definitions

As used herein, the terms “linear RNA” or “linear polyribonucleotide” or “linear polyribonucleotide molecule” are used interchangeably and mean a monoribonucleotide molecule or polyribonucleotide molecule having a 5′ and 3′ end. In some embodiments, the linear RNA has a free 5′ end or a free 3′ end. In some embodiments, the linear RNA has a 5′ end or 3′ end that is modified or protected from degradation (e.g., by a 5′ end protectant or a 3′ end protectant).

As used herein, the term “aptamer sequence” is a non-naturally occurring or synthetic oligonucleotide that specifically binds to a target molecule. Typically an aptamer is from 20 to 500 nucleotides. Typically an aptamer binds to its target through secondary structure rather than sequence homology.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the term “immunoprotein binding site” is a nucleotide sequence that binds to an immunoprotein. In some embodiments, the immunoprotein binding site aids in masking the linear polyribonucleotide as exogenous, for example, the immunoprotein binding site is bound by a protein (e.g., a competitive inhibitor) that prevents the linear polyribonucleotide from being recognized and bound by an immunoprotein, thereby reducing or avoiding an immune response against the linear polyribonucleotide..

As used herein, the term “modified ribonucleotide” means any ribonucleotide analog or derivative that has one or more chemical modifications to the chemical composition of an unmodified natural ribonucleotide, such as a natural unmodified nucleotide adenosine (A), uridine (U), guanine (G), cytidine (C). In some embodiments, the chemical modifications of the modified ribonucleotide are modifications to any one or more functional groups of the ribonucleotide, such as, the sugar the nucleobase, or the internucleoside linkage (e.g. to a linking phosphate / to a phosphodiester linkage / to the phosphodiester backbone).

As used herein, the phrase “quasi-helical structure” is a higher order structure of the linear polyribonucleotide, wherein at least a portion of the linear polyribonucleotide folds into a helical structure.

As used herein, the phrase “quasi-double-stranded secondary structure” is a higher order structure of the linear polyribonucleotide, wherein at least a portion of the linear polyribonucleotide creates a double strand.

As used herein, the term “regulatory sequence” is a nucleic acid sequence that modifies an expression product.

As used herein, the term “repetitive nucleotide sequence” is a repetitive nucleic acid sequence within a stretch of DNA or throughout a genome. In some embodiments, the repetitive nucleotide sequence includes poly CA or poly TG sequences. In some embodiments, the repetitive nucleotide sequence includes repeated sequences in the Alu family of introns.

As used herein, the term “replication element” is a sequence and/or motifs useful for replication or that initiate transcription of the linear polyribonucleotide.

As used herein, the term “selective translation sequence” is a nucleic acid sequence that selectively initiates or activates translation of an expression sequence in the linear polyribonucleotide.

As used herein, the term “selective degradation sequence” is a nucleic acid sequence that initiates degradation of the circular polyribonucleotide, or an expression product of the linear polyribonucleotide.

As used herein, the term “stagger sequence” is a nucleotide sequence that induces ribosomal pausing during translation. In some embodiments, the stagger sequence is a non-conserved sequence of amino-acids with a strong alpha-helical propensity followed by the consensus sequence -D(V/I)ExNPG P, where x is any amino acid.

As used herein, the term “substantially resistant” is one that has at least 50%, 60%, 70%, 75%, 80%, 85%, 90%, 95%, 96%, 97%, 98% or 99% resistance as compared to a reference.

As used herein, the term “complex” means an association between at least two moieties (e.g. chemical or biochemical) that have an affinity for one another.

“Polypeptide” and “protein” are used interchangeably and means a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics).

As used herein, the term “binding site” is a region of the linear polyribonucleotide that interacts with another entity, e.g., a chemical compound, a protein, a nucleic acid, etc.

As used herein, the term “binding moiety” is a region of a target that can be bound by a binding site, for example, a region, domain, fragment, epitope, or portion of a nucleic acid (e.g., RNA, DNA, RNA-DNA hybrid), chemical compound, small molecule (e.g., drug), aptamer, polypeptide, protein, lipid, carbohydrate, antibody, virus, virus particle, membrane, multi-component complex, organelle, cell, other cellular moieties, any fragment thereof, and any combination thereof.

As used herein, the term “conjugation moiety” means a modified nucleotide comprising a functional group for use in a method of conjugation.

As used herein, the term “carrier” means a compound, composition, reagent, or molecule that facilitates the transport or delivery of a composition (e.g., a linear polyribonucleotide) into a cell by a covalent modification of the linear polyribonucleotide, via a partially or completely encapsulating agent, or a combination thereof. Non-limiting examples of carriers include carbohydrate carriers (e.g., an anhydride- modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the linear polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a linear polyribonucleotide is a formulation that comprises a linear polyribonucleotide without covalent modification and is free from a carrier.

The term “diluent” means vehicle comprising an inactive solvent in which a composition described herein (e.g., a composition comprising a linear polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3- butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “parenterally acceptable diluent” is a diluent used for parenteral administration of a composition (e.g., a composition comprising a linear polyribonucleotide).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 provides a schematic of a linear RNA that binds an enzyme (Enz) and a substrate (substrate), thereby bringing the two into close proximity and promoting modification (M) of the substrate by the enzyme.

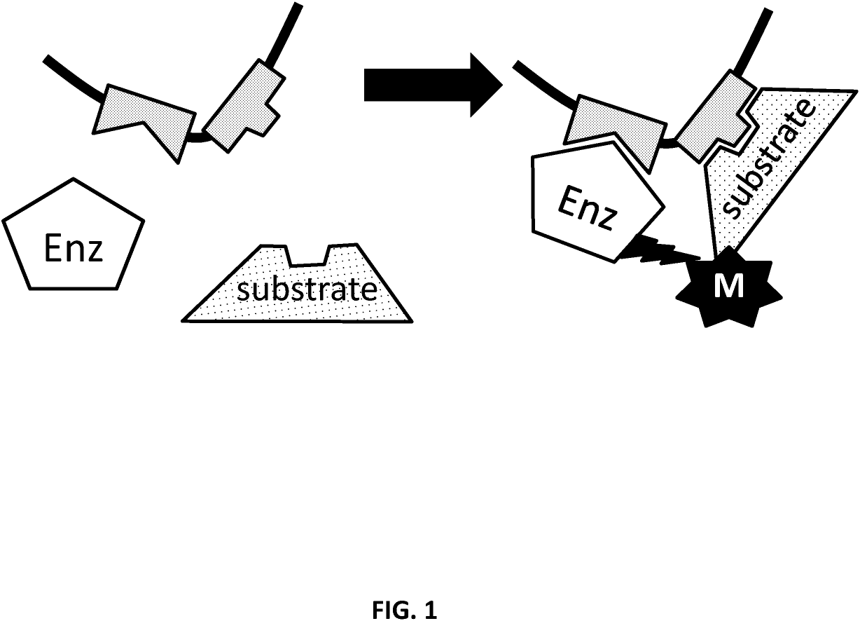

FIG. 2 illustrates click chemistry conjugation of a chemical compound (e.g., a small molecule) to a linear RNA to generate a linear RNA that can bind an E3 ubiquitin ligase.

FIG. 3 illustrates a linear RNA containing a protein binding sequence and a conjugated small molecule that can recruit an E3 ubiquitin ligase and a second protein, and target the second protein for ubiquitination and degradation.

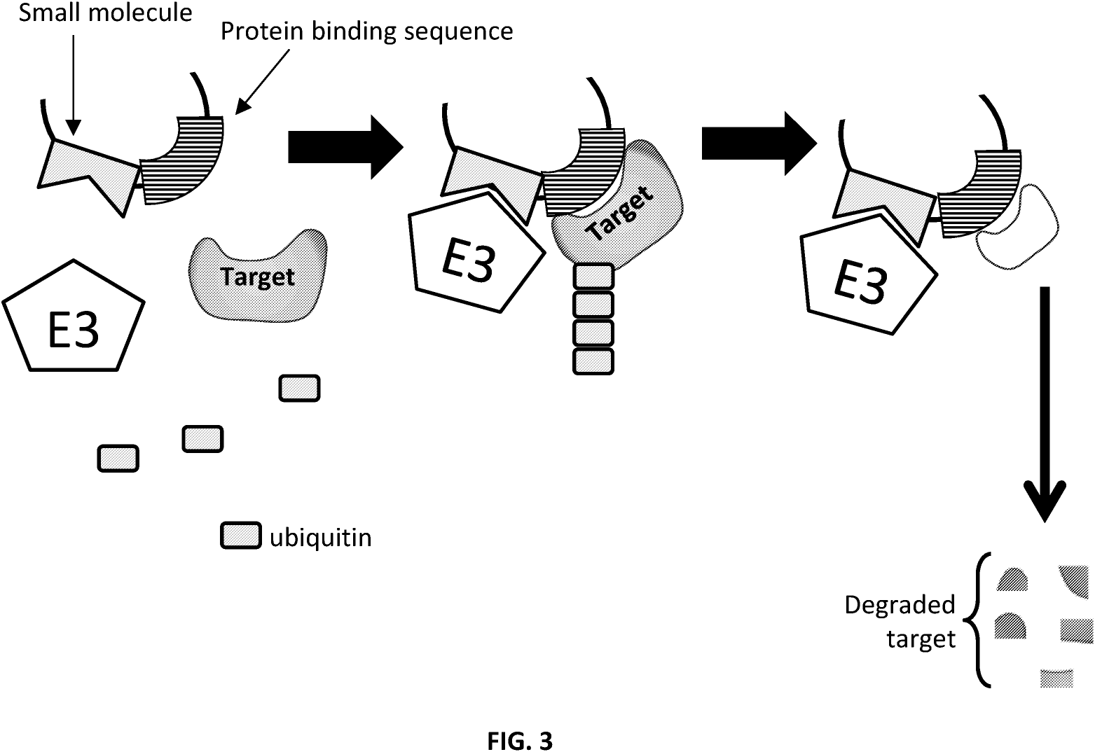

FIG. 4 provides a schematic for generation of a linear RNA comprising two small molecules.

FIG. 5 illustrates a linear RNA comprising two conjugated small molecule that can target a protein for ubiquitination and degradation.

FIG. 6 illustrates a linear RNA that can bind two proteins and target one of the proteins for ubiquitination and degradation.

### DETAILED DESCRIPTION

This disclosure relates generally to compositions, pharmaceutical compositions, and preparations of linear polyribonucleotides and uses thereof for protein modulation. The linear polyribonucleotides of the disclosure can be used for modulation of a substrate protein. In some embodiments, linear polyribonucleotides of the disclosure form complexes with target proteins that modify substrate proteins. For example, a linear polyribonucleotide can comprise a conjugation moiety, wherein the conjugation moiety conjugates the linear polyribonucleotide to a chemical compound (e.g., a small molecule). This chemical compound can bind to a target protein, wherein the target protein modulates a substrate protein. The linear polyribonucleotide can further comprise a binding site that binds to the substrate protein or can further comprise a second conjugation moiety that is conjugated to a second chemical compound that binds to the substrate protein. Therefore, the target protein and the substrate protein can be localized to the linear polyribonucleotide, allowing for the target protein to modulate the substrate protein.

### Linear Polyribonucleotides

Linear polyribonucleotides described herein are a polyribonucleotide molecule having a 5′ and 3′ end. In some embodiments, the linear RNA has a free 5′ end or a free 3′ end. In some embodiments, the linear RNA has a 5′ end or 3′ end that is modified or protected from degradation. In some embodiments, the linear RNA has non-covalently linked 5′ or 3′ ends.

The present disclosure includes compositions comprising synthetic RNA and methods of their use. The linear polyribonucleotides of the disclosure can be used for modulation of a substrate protein. Due to the linear structure, linear RNA can be modified at its ends to improve stability and/or reduce degradation. For example, the 5′ free end and/or 3′ free end comprises a cap, a poly-A tail, a G-quadruplex, a pseudoknot, a stable terminal stem loop, U-rich expression, a nuclear retention element (ENE), or a conjugation moiety. For example, the 5′ free end and/or 3′ free end comprises an end protectant, such as a cap, a poly-A tail, a g-quadruplex, a pseudoknot, a stable terminal stem loop, U-rich expression, a nuclear retention element (ENE), or a conjugation moiety.

In some embodiments, a linear RNA binds a target. In some embodiments, a linear RNA binds a target and binds a substrate of the target. In some embodiments, a linear RNA binds a target and mediates modulation of a substrate of the target. In some embodiments, a linear RNA brings together a target and its substrate to mediate modification of the substrate, e.g., post-translational modification. In some embodiments, a linear RNA brings together a target and its substrate to mediate a cellular process (e.g., alters protein degradation or signal transduction) involving the substrate. In some embodiments, a target is a target protein and a substrate is a substrate protein.

In some embodiments, the linear polyribonucleotide as describe herein is a bifunctional linear polyribonucleotide. In some embodiments, a bifunctional linear polyribonucleotide herein has the following structure:

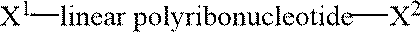

wherein X¹ and X² independently comprises a molecule (e.g., a chemical compound or a binding site) comprising an E3 ubiquitin ligase binding moiety (UBM) or a molecule (e.g., a chemical compound or a binding site) comprising a protein binding moiety (PBM), or a combination thereof. For example, in some embodiments, X¹ comprises an UBM, and X² comprises a PBM. In some embodiments, X¹ and X² each independently comprise one or more UBMs and one or more PBMs. In some embodiments, each X¹ and X² independently comprises at least about 2, 3, 4, 5, 10, 15, 20, 25, 30, 50, or 100 UBMs and PBMs. In some embodiments, each X¹ and X² independently comprises at most about 2, 3, 4, 5, 10, 15, 20, 25, 30, 50, or 100 UBMs and PBMs. Such two more binding moieties (e.g., UBMs and/or PBMs) can be coupled to each other in a linear or in a branched fashion. An example for a linear configuration of three identical or different UBMs is: UBM¹-UBM²-UBM³ . An example for a branched configuration of four identical or different UBMs is:

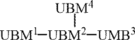

A UBM herein can be any molecule capable of binding (e.g., covalently or non-covalently) an E3 ubiquitin ligase (e.g., a target protein) as described herein. Such E3 ubiquitin ligase can include Rippel-Lindau (VHL); cereblon; XIAP; E3A; MDM2; Anaphase-promoting complex (APC); UBR5 (EDDI); SOCS/BC-box/ eloBC/ CUL5/ RING; LNXp80; CBX4; CBLLI; HACEI; HECTDI; HECTD2; HECTD3; HECWI; HECW2; HERCI; HERC2; HERC3; HERC4; HUWEI; ITCH; NEDD4; NEDD4L; PPIL2; PRPF19; PIASI; PIAS2; PIAS3; PIAS4; RANBP2; RNF4; RBXI; SMURFI; SMURF2; STUBI; TOPORS; TRIP12; UBE3A; UBE3B; UBE3C; UBE4A; UBE4B; UBOX5; UBR5; WWPI; WWP2; Parkin; A20/TNFAIP3; AMFR/gp78; ARA54; beta-TrCPl/BTRC; BRCAI; CBL; CHIP/STUB I; E6; E6AP/UBE3A; F-box protein 15/FBXOIS; FBXW7/Cdc4; GRAIL/RNF128; HOIP/RNF3 1; cIAP-⅟HIAP-2; cIAP-2/HIAP-1; cIAP (pan); ITCH/AIP4; KAPI; MARCH8; Mind Bomb ⅟MIBI; Mind Bomb 2/MIB2; MuRFl/TRIM63; NDFIPI; NEDD4; NleL; Parkin; RNF2; RNF4; RNF8; RNF168; RNF43; SARTI; Skp2; SMURF2; TRAF-1; TRAF-2; TRAF-3; TRAF-4·; TRAF-5; TRAF-6; TRIMS; TRIM21; TRIM32; UBR5; and ZNRF3. Further such examples of E3 ligases include those from Tables 13-27 in EP3458101, which is hereby incorporated by reference in its entirety.

A PBM herein can be any molecule capable of binding (e.g., covalently or non-covalently) a protein (e.g., a target protein) as described herein. Examples of proteins that a PBM herein bind to include a Von Hippel-Lindau E3 ubiquitin ligase, a cereblon E3 ubiquitin ligase, an MDM2 E3 ubiquitin ligase binding moiety, or an inhibitor of apoptosis (IAP).

In some embodiments, a UBM or a PBM binds to single protein, e.g, a ligase. In other embodiments, a UBM or a PBM herein is configured to bind to 2 or more identical or different proteins. Such binding to multiple proteins can occur either simultaneously or sequentially. Further examples of proteins include, but are not limited to, E3 ligases from Tables 13-27 in EP3458101, which is hereby incorporated by reference in its entirety.

In some embodiments, a linear RNA comprises a conjugation moiety for binding to a chemical compound. The conjugation moiety can be a modified polyribonucleotide. The conjugation moiety can be at any polyribonucleotide in the linear RNA. The chemical compound can be conjugated to the linear polyribonucleotide by the conjugation moiety. In some embodiments, the chemical compound binds to a target and mediates modulation of a substrate of the target. In some embodiments, a linear RNA binds a substrate of a target and a chemical compound conjugated to the linear RNA by the conjugation moiety binds the target to bring together the target and its substrate to mediate modification of the substrate, e.g., post-translational modification. In some embodiments, a linear RNA binds a substrate of a target and a chemical compound conjugated to the linear RNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In one embodiment, a linear RNA comprises a lncRNA or a sequence of a lncRNA, e.g., a linear RNA comprises a sequence of a naturally occurring, non-circular lncRNA or a fragment thereof.

In one embodiment, a linear RNA has ribozyme activity. In one embodiment, a linear RNA can be used to act as a ribozyme and cleave pathogenic or endogenous RNA, DNA, small molecules, or proteins. In one embodiment, a linear RNA has enzymatic activity. In one embodiment linear RNA is able to specifically recognize and cleave proteins.

In one embodiment, a linear RNA is an immolating or self-cleaving or cleavable linear RNA.

In one embodiment, a linear RNA is a transcriptionally/replication competent linear RNA. This linear RNA can encode any type of RNA. In one embodiment, a synthetic linear RNA has an anti-sense miRNA and a transcriptional element. In one embodiment, after transcription, linear functional miRNAs are generated from a synthetic linear RNA.

In one embodiment, a linear RNA has one or more of the above attributes in combination with a translating element.

In some embodiments, a linear RNA comprises at least one modified nucleotide. In some embodiments, a linear RNA comprises 10%, 20%, 30%, 40%, 50%, 60%, 70%, or 80% modified nucleotides. In some embodiments, a linear RNA comprises substantially all (e.g., greater than 80%, 85%, 90%, 95%, 97%, 98%, or 99%, or about 100%) modified nucleotides. In some embodiments, a linear RNA comprises modified nucleotides and a portion of unmodified contiguous nucleotides, which can be referred to as a hybrid modified linear RNA. A portion of unmodified contiguous nucleotides can be an unmodified binding site configured to bind a protein, DNA, RNA, or a cell target in a hybrid modified linear RNA. A portion of unmodified contiguous nucleotides can be an unmodified IRES in a hybrid modified linear RNA. In other embodiments, a linear RNA lacks modified nucleotides, which is also referred to herein as an unmodified linear RNA.

In some embodiments, the linear RNA is an exogenous, synthetic linear RNA polyribonucleotide. In some embodiments, the linear RNA lacks a poly-A sequence, a replication element, or both. In some embodiments, the linear RNA polyribonucleotide as disclosed herein is translation incompetent.

### Binding Sites

In some embodiments, a linear RNA comprises one binding site. In some embodiments, a linear RNA comprises at least two binding sites. For example, a linear RNA can comprise 2 binding sites. For example, a linear RNA can comprise 3 binding sites. For example, a linear RNA can comprise 4 binding sites. For example, a linear RNA can comprise 5 binding sites. For example, a linear RNA can comprise 6 binding sites. For example, a linear RNA can comprise 7 binding sites. For example, a linear RNA can comprise 8 binding sites. For example, a linear RNA can comprise 9 binding sites. For example, a linear RNA can comprise 10 binding sites. For example, a linear RNA can comprise 11 binding sites. For example, a linear RNA can comprise 12 binding sites. For example, a linear RNA can comprise 13 binding sites. For example, a linear RNA can comprise 14 binding sites. For example, a linear RNA can comprise 15, 16, 17, 18, 19, 20, or more binding sites. For example, a linear RNA can comprise 16 binding sites. For example, a linear RNA can comprise 17 binding sites. For example, a linear RNA can comprise 18 binding sites. For example, a linear RNA can comprise 19, 20, or more binding sites. For example, a linear RNA can comprise 20 binding sites. In some embodiments, linear RNA described herein is a molecular scaffold that binds one or more targets. Each target may be, but is not limited to, a different or the same target protein. In some embodiments, linear RNA described herein is a molecular scaffold that binds one or more substrates of a target. Each substrate may be, but is not limited to, a different or the same as the target protein. In some embodiments, a linear RNA described herein is a molecular scaffold that binds one or more targets and one or more substrates of the target. In some embodiments, a linear RNA comprises an aptamer that bind one or more targets and one or substrates of the target. In some embodiments, a linear RNA comprises a conjugation moiety that is conjugated to a chemical compound, wherein the chemical compound binds to the target. In some embodiments, a linear RNA comprises a conjugation moiety that binds to a chemical compound, wherein the chemical compound binds to the substrate. In some embodiments, the target is a target protein. In some embodiments, the substrate is a substrate protein. In some embodiments, the linear RNA comprises a conjugation moiety that binds to a chemical compound and an aptamer that binds to a target (e.g., a target protein). In some embodiments, the linear RNA comprises a conjugation moiety that binds to a chemical compound and an aptamer that binds to a substrate (e.g., a substrate protein).

### Conjugation Moiety

A linear RNA can comprise a conjugation moiety. In some embodiments, a linear RNA comprises a conjugation moiety that is conjugated to a chemical compound, wherein the chemical compound binds to a target. In some embodiments, a linear RNA comprises a conjugation moiety that is conjugated to a chemical compound, wherein the chemical compound binds to a substrate. In some embodiments, a linear RNA comprises an aptamer that compound binds to a target. In some embodiments, a linear RNA comprises a conjugation moiety that is conjugated to a chemical compound, wherein the chemical compound binds to a substrate. A target can be a target protein. A substrate can be a substrate protein. The target protein can modulate the substrate protein. In some embodiments, the linear RNA comprises a conjugation moiety and an aptamer.

A conjugation moiety can be a modified nucleotide that facilitates conjugation to chemical compound. A conjugation moiety can be a modified nucleotide comprising a functional group that can be conjugated to a chemical compound. A conjugation moiety can be incorporated, for example, at a 5′ end of a linear RNA. A conjugation moiety can be incorporated, for example, at a 3′ end of a linear RNA. A conjuation moiety can be incorporated at an internal site of a linear RNA. A conjugation moiety can be a nucleotide analog, e.g., bromodeoxyuridine. A conjugation moiety can be a functional group, e.g., an azide group or an alkyne group. A conjugation moiety can be a hapten group, e.g., comprising digoxigenin, 2,4-dinitrophenyl, biotin, avidin, or are selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines.

A conjugation moiety can be conjugated via a chemical reaction, e.g., using click chemistry or a Staudinger reaction to chemical compound. The conjugation moiety can be a single modified nucleotide of choice (e.g., modified A, C, G, U, or T containing an azide at the 2′-position) that is incorporated site-specifically under optimized conditions (e.g., via solid-phase chemical synthesis). The conjugation moiety can be a plurality of nucleotides containing an azide at the 2′-position that are incorporated, for example, by substituting a nucleotide during an in vitro transcription reaction (e.g., substituting UTP for 5-azido-C3-UTP). Non-limiting examples of conjugation moeities include modified UTP analogs, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP, and 5-DBCO-PEG4-dCpG.

In some embodiments, a linear RNA comprises a plurality of conjugation moieties. For example, a linear RNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90 or 100 or more conjugation moieties or any number therein between. A linear RNA can comprise 1 conjugation moiety. A linear RNA can comprise 2 conjugation moieties. A linear RNA can comprise 3 conjugation moieties. A linear RNA can comprise 4 conjugation moieties. A linear RNA can comprise 5 conjugation moieties. A linear RNA can comprise 6 conjugation moieties. A linear RNA can comprise 7 conjugation moieties. A linear RNA can comprise 8 conjugation moieties. A linear RNA can comprise 9 conjugation moieties. A linear RNA can comprise 10 conjugation moieties. A linear RNA can comprise 11 conjugation moieties. A linear RNA can comprise 12 conjugation moieties. A linear RNA can comprise 13 conjugation moieties. A linear RNA can comprise 14 conjugation moieties. A linear RNA can comprise 15 conjugation moieties. A linear RNA can comprise 16 conjugation moieties. A linear RNA can comprise 17 conjugation moieties. A linear RNA can comprise 18 conjugation moieties. A linear RNA can comprise 19 conjugation moieties. A linear RNA can comprise 20 conjugation moieties. A linear RNA can comprise 21 conjugation moieties. A linear RNA can comprise 22 conjugation moieties. A linear RNA can comprise 23 conjugation moieties. A linear RNA can comprise 24 conjugation moieties. A linear RNA can comprise 25 conjugation moieties. A linear RNA can comprise 26 conjugation moieties. A linear RNA can comprise 27 conjugation moieties. A linear RNA can comprise 28 conjugation moieties. A linear RNA can comprise 29 conjugation moieties. A linear RNA can comprise 30 conjugation moieties. A linear RNA can comprise 31 conjugation moieties. A linear RNA can comprise 32 conjugation moieties. A linear RNA can comprise 33 conjugation moieties. A linear RNA can comprise 34 conjugation moieties. A linear RNA can comprise 35 conjugation moieties. A linear RNA can comprise 36 conjugation moieties. A linear RNA can comprise 37 conjugation moieties. A linear RNA can comprise 38 conjugation moieties. A linear RNA can comprise 39 conjugation moieties. A linear RNA can comprise 40 conjugation moieties. A linear RNA can comprise 41 conjugation moieties. A linear RNA can comprise 42 conjugation moieties. A linear RNA can comprise 43 conjugation moieties. A linear RNA can comprise 44 conjugation moieties. A linear RNA can comprise 45 conjugation moieties. A linear RNA can comprise 46 conjugation moieties. A linear RNA can comprise 47 conjugation moieties. A linear RNA can comprise 48 conjugation moieties. A linear RNA can comprise 49 conjugation moieties. A linear RNA can comprise 50 conjugation moieties. A linear RNA can comprise 55 conjugation moieties. A linear RNA can comprise 60 conjugation moieties. A linear RNA can comprise 70 conjugation moieties. A linear RNA can comprise 80 conjugation moieties. A linear RNA can comprise 90 conjugation moieties. A linear RNA can comprise 100 conjugation moieties. In some embodiments, the plurality of conjugation moieties are the same. In some embodiments, the plurality of conjugation moieties are different. In some embodiments, a linear RNA comprises a first conjugation moiety and a second conjugation moiety. In some embodiments, a linear RNA comprises a first conjugation moiety that is conjugated to a first chemical compound and a second conjugation moiety that is conjugated to a second chemical compound, wherein the first chemical compound binds to a target and the second chemical compound binds to a substrate of the target.

### Protein Binding Sites

In some embodiments, the linear polyribonucleotide comprises one or more binding sites that bind to a protein. The protein binding site can bind to a linear polyribonucleotide (linear RNA)-binding motif of a protein. The protein can be substrate protein. The protein can be target protein.

In some embodiments, a protein binding site comprises a chemical compound (e.g., a chemical compound conjugated to the linear RNA via a conjugation moiety). In some embodiments, a protein binding site comprises a protein binding sequence (e.g., an RNA sequence comprising a protein sequence-binding motif). In some embodiments, the protein binding sequence targets or localizes a linear polyribonucleotide to a specific substrate protein of a target protein. In some embodiments, the protein binding sequence specifically binds an arginine-rich region of a protein. In some embodiments, linear polyribonucleotides disclosed herein comprise a protein binding sequence that binds to protein substrate of an enzyme. In some embodiments, linear polyribonucleotides disclosed herein comprise a protein binding sequence that binds to disease-associated protein. In some embodiments, linear polyribonucleotides disclosed herein comprise a protein binding sequence that binds to protein associated with cancer. In some embodiments, linear polyribonucleotides disclosed herein comprise a protein binding sequence that binds to misfolded protein. In some embodiments, a protein binding site comprises a nucleic acid sequence that can bind to a protein such as BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, or MDM2.

In some instances, a protein binding site binds to portion of a protein comprising a span of at least 6 amino acids, for example, least 8, 9, 10, 12, 15, 20, 25, 30, 40, 50, or 100 amino acids. A protein binding site can bind to portion of a protein comprising a span of 6 amino acids. A protein binding site can bind to portion of a protein comprising a span of 8 amino acids. A protein binding site can bind to portion of a protein comprising a span of 9 amino acids. A protein binding site can bind to portion of a protein comprising a span of 10 amino acids. A protein binding site can bind to portion of a protein comprising a span of 12 amino acids. A protein binding site can bind to portion of a protein comprising a span of 15 amino acids. A protein binding site can bind to portion of a protein comprising a span of 20 amino acids. A protein binding site can bind to portion of a protein comprising a span of 25 amino acids. A protein binding site can bind to portion of a protein comprising a span of 30 amino acids. A protein binding site can bind to portion of a protein comprising a span of 40 amino acids. A protein binding site can bind to portion of a protein comprising a span of 50 amino acids. A protein binding site can bind to portion of a protein comprising a span of 100 amino acids. In some instances, a protein binding site binds to a portion of a protein comprising a contiguous stretch of amino acids. In some instances, a protein binding site binds to a portion of a protein comprising a non-contiguous stretch of amino acids. In some instances, a protein binding site binds to a portion of a protein comprising a site of a mutation or functional mutation, including a deletion, addition, swap, or truncation of the amino acids in a polypeptide sequence.

In some instances, a protein binding site of the linear polyribonucleotide binds to a polypeptide, protein, or fragment thereof. In some embodiments, a binding site binds to a domain, a fragment, an epitope, a region, or a portion of a polypeptide, protein, or fragment thereof. For example, a protein binding site binds to a domain, a fragment, an epitope, a region, or a portion of an isolated polypeptide, a polypeptide of a cell, a purified polypeptide, or a recombinant polypeptide. For example, a protein binding site binds to a domain, a fragment, an epitope, a region, or a portion of an antibody or fragment thereof. For example, a binding site binds to a domain, a fragment, an epitope, a region, or a portion of a transcription factor. For example, a protein binding site binds to a domain, a fragment, an epitope, a region, or a portion of a receptor. For example, a binding site binds to a domain, a fragment, an epitope, a region, or a portion of a transmembrane receptor. Protein binding sites may bind to a domain, a fragment, an epitope, a region, or a portion of isolated, purified, and/or recombinant polypeptides. Protein binding sites may bind to a domain, a fragment, an epitope, a region, or a portion of a mixture of analytes (e.g., a lysate). For example, a protein binding site binds to a domain, a fragment, an epitope, a region, or a portion of from a plurality of cells or from a lysate of a single cell.

In some embodiments, a protein binding site binds to a domain, a fragment, an epitope, a region, or a portion of a membrane bound protein. Exemplary membrane bound proteins include, but are not limited to, GPCRs (e.g. adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), receptor tyrosine kinases, receptor serine/threonine kinases, receptor guanylate cyclases, growth factor and hormone receptors (e.g., epidermal growth factor (EGF) receptor), and others. The binding site may bind to a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

A protein binding site can bind to a domain, a fragment, an epitope, a region, or a portion of a member of a specific binding pair (e.g., a ligand). A protein binding site can bind to a domain, a fragment, an epitope, a region, or a portion of monovalent (monoepitopic) or polyvalent (polyepitopic). A binding moiety can be antigenic or haptenic. A protein binding site can bind to a domain, a fragment, an epitope, a region, or a portion of a single molecule or a plurality of molecules that share at least one common epitope or determinant site. A protein binding site can bind to a domain, a fragment, an epitope, a region, or a portion of a part of a cell (e.g., a bacteria cell, a plant cell, or an animal cell).

In some instances, a protein binding site binds to a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated. Samples include a quantity of a substance from a living thing or formerly living things. A sample can be natural, recombinant, synthetic, or not naturally occurring. A binding site can bind to any of the above that is expressed from a cell naturally or recombinantly, in a cell lysate or cell culture medium, an in vitro translated sample, or an immunoprecipitation from a sample (e.g., a cell lysate).

In some instances, a protein binding site binds to a protein expressed in a cell-free system or in vitro. For example, a protein binding site binds to a protein in a cell extract. In some instances, a protein binding site binds to a protein in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and µIP-microintaglio printing, and pMAC-protein microarray copying (See Kilb et al., Eng. Life Sci. 2014, 14, 352-364).

In some instances, a protein binding site of a linear RNA is synthesized in situ (e.g., on a solid substrate of an array) from a DNA template. In some instances, a plurality of binding sites is synthesized in situ from a plurality of corresponding DNA templates in parallel or in a single reaction. Exemplary methods for in situ protein expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some embodiments, linear RNA further comprises other binding motifs for binding other intracellular molecules.

### RNA Binding Sites

In some embodiments, the linear polyribonucleotide further comprises one or more RNA binding sites. In some embodiments, the linear polyribonucleotide includes RNA binding sites that modify expression of an endogenous gene and/or an exogenous gene. In some embodiments, the RNA binding site modulates expression of a host gene. The RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor.

In some embodiments, the RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. RNA binding sites are well-known to persons of ordinary skill in the art.

The length of the RNA binding site may be between about 5 to 30 nucleotides. The length of the RNA binding site may be between about 10 to 30 nucleotides. The length of the RNA binding site may be about 11, 12, 13, 14, 15, 16, 17, 18, 19, 20, 21, 22, 23, 24, 25, 26, 27, 28, 29, 30, or more nucleotides. The length of the RNA binding site may be 11 nucleotides. The length of the RNA binding site may be 12 nucleotides. The length of the RNA binding site may be 13 nucleotides. The length of the RNA binding site may be 14 nucleotides. The length of the RNA binding site may be 15 nucleotides. The length of the RNA binding site may be 16 nucleotides. The length of the RNA binding site may be 17 nucleotides. The length of the RNA binding site may be 18 nucleotides. The length of the RNA binding site may be 19 nucleotides. The length of the RNA binding site may be 20 nucleotides. The length of the RNA binding site may be 21 nucleotides. The length of the RNA binding site may be 22 nucleotides. The length of the RNA binding site may be 23 nucleotides. The length of the RNA binding site may be 24 nucleotides. The length of the RNA binding site may be 25 nucleotides. The length of the RNA binding site may be 26 nucleotides. The length of the RNA binding site may be 27 nucleotides. The length of the RNA binding site may be 28 nucleotides. The length of the RNA binding site may be 29 nucleotides. The length of the RNA binding site may be 30 nucleotides. The degree of identity of the RNA binding site to a target of interest can be at least 75%. The degree of identity of the RNA binding site to a target of interest can be at least at least 80%, at least 85%. The degree of identity of the RNA binding site to a target of interest can be at least at least 90%. The degree of identity of the RNA binding site to a target of interest can be at least at least 95%.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

In some embodiments, the linear polyribonucleotide further comprises a RNA binding site that has an RNA or RNA-like structure typically between about 5-5000 base pairs (depending on the specific RNA structure, e.g., miRNA 5-30 bps, lncRNA 200-500 bps) and has a nucleobase sequence identical (complementary) or nearly identical (substantially complementary) to a coding sequence in an expressed target gene within the cell.

In some embodiments, a RNA binding site comprises a chemical compound (e.g., a chemical compound conjugated to the linear RNA via a conjugation moiety).

### DNA Binding Sites

In some embodiments, the linear polyribonucleotide further comprises a DNA binding site, such as a sequence for a guide RNA (gRNA). In some embodiments, the linear polyribonucleotide comprises a guide RNA or a complement to a gRNA sequence. A gRNA short synthetic RNA composed of a “scaffold” sequence necessary for binding to the incomplete effector moiety and a user-defined ~20 nucleotide targeting sequence for a genomic target. Guide RNA sequences can have a length of between 17 - 24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the linear polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage. The gRNA sequences may include 10 nucleotides for interaction with Cas9 or other exonuclease to cleave DNA. The gRNA sequences may include 11 nucleotides for interaction with Cas9 or other exonuclease to cleave DNA. The gRNA sequences may include 12 nucleotides for interaction with Cas9 or other exonuclease to cleave DNA. The gRNA sequences may include 13 nucleotides for interaction with Cas9 or other exonuclease to cleave DNA. The gRNA sequences may include 14 nucleotides for interaction with Cas9 or other exonuclease to cleave DNA. The gRNA sequences may include 15 nucleotides for interaction with Cas9 or other exonuclease to cleave DNA. The gRNA sequences may include 16 nucleotides for interaction with Cas9 or other exonuclease to cleave DNA. The gRNA sequences may include 17 nucleotides for interaction with Cas9 or other exonuclease to cleave DNA. The gRNA sequences may include 18 nucleotides for interaction with Cas9 or other exonuclease to cleave DNA. The gRNA sequences may include 19 nucleotides for interaction with Cas9 or other exonuclease to cleave DNA. The gRNA sequences may include 20 nucleotides for interaction with Cas9 or other exonuclease to cleave DNA. The gRNA sequences may include 21 nucleotides for interaction with Cas9 or other exonuclease to cleave DNA. The gRNA sequences may include 22 nucleotides for interaction with Cas9 or other exonuclease to cleave DNA. The gRNA sequences may include 23 nucleotides for interaction with Cas9 or other exonuclease to cleave DNA. The gRNA sequences may include 24 nucleotides for interaction with Cas9 or other exonuclease to cleave DNA. The gRNA sequences may include 25 nucleotides for interaction with Cas9 or other exonuclease to cleave DNA. The gRNA sequences may include 26 nucleotides for interaction with Cas9 or other exonuclease to cleave DNA. The gRNA sequences may include 27 nucleotides for interaction with Cas9 or other exonuclease to cleave DNA. The gRNA sequences may include 28 nucleotides for interaction with Cas9 or other exonuclease to cleave DNA. The gRNA sequences may include 29 nucleotides for interaction with Cas9 or other exonuclease to cleave DNA. The gRNA sequences may include 30 nucleotides for interaction with Cas9 or other exonuclease to cleave DNA.

In some embodiments, the linear polyribonucleotide includes sequences that bind a major groove of in duplex DNA. In one such instance, the specificity and stability of a triplex structure created by the linear polyribonucleotide and duplex DNA is afforded via Hoogsteen hydrogen bonds, which are different from those formed in classical Watson- Crick base pairing in duplex DNA. In one instance, the linear polyribonucleotide binds to the purine-rich strand of a target duplex through the major groove.

In some embodiments, triplex formation occurs in two motifs, distinguished by the orientation of the linear polyribonucleotide with respect to the purine-rich strand of the target duplex. In some instances, polypyrimidine sequence stretches in a linear polyribonucleotides bind to the polypurine sequence stretches of a duplex DNA via Hoogsteen hydrogen bonding in a parallel fashion (i.e. in the same 5′ to 3′, orientation as the purine-rich strand of the duplex), whereas the polypurine stretches (R) bind in an antiparallel fashion to the purine strand of the duplex via reverse-Hoogsteen hydrogen bonds. In the antiparallel, a purine motif comprises triplets of G:G-C, A:A-T, or T:A-T; whereas in the parallel, a pyrimidine motif comprises canonical triples of C+:G-C or T:A-T triplets (where C+ represents a protonated cytosine on the N3 position). Antiparallel GA and GT sequences in a linear polyribonucleotide may form stable triplexes at neutral pH, while parallel CT sequences in a linear polyribonucleotide may bind at acidic pH. N3 on cytosine in the linear polyribonucleotide may be protonated. Substitution of C with 5-methyl-C may permit binding of CT sequences in the linear polyribonucleotide at physiological pH as 5-methyl-C has a higher pK than does cytosine. For both purine and pyrimidine motifs, contiguous homopurine-homopyrimidine sequence stretches of at least 10 base pairs aid linear polyribonucleotide binding to duplex DNA, since shorter triplexes may be unstable under physiological conditions, and interruptions in sequences can destabilize the triplex structure. In some embodiments, the DNA duplex target for triplex formation includes consecutive purine bases in one strand. In some embodiments, a target for triplex formation comprises a homopurine sequence in one strand of the DNA duplex and a homopyrimidine sequence in the complementary strand.

In some embodiments, a triplex comprising a linear polyribonucleotide is a stable structure. In some embodiments, a triplex comprising a linear polyribonucleotide exhibits an increased half-life, e.g., increased by about 5%, 10%, 15%, 20%, 25%, 30%, 35%, 40%, 45%, 50%, or greater, e.g., persistence for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In some embodiments, a triplex comprising a linear polyribonucleotide exhibits a half-life increased by 5%. In some embodiments, a triplex comprising a linear polyribonucleotide exhibits a half-life increased by 10%. In some embodiments, a triplex comprising a linear polyribonucleotide exhibits a half-life increased by 15%. In some embodiments, a triplex comprising a linear polyribonucleotide exhibits a half-life increased by 20%. In some embodiments, a triplex comprising a linear polyribonucleotide exhibits a half-life increased by 25%. In some embodiments, a triplex comprising a linear polyribonucleotide exhibits a half-life increased by 30%. In some embodiments, a triplex comprising a linear polyribonucleotide exhibits a half-life increased by 35%. In some embodiments, a triplex comprising a linear polyribonucleotide exhibits a half-life increased by 40%. In some embodiments, a triplex comprising a linear polyribonucleotide exhibits a half-life increased by 45%. In some embodiments, a triplex comprising a linear polyribonucleotide exhibits a half-life increased by 50%. In some embodiments, a triplex comprising a linear polyribonucleotide exhibits persistence for 1 hr to about 30 days. In some embodiments, a triplex comprising a linear polyribonucleotide exhibits persistence for 2 hrs. In some embodiments, a triplex comprising a linear polyribonucleotide exhibits persistence for 6 hrs. In some embodiments, a triplex comprising a linear polyribonucleotide exhibits persistence for 12 hrs. In some embodiments, a triplex comprising a linear polyribonucleotide exhibits persistence for 18 hrs. In some embodiments, a triplex comprising a linear polyribonucleotide exhibits persistence for 24 hrs. In some embodiments, a triplex comprising a linear polyribonucleotide exhibits persistence for 2 days. In some embodiments, a triplex comprising a linear polyribonucleotide exhibits persistence for 3 days. In some embodiments, a triplex comprising a linear polyribonucleotide exhibits persistence for 4 days. In some embodiments, a triplex comprising a linear polyribonucleotide exhibits persistence for 5 days. In some embodiments, a triplex comprising a linear polyribonucleotide exhibits persistence for 6 days. In some embodiments, a triplex comprising a linear polyribonucleotide exhibits persistence for 7 days. In some embodiments, a triplex comprising a linear polyribonucleotide exhibits persistence for 8 days. In some embodiments, a triplex comprising a linear polyribonucleotide exhibits persistence for 9 days. In some embodiments, a triplex comprising a linear polyribonucleotide exhibits persistence for 10 days. In some embodiments, a triplex comprising a linear polyribonucleotide exhibits persistence for 11 days. In some embodiments, a triplex comprising a linear polyribonucleotide exhibits persistence for 12 days. In some embodiments, a triplex comprising a linear polyribonucleotide exhibits persistence for 13 days. In some embodiments, a triplex comprising a linear polyribonucleotide exhibits persistence for 14 days. In some embodiments, a triplex comprising a linear polyribonucleotide exhibits persistence for 15 days. In some embodiments, a triplex comprising a linear polyribonucleotide exhibits persistence for 16 days. In some embodiments, a triplex comprising a linear polyribonucleotide exhibits persistence for 17 days. In some embodiments, a triplex comprising a linear polyribonucleotide exhibits persistence for 18 days. In some embodiments, a triplex comprising a linear polyribonucleotide exhibits persistence for 19 days. In some embodiments, a triplex comprising a linear polyribonucleotide exhibits persistence for 20 days. In some embodiments, a triplex comprising a linear polyribonucleotide exhibits persistence for 21 days. In some embodiments, a triplex comprising a linear polyribonucleotide exhibits persistence for 22 days. In some embodiments, a triplex comprising a linear polyribonucleotide exhibits persistence for 23 days. In some embodiments, a triplex comprising a linear polyribonucleotide exhibits persistence for 24 days. In some embodiments, a triplex comprising a linear polyribonucleotide exhibits persistence for 25 days. In some embodiments, a triplex comprising a linear polyribonucleotide exhibits persistence for 26 days. In some embodiments, a triplex comprising a linear polyribonucleotide exhibits persistence for 27 days. In some embodiments, a triplex comprising a linear polyribonucleotide exhibits persistence for 28 days. In some embodiments, a triplex comprising a linear polyribonucleotide exhibits persistence for 29 days. In some embodiments, a triplex comprising a linear polyribonucleotide exhibits persistence for 30 days. In some embodiments, a triplex comprising a linear polyribonucleotide exhibits persistence for 60 days.

In some embodiments, a DNA binding site comprises a chemical compound (e.g., a chemical compound conjugated to the linear RNA via a conjugation moiety).

### Other Binding Sites

In some embodiments, the linear RNA further comprises a binding site that binds to one of a small molecule, an aptamer, a lipid, a carbohydrate, a virus particle, a membrane, a multi-component complex, a cell, a cellular moiety, or any fragment thereof binding site. In some embodiments, the linear polyribonucleotide further comprises one or more binding sites that bind to a lipid. In some embodiments, the linear polyribonucleotide comprises one or more binding sites that bind to a carbohydrate. In some embodiments, the linear polyribonucleotide further comprises one or more binding sites that bind to a carbohydrate. In some embodiments, the linear polyribonucleotide further comprises one or more binding sites that bind to a membrane. In some embodiments, the linear polyribonucleotide further comprises one or more binding sites that bind to a multi-component complex, e.g., ribosome, nucleosome, transcription machinery, etc. In some embodiments, a binding site comprises a chemical compound (e.g., a chemical compound conjugated to the linear RNA via a conjugation moiety).

### Modifications

In some aspects, the invention described herein comprises compositions and methods of using and making modified linear polyribonucleotides, and delivery of modified linear polyribonucleotides. The term “modified nucleotide” can refer to any nucleotide analog or derivative that has one or more chemical modifications to the chemical composition of an unmodified natural ribonucleotide, such as a natural unmodified nucleotide adenosine (A), uridine (U), guaninie (G), cytidine (C) as shown by the chemical formulae in TABLE 6, and monophosphate. The chemical modifications of the modified ribonucleotide can be modifications to any one or more functional groups of the ribonucleotide, such as, the sugar the nucleobase, or the internucleoside linkage (e.g. to a linking phosphate / to a phosphodiester linkage / to the phosphodiester backbone).

TABLE 1

UNMODIFIED NATURAL RIBONUCLEOSIDES

| | | |
|---|---|---|
| Ribonucleoside | IUPAC name | Chemical Formula |
| Adenosine | (2R,3R,4S,5R)-2-(6-amino-9H-purin-9-yl)-5-(hydroxymethyl)oxolane-3,4-diol | 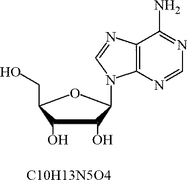 |
| Uridine | 1-[(3R,4S,5R)-3,4-dihydroxy-5-(hydroxymethyl)oxolan-2-yl]pyrimidine-2,4-dione | 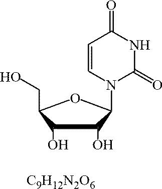 |
| Guanine | 2-amino-9H-purin-6(1H)-one | 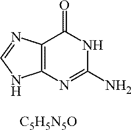 [SMILES: Nc1nc2[nH]cnc2c(=O)[nH]1 ; "C5H5N5O"] |
| Cytidine | 4-amino-1-[(2R,3R,4S,5R)-3,4-dihydroxy-5-(hydroxymethyl)oxolan-2-yl]pyrimidin-2(1H)-one | 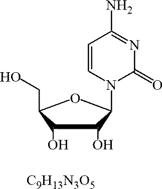 |

The linear polyribonucleotide can include one or more substitutions, insertions and/or additions, deletions, and covalent modifications with respect to reference sequences, in particular, the parent polyribonucleotide, are included within the scope of this invention. In some embodiments, the linear polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The linear polyribonucleotide can include any useful modification, such as to the sugar, the nucleobase, or the internucleoside linkage (e.g., to a linking phosphate / to a phosphodiester linkage / to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications can be modifications of ribonucleic acids (RNA) to deoxyribonucleic acids (DNA), threose nucleic acids (TNA), glycol nucleic acids (GNA), peptide nucleic acids (PNA), locked nucleic acids (LNA) or hybrids thereof). Additional modifications are described herein.

In some embodiments, the linear polyribonucleotide includes at least one N(6)methyladenosine (m6A) modification to increase translation efficiency.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

“Pseudouridine” refers, in another embodiment, to m¹acp³Ψ (1-methyl-3-(3-amino-3-carboxypropyl) pseudouridine. In another embodiment, the term refers to m¹Ψ (1-methylpseudouridine). In another embodiment, the term refers to Ψm (2′-O-methylpseudouridine. In another embodiment, the term refers to m5D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

In some embodiments, chemical modifications to the ribonucleotides of the linear polyribonucleotide can enhance immune evasion. Modifications include, for example, end modifications, e.g., 5′-end modifications (phosphorylation (mono-, di- and tri-), conjugation, inverted linkages, etc.), 3′-end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases can also include 5-methylcytidine and pseudouridine. In some embodiments, base modifications can modulate expression, immune response, stability, subcellular localization, to name a few functional effects, of the linear polyribonucleotide. In some embodiments, the modification includes a bi-orthogonal nucleotide, e.g., an unnatural base.

In some embodiments, sugar modifications (e.g., at the 2′ position or 4′ position) or replacement of the sugar one or more ribonucleotides of the linear polyribonucleotide can, as well as backbone modifications, include modification or replacement of the phosphodiester linkages. Non-limiting examples of linear polyribonucleotide include linear polyribonucleotide with modified backbones or non-natural internucleoside linkages, such as those modified or replaced of the phosphodiester linkages. Linear polyribonucleotides having modified backbones include, among others, those that do not have a phosphorus atom in the backbone. For the purposes of this application, and as sometimes referenced in the art, modified RNA that do not have a phosphorus atom in their internucleoside backbone can also be considered to be oligonucleosides. In particular embodiments, the linear polyribonucleotide will include ribonucleotides with a phosphorus atom in its internucleoside backbone.

Modified linear polyribonucleotide backbones can include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the linear polyribonucleotide can be negatively or positively charged.

The modified nucleotides, which can be incorporated into the linear polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene -phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the linear polyribonucleotide is expected to reduce the innate immune response through weaker binding/activation of cellular innate immune molecules.

In some embodiments, a modified nucleoside includes an α-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine). Other internucleoside linkages can include internucleoside linkages which do not contain a phosphorous atom.

In some embodiments, the linear polyribonucleotide can include one or more cytotoxic nucleosides. For example, cytotoxic nucleosides can be incorporated into linear polyribonucleotide, such as bifunctional modification. Cytotoxic nucleoside can include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((R,S)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-l-(2-C-cyano-2-deoxy-beta-D-arabino-pentofuranosyl) cytosine, and P-4055 (cytarabine 5′-elaidic acid ester).

The linear polyribonucleotide can be uniformly modified along the entire length of the molecule. For example, one or more or all types of nucleotide (e.g., naturally-occurring nucleotides, purine or pyrimidine, or any one or more or all of A, G, U, C, I, pU) can be uniformly modified in the linear polyribonucleotide, or in a given predetermined sequence region thereof. In some embodiments, the linear polyribonucleotide includes a pseudouridine. In some embodiments, the linear polyribonucleotide includes an inosine, which can aid in the immune system characterizing the linear polyribonucleotide as endogenous versus viral RNA. The incorporation of inosine can also mediate improved RNA stability/reduced degradation.

In some embodiments, all nucleotides in the linear polyribonucleotide (or in a given sequence region thereof) are modified. In some embodiments, the modification can include an m6A, which can augment expression; an inosine, which can attenuate an immune response; pseudouridine, which can increase RNA stability, or translational readthrough (stop codon = coding potential), an m5C, which can increase stability; and a 2,2,7-trimethylguanosine, which aids subcellular translocation (e.g., nuclear localization).

Different sugar modifications, nucleotide modifications, and/or internucleoside linkages (e.g., backbone structures) can exist at various positions in the linear polyribonucleotide. One of ordinary skill in the art will appreciate that the nucleotide analogs or other modification(s) can be located at any position(s) of the linear polyribonucleotide, such that the function of the linear polyribonucleotide is not substantially decreased. A modification can also be a non-coding region modification. The linear polyribonucleotide can include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the linear polyribonucleotide provided herein is a modified linear polyribonucleotide. For example, a completely modified linear polyribonucleotide comprises all or substantially all modified adenosine residues, all or substantially all modified uridine residues, all or substantially all modified guanine residues, all or substantially all modified cytidine residues, or any combination thereof. In some embodiments, the linear polyribonucleotide provided herein is a hybrid modified linear polyribonucleotide. A hybrid modified linear polyribonucleotide can have at least one modified nucleotide and can have a portion of contiguous unmodified nucleotides. This unmodified portion of the hybrid modified linear polyribonucleotide can have at least about 5, 10, 15, or 20 contiguous unmodified nucleotides, or any number therebetween. In some embodiments, the unmodified portion of the hybrid modified linear polyribonucleotide has at least about 30, 40, 40, 60, 70, 80, 90, 100, 110, 120, 130, 140, 150, 160, 180, 200, 220, 250, 280, 300, 320, 350, 380, 400, 420, 450, 500, 550, 600, 650, 700, 750, 800, 850, 900, or 1000 contiguous unmodified nucleotides, or any number therebetween. In some embodiments, the hybrid modified linear polyribonucleotide has 1, 2, 3, 4, 5, 6, 7, 8, 9, 10, or more unmodified portions. In some embodiments, the hybrid modified linear polyribonucleotide has at least 1, 2, 3, 4, 5, 6, 7, 8, 9, 10, 12, 15, 20, 30, 40, 50, 70, 80, 100, 120, 150, 200, 250, 300, 400, 500, 600, 700, 800, 900, 1000, or more modified nucleotides. In some embodiments, the hybrid modified linear polyribonucleotide has at least 1%, 2%, 5%, 7%, 8%, 10%, 12%, 15%, 18%, 20%, 25%, 30%, 35%, 40%, 45%, 50%, 55%, 60%, 65%, 70%, 80%, 90%, 95%, or 99% but less than 100% nucleotides that are modified. In some embodiments, the unmodified portion comprises a binding site. In some embodiments, the unmodified portion comprises a binding site configured to bind a protein, DNA, RNA, or a cell target.

In some embodiments, the hybrid modified linear polyribonucleotide has a lower immunogenicity than a corresponding unmodified linear polyribonucleotide. In some embodiments, the hybrid modified linear polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified linear polyribonucleotide. In some embodiments, the immunogenicity as described herein is assessed by the level of expression or signaling or activation of at least one of RIG-I, TLR-3, TLR-7, TLR-8, MDA-5, LGP-2, OAS, OASL, PKR, and IFN-beta. In some embodiments, the hybrid modified linear polyribonucleotide has a higher half-life than a corresponding unmodified linear polyribonucleotide. In some embodiments, the hybrid modified linear polyribonucleotide has a half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified linear polyribonucleotide. In some embodiments, the half-life is measured by introducing the linear polyribonucleotide or the corresponding unmodified linear polyribonucleotide into a cell and measuring a level of the introduced linear polyribonucleotide or corresponding unmodified linear polyribonucleotide inside the cell.

In some embodiments, the hybrid modified linear polyribonucleotide has a binding site that is unmodified, e.g., having no modified nucleotides. In some embodiments, the hybrid modified linear polyribonucleotide has a binding site configured to bind to a protein, DNA, RNA, or cell target that is unmodified, e.g., having no modified nucleotides. In some embodiments, the hybrid modified linear polyribonucleotide has no more than 10% of the nucleotides in the binding site that are modified nucleotides. In some embodiments, the hybrid modified linear polyribonucleotide has no more than 10% of the nucleotides in the binding site configured to bind to a protein, DNA, RNA, or cell target that are modified nucleotides. In some embodiments, a hybrid modified linear polyribonucleotide has modified nucleotides throughout except the binding site. In some embodiments, a hybrid modified linear polyribonucleotide has modified nucleotides throughout except the binding site configured to bind a protein, DNA, RNA, or a cell target.

In some embodiments, the linear polyribonucleotide is fully modified and has modified nucleotides throughout the linear polyribonucleotide. A fully modified linear polyribonucleotide can have increased stability and/or half-life. In some embodiments, the hybrid modified linear polyribonucleotide has modified nucleotides, e.g., 5′ methylcytidine and pseudouridine, throughout the linear polyribonucleotide. A hybrid modified linear polyribonucleotide can have improved binding of proteins compared to an unmodified linear polyribonucleotide. In these cases, the fully modified linear polyribonucleotide or hybrid modified linear polyribonucleotide has a higher a lower immnogeneicity as compared to a corresponding linear polyribonucleotide that is not fully modified or does not comprise 5′ methylcytidine and pseudouridine, respectively. In some embodiments, the fully modified linear polyribonucleotide or hybrid modified linear polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified linear polyribonucleotide. In some embodiments, the immunogenicity as described herein is assessed by expression or signaling or activation of at least one of RIG-I, TLR-3, TLR-7, TLR-8, MDA-5, LGP-2, OAS, OASL, PKR, and IFN-beta. In some embodiments, the fully modified linear polyribonucleotide or hybrid modified linear polyribonucleotide has a higher half-life than a corresponding unmodified linear polyribonucleotide, e.g., a corresponding linear polyribonucleotide that is not fully modified or does not comprise 5′ methylcytidine and pseudouridine, respectively. In some embodiments, the fully modified linear polyribonucleotide or hybrid modified linear polyribonucleotide has a higher half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified linear polyribonucleotide. In some embodiments, the half-life is measured by introducing the linear polyribonucleotide or the corresponding linear polyribonucleotide into a cell and measuring a level of the introduced linear polyribonucleotide or corresponding linear polyribonucleotide inside the cell.

In some cases, the fully modified linear polyribonucleotide or hybrid modified linear polyribonucleotide as described herein has similar immunogenicity as compared to a corresponding linear polyribonucleotide that is otherwise the same but completely modified.

### Other Linear Polyribonucleotide Features

In some embodiments, the linear polyribonucleotide comprises one or more of the elements as described herein in addition to comprising at a binding site and/or conjugation moiety. In some embodiments, the linear polyribonucleotide lacks a replication element. In some embodiments, the linear polyribonucleotide lacks an IRES. In some embodiments, the linear polyribonucleotide comprises any feature or any combination of features as disclosed below.

### 5′-Cap

In some embodiments, the linear polyribonucleotides include a 5′ cap, wherein the 5′ cap structure increases linear polyribonucleotide stability. The 5′ cap binds to the mTNA cap Binding Protein (MBP), which is responsible for increased stability of the linear polyribonucleotide in the cell and translation competency through the association of CBP with the poly-A binding protein.

In some embodiments, the endogenous linear polyribonucletioide molecules are 5′ end capped generating a 5′-ppp-5′triphosphate linkage between a terminal guanosine cap residue and the 5′ terminal transcribed sense nucleotide of the endogenous transcribed polyribonucleotide. This 5′ guanosine cap, also known as a 5′ guanlyated cap, can be methylated to generate a N7-methyl-guanylate cap.

### Encryptogen

As described herein, a linear polyribonucleotide can comprise an encryptogen to reduce, evade, or avoid the innate immune response of a cell. In some embodiments, linear polyribonucleotides provided herein result in a reduced immune response from the host as compared to the response triggered by a reference compound, e.g., a linear polynucleotide corresponding to the described linear polyribonucleotide or a linear polyribonucleotide lacking an encryptogen. In some embodiments, the linear polyribonucleotide has less immunogenicity than a counterpart lacking an encryptogen.

In some embodiments, the linear polyribonucleotide is non-immunogenic in a mammal, e.g., a human. In some embodiments, the linear polyribonucleotide is capable of replicating in a mammalian cell, e.g., a human cell.

In some embodiments, the linear polyribonucleotide includes sequences or expression products.

In some embodiments, the linear polyribonucleotide has a half-life of at least that of a linear counterpart, e.g., linear expression sequence, or linear linear polyribonucleotide. In some embodiments, the linear polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the linear polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the linear polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the linear polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the linear polyribonucleotide is at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs. In some embodiments, the linear polyribonucleotide can be of a sufficient size to accommodate a binding site for a ribosome. One of skill in the art can appreciate that the maximum size of a linear polyribonucleotide can be as large as is within the technical constraints of producing a linear polyribonucleotide, and/or using the linear polyribonucleotide. While not being bound by theory, it is possible that multiple segments of RNA can be produced from DNA and their 5′ and 3′ free ends annealed to produce a “string” of RNA. In some embodiments, the maximum size of a linear polyribonucleotide can be limited by the ability of packaging and delivering the RNA to a target. In some embodiments, the size of a linear polyribonucleotide is a length sufficient to encode useful polypeptides, and thus, lengths of less than about 20,000 base pairs, less than about 15,000 base pairs, less than about 10,000 base pairs, less than about 7,500 base pairs, or less than about 5,000 base pairs, less than about 4,000 base pairs, less than about 3,000 base pairs, less than about 2,000 base pairs, less than about 1,000 base pairs, less than about 500 base pairs, less than about 400 base pairs, less than about 300 base pairs, less than about 200 base pairs, less than about 100 base pairs can be useful.

### Translating Element

In some embodiments, any of the methods of using linear RNA described herein can be in combination with a translating element. Linear RNA described herein that contain a translating element can translate RNA into proteins. Protein expression can be facilitated by a linear RNA containing a sequence-specific RNA-binding motif, sequence-specific DNA-binding motif, protein-specific binding motif, and regulatory RNA motif. The regulatory RNA motif can initiate RNA transcription and protein expression.

### Untranslated Regions

In some embodiments, a linear RNA as disclosed herein can comprise an untranslated region (UTR). UTRs of a gene can be transcribed but not translated. In some embodiments, a UTR can be included upstream of the translation initiation sequence of an expression sequence described herein. In some embodiments, a UTR can be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

In some embodiments, the UTR enhances stability. In some embodiments, the regulatory features of a UTR can be included in the encryptogen to enhance the stability of the linear polyribonucleotide.

In some embodiments, the linear polyribonucleotide comprises a UTR with one or more stretches of adenosines and uridines embedded within. AU-rich signatures can increase turnover rates of the expression product.

Introduction, removal, or modification of UTR AU-rich elements (AREs) can be useful to modulate the stability or immunogenicity of the linear polyribonucleotide. When engineering specific linear polyribonucleotides, one or more copies of an ARE can be introduced to destabilize the linear polyribonucleotide and the copies of an ARE can decrease translation and/or decrease production of an expression product. Likewise, AREs can be identified and removed or mutated to increase the intracellular stability and thus increase translation and production of the resultant protein.

A UTR from any gene can be incorporated into the respective flanking regions of the linear polyribonucleotide (e.g., at the 5′ end or the 3′ end). Furthermore, multiple wild-type UTRs of any known gene can be utilized. In some embodiments, artificial UTRs that are not variants of wild type genes can be used. These UTRs or portions thereof can be placed in the same orientation as in the transcript from which they were selected or can be altered in orientation or location. Hence a 5′- or 3′-UTR can be inverted, shortened, lengthened, or made chimeric with one or more other 5′- or 3′-UTRs. As used herein, the term “altered” as it relates to a UTR sequence, means that the UTR has been changed in some way in relation to a reference sequence. For example, a 3′ - or 5′-UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double UTR, triple UTR, or quadruple UTR, such as a 5′- or 3′-UTR, can be used. As used herein, a “double” UTR is one in which two copies of the same UTR are encoded either in series or substantially in series. For example, a double beta-globin 3′-UTR can be used in some embodiments of the invention

### Cleavage Sequences

In some embodiments, the linear polyribonucleotide includes at least one cleavage sequence. In some embodiments, the cleavage sequence is adjacent to an expression sequence. In some embodiments, the linear polyribonucleotide includes a cleavage sequence, such as in an immolating linear RNA or cleavable linear RNA or self-cleaving linear RNA. In some embodiments, the linear polyribonucleotide comprises two or more cleavage sequences, leading to separation of the linear polyribonucleotide into multiple products, e.g., miRNAs, smaller linear polyribonucleotide, etc.

In some embodiments, the cleavage sequence includes a ribozyme RNA sequence. A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

### Immolating Sequence

In some embodiments, linear RNA described herein comprises immolating linear RNA or cleavable linear RNA or self-cleaving linear RNA. Linear RNA can deliver cellular components including, for example, RNA, lncRNA, lincRNA, miRNA, tRNA, rRNA, snoRNA, ncRNA, siRNA, or shRNA. In some embodiments, linear RNA includes miRNA separated by (i) self-cleavable elements; (ii) cleavage recruitment sites; (iii) degradable linkers; (iv) chemical linkers; and/or (v) spacer sequences. In some embodiments, linear RNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes. Non-limiting examples of linear RNA immolating applications are listed in TABLE 2.

TABLE 2

| | |
|---|---|
| PROCESS | MOA (example) |
| miRNA delivery | microRNAs in a linear form with self cleavage element (e.g., hammerhead), cleavage recruitment (e.g., ADAR) or degradable linker (glycerol) |
| siRNA delivery | siRNAs in a linear form with self cleavage element (e.g., hammerhead), cleavage recruitment (e.g., ADAR) or degradable linker (glycerol) |

### End-Protectants

In some embodiments, the linear polyribonucleotide comprises end protectants to improve resistance to degradation. In some embodiments, the protectants are G-quadruplexes, pseudoknots, stable terminal stem loops, poly-A tail, U-rich expression, or nuclear retention elements (ENE). In some embodiments, the end protectants can be on the 5′ and/or 3′ end of the polyribonucleotide. In some embodiments, the 5′ and/or 3′ end comprises a conjugation moiety. In some embodiments, the protectants are end modifications such as modifications on the N terminus ribonucleic acid or C terminus ribonucleic acid of the linear polyribonucleotide, modifications on the phosphodiester linkage, modifications on the sugar ring and modifications on the bases, the 3′ end capping with inverted thymidine, and PEGylation.

### Expression Sequences

### Peptides or Polypeptides

In some embodiments, the linear polyribonucleotide comprises a sequence that encodes a peptide or polypeptide.

The polypeptide can be linear or branched. The polypeptide can have a length from about 5 to about 4000 amino acids, about 15 to about 3500 amino acids, about 20 to about 3000 amino acids, about 25 to about 2500 amino acids, about 50 to about 2000 amino acids, or any range there between. In some embodiments, the polypeptide has a length of less than about 4000 amino acids, less than about 3500 amino acids, less than about 3000 amino acids, less than about 2500 amino acids, or less than about 2000 amino acids, less than about 1500 amino acids, less than about 1000 amino acids, less than about 900 amino acids, less than about 800 amino acids, less than about 700 amino acids, less than about 600 amino acids, less than about 500 amino acids, less than about 400 amino acids, less than about 300 amino acids, or less can be useful.

In some embodiments, the linear polyribonucleotide comprises one or more RNA sequences, each of which can encode a polypeptide. The polypeptide can be produced in substantial amounts. As such, the polypeptide can be any proteinaceous molecule that can be produced. A polypeptide can be a polypeptide that can be secreted from a cell, or localized to the cytoplasm, nucleus or membrane compartment of a cell.

In some embodiments, the linear polyribonucleotide includes a sequence encoding a protein e.g., a therapeutic protein. Some examples of therapeutic proteins can include, but are not limited to, an protein replacement, protein supplementation, vaccination, antigens (e.g., tumor antigens, viral, and bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

### Regulatory Sequences

In some embodiments, the regulatory sequence is a promoter. In some embodiments, the linear polyribonucleotide includes at least one promoter adjacent to at least one expression sequence. In some embodiments, the linear polyribonucleotide includes a promoter adjacent each expression sequence. In some embodiments, the promoter is present on one or both sides of each expression sequence, leading to separation of the expression products, e.g., peptide(s) and or polypeptide(s).

The linear polyribonucleotide can modulate expression of RNA encoded by a gene. Because multiple genes can share some degree of sequence homology with each other, the linear polyribonucleotide can be designed to target a class of genes with sufficient sequence homology. In some embodiments, the linear polyribonucleotide can contain a sequence that has complementarity to sequences that are shared amongst different gene targets or are unique for a specific gene target. In some embodiments, the linear polyribonucleotide can be designed to target conserved regions of an RNA sequence having homology between several genes thereby targeting several genes in a gene family. In some embodiments, the linear polyribonucleotide can be designed to target a sequence that is unique to a specific RNA sequence of a single gene.

In some embodiments, the expression sequence has a length less than 5000bps (e.g., less than about 5000bps, 4000bps, 3000bps, 2000bps, 1000bps, 900bps, 800bps, 700bps, 600bps, 500bps, 400bps, 300bps, 200bps, 100bps, 50bps, 40bps, 30bps, 20bps, 10bps, or less). In some embodiments, the expression sequence has, independently or in addition to, a length greater than 10bps (e.g., at least about 10bps, 20bps, 30bps, 40bps, 50bps, 60bps, 70bps, 80bps, 90bps, 100bps, 200bps, 300bps, 400bps, 500bps, 600bps, 700bps, 800bps, 900bps, 1000kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, the expression sequence comprises one or more of the features described herein, e.g., a sequence encoding one or more peptides or proteins, one or more regulatory nucleic acids, one or more non-coding RNA, and other expression sequences.

### Translation Initiation Sequence

In some embodiments, the linear polyribonucleotide encodes a polypeptide and can comprise a translation initiation sequence, e.g., a start codon. In some embodiments, the translation initiation sequence includes a Kozak or Shine-Dalgarno sequence. In some embodiments, the linear polyribonucleotide includes the translation initiation sequence, e.g., Kozak sequence, adjacent to an expression sequence. In some embodiments, the translation initiation sequence, e.g., Kozak sequence, is present on one or both sides of each expression sequence, leading to separation of the expression products. In some embodiments, the linear polyribonucleotide includes at least one translation initiation sequence adjacent to an expression sequence.

Natural 5′-UTRs can bear features that play a role in translation initiation. Natural 5′-UTRs can harbor signatures like Kozak sequences, which can be involved in the process by which the ribosome initiates translation of many genes. Kozak sequences have the consensus CCR(A/G)CCAUGG, where R is a purine (adenine or guanine) three bases upstream of the start codon (AUG), which is followed by another “G”. 5′-UTR can also form secondary structures that are involved in elongation factor binding.

The linear polyribonucleotide can include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60, or more than 60 start codons. Translation can initiate on the first start codon or initiate downstream of the first start codon.

In some embodiments, the linear polyribonucleotide can initiate at a codon that is not the first start codon, e.g., AUG. Translation of the linear polyribonucleotide can initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the linear polyribonucleotide can begin at alternative translation initiation sequence, such as ACG. As another non-limiting example, the linear polyribonucleotide translation can begin at alternative translation initiation sequence, CTG/CUG. As yet another non-limiting example, the linear polyribonucleotide translation can begin at alternative translation initiation sequence, GTG/GUG. As yet another non-limiting example, the linear polyribonucleotide can begin translation at a repeat-associated non-AUG (RAN) sequence, such as an alternative translation initiation sequence that includes short stretches of repetitive RNA, e.g., CGG, GGGGCC, CAG, CTG.

Nucleotides flanking a codon that initiates translation can affect the translation efficiency, the length and/or the structure of the linear polyribonucleotide. Masking any of the nucleotides flanking a codon that initiates translation can be used to alter the position of translation initiation, translation efficiency, length, and/or structure of the linear polyribonucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon-junction complexes (EJCs). In some embodiments, a masking agent can be used to mask a start codon of the linear polyribonucleotide in order to increase the likelihood that translation will initiate at an alternative start codon.

In some embodiments, translation is initiated under selective conditions, such as, but not limited to, viral induced selection in the presence of GRSF-1 and the linear polyribonucleotide includes GRSF-1 binding sites.

In some embodiments, translation is initiated by eukaryotic initiation factor 4A (eIF4A) treatment with Rocaglates. Translation can be repressed by blocking 43S scanning, leading to premature, upstream translation initiation and reduced protein expression from transcripts bearing the RocA-eIF4A target sequence.

### Termination Sequence

In some embodiments, the linear polyribonucleotide includes one or more expression sequences and each expression sequence can have a termination sequence. In some embodiments, the linear polyribonucleotide includes one or more expression sequences and the expression sequences lack a termination sequence, such that the linear polyribonucleotide is continuously translated. Exclusion of a termination sequence can result in rolling circle translation or continuous production of expression product, e.g., peptides or polypeptides, due to lack of ribosome stalling or fall-off. In such an embodiment, rolling circle translation produces a continuous expression product through each expression sequence.

In some embodiments, the linear polyribonucleotide includes a stagger sequence. To avoid production of a continuous expression product, e.g., peptide or polypeptide, while maintaining rolling circle translation, a stagger sequence can be included to induce ribosomal pausing during translation. The stagger sequence can include a 2A-like or CHYSEL (cis-acting hydrolase element) sequence. In some embodiments, the stagger element encodes a sequence with a C-terminal consensus sequence that is X1X2X3EX5NPGP, where X1 is absent or G or H, X2 is absent or D or G, X3 is D or V or I or S or M, and X5 is any amino acid. In some embodiments, this sequence comprises a non-conserved sequence of amino-acids with a strong alpha-helical propensity followed by the consensus sequence -D(V/I)ExNPG P, where x= any amino acid. Some nonlimiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the linear polyribonucleotide includes a termination sequence at the end of one or more expression sequences. In some embodiments, one or more expression sequences lacks a termination sequence. Generally, termination sequences include an in-frame nucleotide triplet that signals termination of translation, e.g., UAA, UGA, UAG. In some embodiments, one or more termination sequences in the linear polyribonucleotide are frame-shifted termination sequences, such as but not limited to, off-frame or -1 and +1 shifted reading frames (e.g., hidden stop) that can terminate translation. Frame-shifted termination sequences include nucleotide triples, TAA, TAG, and TGA that appear in the second and third reading frames of an expression sequence. Frame-shifted termination sequences can be important in preventing misreads of mRNA, which is often detrimental to the cell.

In some embodiments, a stagger sequence described herein can terminate translation and/or cleave an expression product between G and P of the consensus sequence described herein. As one non-limiting example, the linear polyribonucleotide includes at least one stagger sequence to terminate translation and/or cleave the expression product. In some embodiments, the linear polyribonucleotide includes a stagger sequence adjacent to at least one expression sequence. In some embodiments, the linear polyribonucleotide includes a stagger sequence after each expression sequence. In some embodiments, the linear polyribonucleotide includes a stagger sequence is present on one or both sides of each expression sequence, leading to translation of individual peptide(s) and or polypeptide(s) from each expression sequence.

### PolyA Sequence

In some embodiments, the linear polyribonucleotide includes a poly-A sequence. In some embodiments, the length of a poly-A sequence is greater than 10 nucleotides in length. In some embodiments, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A sequence is designed relative to the length of the overall linear polyribonucleotide. The design can be based on the length of the coding region, the length of a particular feature or region (such as the first or flanking regions), or based on the length of the ultimate product expressed from the linear polyribonucleotide. In this context, the poly-A sequence can be 10, 20, 30, 40, 50, 60, 70, 80, 90, or 100% greater in length than the linear polyribonucleotide or a feature thereof. The poly-A sequence can also be designed as a fraction of the linear polyribonucleotide. In this context, the poly-A sequence can be 10%, 20%, 30%, 40%, 50%, 60%, 70%, 80%, 90%, or more of the total length of the construct or the total length of the construct minus the poly-A sequence. Further, engineered binding sites and conjugation of linear polyribonucleotide for Poly-A binding protein can enhance expression.

In some embodiments, the linear polyribonucleotide is designed to include a polyA-G quartet. The G-quartet is a cyclic hydrogen bonded array of four guanine nucleotides that can be formed by G-rich sequences in both DNA and RNA. In some embodiments, the G-quartet can be incorporated at the end of the poly-A sequence. The resultant linear polyribonucleotide construct can be assayed for stability, protein production, and/or other parameters including half-life at various time points. In some embodiments, the polyA-G quartet can result in protein production equivalent to at least 75% of that seen using a poly-A sequence of 120 nucleotides alone.

### Riboswitches

In some embodiments, the linear polyribonucleotide comprises one or more riboswitches.

A riboswitch can be a part of the linear polyribonucleotide that can directly bind a small target molecule, and whose binding of the target affects RNA translation and the expression product stability and activity. Thus, the linear polyribonucleotide that includes a riboswitch can regulate the activity of the linear polyribonucleotide depending on the presence or absence of the target molecule. In some embodiments, a riboswitch has a region of aptamer-like affinity for a separate molecule. Any aptamer included within a non-coding nucleic acid can be used for sequestration of molecules from bulk volumes. In some embodiments, “(ribo)switch” activity can be used for downstream reporting of the event.

In some embodiments, the riboswitch modulates gene expression by transcriptional termination, inhibition of translation initiation, mRNA self-cleavage, and in eukaryotes, alteration of splicing pathways. The riboswitch can control gene expression through the binding or removal of a trigger molecule. Thus, subjecting a linear polyribonucleotide that includes the riboswitch to conditions that activate, deactivate, or block the riboswitch can alter gene expression. For example, gene expression can be altered as a result of termination of transcription or blocking of ribosome binding to the RNA. Binding of a trigger molecule, or an analog thereof, can reduce/prevent expression or promote/increase expression of the RNA molecule depending on the nature of the riboswitch.

In some embodiments, the riboswitch is a Cobalamin riboswitch (also B12-e1ement), which binds adenosylcobalamin (the coenzyme form of vitamin B12) to regulate the biosynthesis and transport of cobalamin and similar metabolites.

In some embodiments, the riboswitch is a cyclic di-GMP riboswitch, which binds cyclic di-GMP to regulate a variety of genes. There are two non-structurally related classes of cyclic di-GMP riboswitch: cyclic di-GMP-I and cyclic di-GMP-II.

In some embodiments, the riboswitch is a FMN riboswitch (also RFN-element) which binds flavin mononucleotide (FMN) to regulate riboflavin biosynthesis and transport.

In some embodiments, the riboswitch is a glmS riboswitch, which cleaves itself when there is a sufficient concentration of glucosamine-6-phosphate.

In some embodiments, the riboswitch is a glutamine riboswitch, which binds glutamine to regulate genes involved in glutamine and nitrogen metabolism. Glutamine riboswitches can also bind short peptides of unknown function. Such riboswitches fall into two structurally related classes: the glnA RNA motif and Downstream-peptide motif.

In some embodiments, the riboswitch is a glycine riboswitch, which binds glycine to regulate glycine metabolism genes. It comprises two adjacent aptamer domains in the same mRNA, and is the only known natural RNA that exhibits cooperative binding.

In some embodiments, the riboswitch is a lysine riboswitch (also L-box), which binds lysine to regulate lysine biosynthesis, catabolism, and transport.

In some embodiments, the riboswitch is a preQ1 riboswitch, which binds pre-queuosine to regulate genes involved in the synthesis or transport of this precursor to queuosine. Two distinct classes of preQ1 riboswitches are preQ1-I riboswitches and preQ1-II riboswitches. The binding domain of preQ1 -I riboswitches is unusually small among naturally occurring riboswitches. PreQl-II riboswitches, which are only found in certain species in the genera Streptococcus and Lactococcus, have a completely different structure and are larger than preQ1-I riboswitches.

In some embodiments, the riboswitch is a purine riboswitch, which binds purines to regulate purine metabolism and transport. Different forms of purine riboswitches bind guanine or adenine. The specificity for either guanine or adenine depends upon Watson-Crick interactions with a single pyrimidine in the riboswitch at position Y74. In the guanine riboswitch, the single pyrimidine is cytosine (i.e. C74). In the adenine riboswitch, the single pyrimidine is uracil (i.e. U74). Homologous types of purine riboswitches can bind deoxyguanosine, but have more significant differences than a single nucleotide mutation.

In some embodiments, the riboswitch is an S-adenosylhomocysteine (SAH) riboswitch, which binds SAH to regulate genes involved in recycling SAH produced from S-adenosylmethionine (SAM) in methylation reactions.

In some embodiments, the riboswitch is an S-adenosyl methionine (SAM) riboswitch, which binds SAM to regulate methionine and SAM biosynthesis and transport. There are three distinct SAM riboswitches: SAM-I (originally called S-box), SAM-II, and the SMK box. SAM-I is widespread in bacteria. SAM-II is found only in α-, β-, and a few y-proteobacteria. The SMK box riboswitch is found in Lactobacillales. These three varieties of riboswitch have no obvious sequence or structural similarities. A fourth variety, SAM-IV, appears to have a similar ligand-binding core to that of SAM-I, but in the context of a distinct scaffold.

In some embodiments, the riboswitch is a SAM-SAH riboswitch, which binds both SAM and SAH with similar affinities.

In some embodiments, the riboswitch is a tetrahydrofolate riboswitch, which binds tetrahydrofolate to regulate synthesis and transport genes.

In some embodiments, the riboswitch is a theophylline-binding riboswitch or a thymine pyrophosphate-binding riboswitch.

In some embodiments, the riboswitch is a glmS catalytic riboswitch from Thermoanaerobacter tengcongensis, which senses glucosamine-6 phosphate.

In some embodiments, the riboswitch is a thiamine pyrophosphate (TPP) riboswitch (also Thi-box), which binds TPP to regulate thiamine biosynthesis and transport, as well as transport of similar metabolites. The TPP riboswitch is found in eukaryotes.

In some embodiments, the riboswitch is a Moco riboswitch, which binds molybdenum cofactor, to regulate genes involved in biosynthesis and transport of this coenzyme, as well as enzymes that use molybdenum or derivatives thereof as a cofactor.

In some embodiments, the riboswitch is an adenine-sensing add-A riboswitch, found in the 5′-UTR of the adenine deaminase (add) encoding gene of Vibrio vulnificus.

### Aptazyme

In some embodiments, the linear polyribonucleotide comprises an aptazyme. Aptazyme is a switch for conditional expression in which an aptamer region is used as an allosteric control element and coupled to a region of catalytic RNA (a “ribozyme” as described below). In some embodiments, the aptazyme is active in cell type-specific translation. In some embodiments, the aptazyme is active under cell state-specific translation, e.g., virally infected cells or in the presence of viral nucleic acids or viral proteins.

A ribozyme is a RNA molecule that catalyzes a chemical reaction. Many natural ribozymes can catalyze the hydrolysis of phosphodiester bonds of the ribozyme itself or the hydrolysis of phosphodiester bonds in other RNA. Natural ribozymes can also catalyze the aminotransferase activity of the ribosome. Catalytic RNA can be “evolved” by in vitro methods. Ribozymes and reaction products of ribozymes can regulate gene expression. In some embodiments, a catalytic RNA or ribozyme can be placed within a larger, non-coding RNA such that the ribozyme is present at many copies within the cell for chemical transformation of a molecule from a bulk volume. In some embodiments, aptamers and ribozymes can both be encoded in the same non-coding RNA.

Non-limiting examples of ribozymes include hammerhead ribozyme, VL ribozyme, leadzyme, and hairpin ribozyme.

In some embodiments, the aptazyme is a ribozyme that can cleave RNA sequences and can be regulated as a result of binding a ligand or modulator. The ribozyme can be a self-cleaving ribozyme. As such, these ribozymes can combine the properties of ribozymes and aptamers.

In some embodiments, the aptazyme is included in an untranslated region of linear polyribonucleotides described herein. An aptazyme in the absence of ligand/modulator is inactive, which can allow expression of the transgene. Expression can be turned off or down-regulated by addition of the ligand. Aptazymes that are downregulated in response to the presence of a particular modulator can be used in control systems where upregulation of gene expression in response to modulator is desired.

Aptazymes can also be used to develop of systems for self-regulation of linear polyribonucleotide expression. For example, the protein product of linear polyribonucleotides described herein that is the rate determining enzyme in the synthesis of a particular small molecule can be modified to include an aptazyme that is selected to have increased catalytic activity in the presence of the small molecule to provide an autoregulatory feedback loop for synthesis of the molecule. Alternatively, the aptazyme activity can be selected sense accumulation of the protein product from the linear polyribonucleotide, or any other cellular macromolecule.

In some embodiments, the linear polyribonucleotide can include an aptamer sequence. Non-limiting examples of aptamers include RNA aptamers that bind lysozyme, Toggle-25t (an RNA aptamer containing 2′-fluoropyrimidine nucleotides that binds thrombin with high specificity and affinity), RNA-Tat that binds human immunodeficiency virus trans-acting responsive element (HIV TAR), RNA aptamers that bind hemin, RNA aptamers that bind interferon γ, RNA aptamer binding vascular endothelial growth factor (VEGF), RNA aptamers that bind prostate specific antigen (PSA), RNA aptamers that bind dopamine, and RNA aptamers that bind heat shock factor 1 (HSF1).

In some embodiments, linear RNA described herein can be used for transcription and replication of RNA. For example, linear RNA can be used to encode non-coding RNA, lncRNA, miRNA, tRNA, rRNA, snoRNA, ncRNA, siRNA, or shRNA. In some embodiments, linear RNA can include anti-sense miRNA and a transcriptional element. After transcription, such linear RNA can produce functional, linear miRNAs. Non-limiting examples of linear RNA expression and modulation applications are listed in TABLE 3.

TABLE 3

| | |
|---|---|
| Process | MOA (example) |
| Combinatoional therapy of inhibition & translation | Inhibition of one protein and supplementation of another (or same) |

### Replication Element

The linear polyribonucleotide can encode a sequence and/or motif useful for replication. Replication of a linear polyribonucleotide can occur by generating a complement linear polyribonucleotide. In some embodiments, the linear polyribonucleotide includes a motif to initiate transcription, where transcription is driven by either endogenous cellular machinery (DNA-dependent RNA polymerase) or an RNA-depended RNA polymerase encoded by the linear polyribonucleotide. The ribozymes can be encoded by the linear polyribonucleotide, its complement, or by an RNA sequence in trans. In some embodiments, the encoded ribozymes can include a sequence or motif that regulates (inhibits or promotes) activity of the ribozyme to control linear RNA propagation. In some embodiments, unit-length sequences can be ligated into a linear form by a cellular RNA ligase. In some embodiments, the linear polyribonucleotide includes a replication element that aids in self-amplification. Examples of such replication elements include HDV replication domains and replication competent linear RNA sense and/or antisense ribozymes, such as antigenomic 5′-CGGGUCGGCAUGGCAUCUCCACCUCCUCGC GGUCCGACCUGGGCAUCCGAAGGAGGACGCACGUCCACUCGGAUGGCUAAGGGAGAGCC A-3′ or genomic 5′-UGGCCGGCAUGGUCCCAGCCUCCUCGCUGGCGCCGGCUGGGCAACAUU CCGAGGGGACCGUCCCCUCGGUAAUGGCGAAUGGGACCCA-3′.

In some embodiments, the linear polyribonucleotide includes at least one cleavage sequence as described herein to aid in replication. A cleavage sequence within the linear polyribonucleotide can cleave long transcripts replicated from the linear polyribonucleotide to a specific length that can subsequently linearize to form a complement to the linear polyribonucleotide.

In another embodiment, the linear polyribonucleotide includes at least one ribozyme sequence to cleave long transcripts replicated from the linear polyribonucleotide to a specific length, where another encoded ribozyme cuts the transcripts at the ribozyme sequence. Linearization forms a complement to the linear polyribonucleotide.

In some embodiments, the linear polyribonucleotide is substantially resistant to degradation, e.g., by exonucleases, when the 5′ and/or 3′ end is modified, e.g., by an end protectant.

In some embodiments, the linear polyribonucleotide replicates within a cell. In some embodiments, the linear polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage there between. In some embodiments, the linear polyribonucleotide is replicates within a cell and is passed to daughter cells. In some embodiments, a cell passes at least one linear polyribonucleotide to daughter cells with an efficiency of at least 25%, 50%, 60%, 70%, 80%, 85%, 90%, 95%, or 99%. In some embodiments, cell undergoing meiosis passes the linear polyribonucleotide to daughter cells with an efficiency of at least 25%, 50%, 60%, 70%, 80%, 85%, 90%, 95%, or 99%. In some embodiments, a cell undergoing mitosis passes the linear polyribonucleotide to daughter cells with an efficiency of at least 25%, 50%, 60%, 70%, 80%, 85%, 90%, 95%, or 99%.

In some embodiments, the linear polyribonucleotide replicates within the host cell. In some embodiments, the linear polyribonucleotide is capable of replicating in a mammalian cell, e.g., human cell.

While in some embodiments the linear polyribonucleotide replicates in the host cell, the linear polyribonucleotide does not integrate into the genome of the host, e.g., with the host’s chromosomes. In some embodiments, the linear polyribonucleotide has a negligible recombination frequency, e.g., with the host’s chromosomes. In some embodiments, the linear polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host’s chromosomes.

### Other Sequences

In some embodiments, the linear polyribonucleotide further includes another nucleic acid sequence. In some embodiments, the linear polyribonucleotide can include DNA, RNA, or artificial nucleic acid sequences. The other sequences can include, but are not limited to, genomic DNA, cDNA, or sequences that encode tRNA, mRNA, rRNA, miRNA, gRNA, siRNA, or other RNAi molecules. In some embodiments, the linear polyribonucleotide includes a sequence encoding an siRNA to target a different locus or loci of the same gene expression product as the linear polyribonucleotide. In some embodiments, the linear polyribonucleotide includes a sequence encoding an siRNA to target a different gene expression product as the linear polyribonucleotide.

In some embodiments, the linear polyribonucleotide lacks a 5′-UTR. In some embodiments, the linear polyribonucleotide lacks a 3′-UTR. In some embodiments, the linear polyribonucleotide lacks a poly-A sequence. In some embodiments, the linear polyribonucleotide lacks a termination sequence. In some embodiments, the linear polyribonucleotide lacks an internal ribosomal entry site. In some embodiments, the linear polyribonucleotide lacks degradation susceptibility by exonucleases. In some embodiments, the linear polyribonucleotide lacks binding to cap-binding proteins. In some embodiments, the linear polyribonucleotide lacks a 5′ cap.

In some embodiments, the linear polyribonucleotide comprises one or more of the following sequences: a sequence that encodes one or more miRNA, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNA, shRNA), and a sequence that encodes a therapeutic mRNA or protein.

The other sequence can have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range there between.

### Nucleotide Spacer Sequences

In some embodiments, the linear polyribonucleotide comprises a spacer sequence.

The spacer can be a nucleic acid molecule having low GC content, for example less than 65%, 60%, 55%, 50%, 55%, 50%, 45%, 40%, 39%, 38%, 37%, 36%, 35%, 34%, 33%, 32%, 31 %, 30%, 29%, 28%, 27%, 26%, 25%, 24%, 23%, 22%, 20%, 19%, 18%, 17%, 1 6%, 15%, 14%, 13%, 12%, 1 1 %, 10%, 9%, 8%, 7%, 6%, 5%, 4%, 3%, 2% or 1 %, across the full length of the spacer, or across at least 50%, 60%, 70%, 80%, 90%, 91 %, 92%, 93%, 94%, 95%, 96%, 97%, 98% or 99% contiguous nucleic acid residues of the spacer. In some embodiments, the spacer is substantially free of a secondary structure, such as less than 40 kcal/mol, less than -39, -38, -37, -36, -35, -34, -33, -32, -31, -30, -29, -28, -27, -26, -25, -24, -23, -22, -20, -19, -18, -17, -16, -15, -14, -13, -12, -11, -10, -9, -8, -7, -6, -5, -4, -3, -2 or -1 kcal/mol. The spacer can include a nucleic acid, such as DNA or RNA.

The spacer sequence can encode an RNA sequence, and preferably a protein or peptide sequence, including a secretion signal peptide.

The spacer sequence can be non-coding. Where the spacer is a non-coding sequence, a start codon can be provided in the coding sequence of an adjacent sequence. In some embodiments, it is envisaged that the first nucleic acid residue of the coding sequence can be the A residue of a start codon, such as AUG. Where the spacer encodes an RNA or protein or peptide sequence, a start codon can be provided in the spacer sequence.

In some embodiments, the spacer is operably linked to another sequence described herein.

### Non-Nucleic Acid Linkers

The linear polyribonucleotide described herein can also comprise a non-nucleic acid linker. In some embodiments, the linear polyribonucleotide described herein has a non-nucleic acid linker between one or more of the sequences or elements described herein. In some embodiments, one or more sequences or elements described herein are linked with the linker. The non-nucleic acid linker can be a chemical bond, e.g., one or more covalent bonds or non-covalent bonds. In some embodiments, the non-nucleic acid linker is a peptide or protein linker. Such a linker can be between 2-30 amino acids, or longer. The linker includes flexible, rigid or cleavable linkers described herein.

The most commonly used flexible linkers have sequences consisting primarily of stretches of Gly and Ser residues (“GS” linker). Flexible linkers can be useful for joining domains that require a certain degree of movement or interaction and can include small, non-polar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers can also be useful when a spatial separation of the domains is critical to preserve the stability or bioactivity of one or more components in the fusion. Rigid linkers can have an alpha helix-structure or Pro-rich sequence, (XP)ₙ, with X designating any amino acid, preferably Ala, Lys, or Glu.

Cleavable linkers can release free functional domains in vivo. In some embodiments, linkers can be cleaved under specific conditions, such as the presence of reducing reagents or proteases. In vivo cleavable linkers can utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. In vivo cleavage of linkers in fusions can also be carried out by proteases that are expressed in vivo under pathological conditions (e.g., cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (-CH₂-ipids, such as a poly (-CHe g polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide can be linked using charge-based chemistry, such that a positively charged moiety of the polypeptide is linked to a negative charge of another polypeptide or nucleic acid.

### Structure

In some embodiments, the linear polyribonucleotide comprises a higher order structure, e.g., a secondary or tertiary structure. In some embodiments, complementary segments of the linear polyribonucleotide fold itself into a double stranded segment, held together with hydrogen bonds between pairs, e.g., A-U and C-G. In some embodiments, helices, also known as stems, are formed intramolecularly, having a double-stranded segment connected to an end loop. In some embodiments, the linear polyribonucleotide has at least one segment with a quasi-double-stranded secondary structure. In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, a segment having a quasi-double-stranded secondary structure has 3 paired nucleotides. In some embodiments, a segment having a quasi-double-stranded secondary structure has 4 paired nucleotides. In some embodiments, a segment having a quasi-double-stranded secondary structure has 5 paired nucleotides. In some embodiments, a segment having a quasi-double-stranded secondary structure has 6 paired nucleotides. In some embodiments, a segment having a quasi-double-stranded secondary structure has 7 paired nucleotides. In some embodiments, a segment having a quasi-double-stranded secondary structure has 8 paired nucleotides. In some embodiments, a segment having a quasi-double-stranded secondary structure has 9 paired nucleotides. In some embodiments, a segment having a quasi-double-stranded secondary structure has 10 paired nucleotides. In some embodiments, a segment having a quasi-double-stranded secondary structure has 11 paired nucleotides. In some embodiments, a segment having a quasi-double-stranded secondary structure has 12 paired nucleotides. In some embodiments, a segment having a quasi-double-stranded secondary structure has 13 paired nucleotides. In some embodiments, a segment having a quasi-double-stranded secondary structure has 14 paired nucleotides. In some embodiments, a segment having a quasi-double-stranded secondary structure has 15 paired nucleotides. In some embodiments, a segment having a quasi-double-stranded secondary structure has 16 paired nucleotides. In some embodiments, a segment having a quasi-double-stranded secondary structure has 17 paired nucleotides. In some embodiments, a segment having a quasi-double-stranded secondary structure has 18 paired nucleotides. In some embodiments, a segment having a quasi-double-stranded secondary structure has 19 paired nucleotides. In some embodiments, a segment having a quasi-double-stranded secondary structure has 20 paired nucleotides. In some embodiments, a segment having a quasi-double-stranded secondary structure has 21 paired nucleotides. In some embodiments, a segment having a quasi-double-stranded secondary structure has 22 paired nucleotides. In some embodiments, a segment having a quasi-double-stranded secondary structure has 23 paired nucleotides. In some embodiments, a segment having a quasi-double-stranded secondary structure has 24 paired nucleotides. In some embodiments, a segment having a quasi-double-stranded secondary structure has 25 paired nucleotides. In some embodiments, a segment having a quasi-double-stranded secondary structure has 26 paired nucleotides. In some embodiments, a segment having a quasi-double-stranded secondary structure has 27 paired nucleotides. In some embodiments, a segment having a quasi-double-stranded secondary structure has 28 paired nucleotides. In some embodiments, a segment having a quasi-double-stranded secondary structure has 29 paired nucleotides. In some embodiments, a segment having a quasi-double-stranded secondary structure has 30 paired nucleotides. In some embodiments, a segment having a quasi-double-stranded secondary structure has 35 paired nucleotides. In some embodiments, a segment having a quasi-double-stranded secondary structure has 40 paired nucleotides. In some embodiments, a segment having a quasi-double-stranded secondary structure has 45 paired nucleotides. In some embodiments, a segment having a quasi-double-stranded secondary structure has 50 paired nucleotides. In some embodiments, a segment having a quasi-double-stranded secondary structure has 55 paired nucleotides. In some embodiments, a segment having a quasi-double-stranded secondary structure has 60 paired nucleotides. In some embodiments, a segment having a quasi-double-stranded secondary structure has 65 paired nucleotides. In some embodiments, a segment having a quasi-double-stranded secondary structure has 70 paired nucleotides. In some embodiments, a segment having a quasi-double-stranded secondary structure has 75 paired nucleotides. In some embodiments, a segment having a quasi-double-stranded secondary structure has 80 paired nucleotides. In some embodiments, a segment having a quasi-double-stranded secondary structure has 85 paired nucleotides. In some embodiments, a segment having a quasi-double-stranded secondary structure has 90 paired nucleotides. In some embodiments, a segment having a quasi-double-stranded secondary structure has 95 paired nucleotides. In some embodiments, a segment having a quasi-double-stranded secondary structure has 100 paired nucleotides. In some embodiments, the linear polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the segments are separated by 3 nucleotides. In some embodiments, the segments are separated by 4 nucleotides. In some embodiments, the segments are separated by 5 nucleotides. In some embodiments, the segments are separated by 6 nucleotides. In some embodiments, the segments are separated by 7 nucleotides. In some embodiments, the segments are separated by 8 nucleotides. In some embodiments, the segments are separated by 9 nucleotides. In some embodiments, the segments are separated by 10 nucleotides. In some embodiments, the segments are separated by 11 nucleotides. In some embodiments, the segments are separated by 12 nucleotides. In some embodiments, the segments are separated by 13 nucleotides. In some embodiments, the segments are separated by 14 nucleotides. In some embodiments, the segments are separated by 15 nucleotides. In some embodiments, the segments are separated by 16 nucleotides. In some embodiments, the segments are separated by 17 nucleotides. In some embodiments, the segments are separated by 18 nucleotides. In some embodiments, the segments are separated by 19 nucleotides. In some embodiments, the segments are separated by 20 nucleotides. In some embodiments, the segments are separated by 21 nucleotides. In some embodiments, the segments are separated by 22 nucleotides. In some embodiments, the segments are separated by 23 nucleotides. In some embodiments, the segments are separated by 24 nucleotides. In some embodiments, the segments are separated by 25 nucleotides. In some embodiments, the segments are separated by 26 nucleotides. In some embodiments, the segments are separated by 27 nucleotides. In some embodiments, the segments are separated by 28 nucleotides. In some embodiments, the segments are separated by 29 nucleotides. In some embodiments, the segments are separated by 30 nucleotides. In some embodiments, the segments are separated by 35 nucleotides. In some embodiments, the segments are separated by 40 nucleotides. In some embodiments, the segments are separated by 45 nucleotides. In some embodiments, the segments are separated by 50 nucleotides. In some embodiments, the segments are separated by 55 nucleotides. In some embodiments, the segments are separated by 60 nucleotides. In some embodiments, the segments are separated by 65 nucleotides. In some embodiments, the segments are separated by 70 nucleotides. In some embodiments, the segments are separated by 75 nucleotides. In some embodiments, the segments are separated by 80 nucleotides. In some embodiments, the segments are separated by 85 nucleotides. In some embodiments, the segments are separated by 90 nucleotides. In some embodiments, the segments are separated by 95 nucleotides. In some embodiments, the segments are separated by 100 nucleotides.

There are 16 possible base-pairings, however of these, six (AU, GU, GC, UA, UG, CG) can form actual base-pairs. The rest are called mismatches and occur at very low frequencies in helices. In some embodiments, the structure of the linear polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the linear polyribonucleotide has a quasi-helical structure. In some embodiments, the linear polyribonucleotide has at least one segment with a quasi-helical structure. In some embodiments, a segment having a quasi-helical structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, a segment having a quasi-helical structure has 3 nucleotides. In some embodiments, a segment having a quasi-helical structure has 4 nucleotides. In some embodiments, a segment having a quasi-helical structure has 5 nucleotides. In some embodiments, a segment having a quasi-helical structure has 6 nucleotides. In some embodiments, a segment having a quasi-helical structure has 7 nucleotides. In some embodiments, a segment having a quasi-helical structure has 8 nucleotides. In some embodiments, a segment having a quasi-helical structure has 9 nucleotides. In some embodiments, a segment having a quasi-helical structure has 10 nucleotides. In some embodiments, a segment having a quasi-helical structure has 11 nucleotides. In some embodiments, a segment having a quasi-helical structure has 12 nucleotides. In some embodiments, a segment having a quasi-helical structure has 13 nucleotides. In some embodiments, a segment having a quasi-helical structure has 14 nucleotides. In some embodiments, a segment having a quasi-helical structure has 15 nucleotides. In some embodiments, a segment having a quasi-helical structure has 16 nucleotides. In some embodiments, a segment having a quasi-helical structure has 17 nucleotides. In some embodiments, a segment having a quasi-helical structure has 18 nucleotides. In some embodiments, a segment having a quasi-helical structure has 19 nucleotides. In some embodiments, a segment having a quasi-helical structure has 20 nucleotides. In some embodiments, a segment having a quasi-helical structure has 21 nucleotides. In some embodiments, a segment having a quasi-helical structure has 22 nucleotides. In some embodiments, a segment having a quasi-helical structure has 23 nucleotides. In some embodiments, a segment having a quasi-helical structure has 24 nucleotides. In some embodiments, a segment having a quasi-helical structure has 25 nucleotides. In some embodiments, a segment having a quasi-helical structure has 26 nucleotides. In some embodiments, a segment having a quasi-helical structure has 27 nucleotides. In some embodiments, a segment having a quasi-helical structure has 28 nucleotides. In some embodiments, a segment having a quasi-helical structure has 29 nucleotides. In some embodiments, a segment having a quasi-helical structure has 30 nucleotides. In some embodiments, a segment having a quasi-helical structure has 35 nucleotides. In some embodiments, a segment having a quasi-helical structure has 40 nucleotides. In some embodiments, a segment having a quasi-helical structure has 45 nucleotides. In some embodiments, a segment having a quasi-helical structure has 50 nucleotides. In some embodiments, a segment having a quasi-helical structure has 55 nucleotides. In some embodiments, a segment having a quasi-helical structure has 60 nucleotides. In some embodiments, a segment having a quasi-helical structure has 65 nucleotides. In some embodiments, a segment having a quasi-helical structure has 70 nucleotides. In some embodiments, a segment having a quasi-helical structure has 75 nucleotides. In some embodiments, a segment having a quasi-helical structure has 80 nucleotides. In some embodiments, a segment having a quasi-helical structure has 85 nucleotides. In some embodiments, a segment having a quasi-helical structure has 90 nucleotides. In some embodiments, a segment having a quasi-helical structure has 95 nucleotides. In some embodiments, a segment having a quasi-helical structure has 100 nucleotides. In some embodiments, the linear polyribonucleotide has one or more segments (e.g., 2, 3, 4, 5, 6, or more) having a quasi-helical structure. In some embodiments, the linear polyribonucleotide has one segment having a quasi-helical structure. In some embodiments, the linear polyribonucleotide has 2 segments having a quasi-helical structure. In some embodiments, the linear polyribonucleotide has 3 segments having a quasi-helical structure. In some embodiments, the linear polyribonucleotide has 4 segments having a quasi-helical structure. In some embodiments, the linear polyribonucleotide has 5 segments having a quasi-helical structure. In some embodiments, the linear polyribonucleotide has 6 segements. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the segments are separated by 3 nucleotides. In some embodiments, the segments are separated by 4 nucleotides. In some embodiments, the segments are separated by 5 nucleotides. In some embodiments, the segments are separated by 6 nucleotides. In some embodiments, the segments are separated by 7 nucleotides. In some embodiments, the segments are separated by 8 nucleotides. In some embodiments, the segments are separated by 9 nucleotides. In some embodiments, the segments are separated by 10 nucleotides. In some embodiments, the segments are separated by 11 nucleotides. In some embodiments, the segments are separated by 12 nucleotides. In some embodiments, the segments are separated by 13 nucleotides. In some embodiments, the segments are separated by 14 nucleotides. In some embodiments, the segments are separated by 15 nucleotides. In some embodiments, the segments are separated by 16 nucleotides. In some embodiments, the segments are separated by 17 nucleotides. In some embodiments, the segments are separated by 18 nucleotides. In some embodiments, the segments are separated by 19 nucleotides. In some embodiments, the segments are separated by 20 nucleotides. In some embodiments, the segments are separated by 21 nucleotides. In some embodiments, the segments are separated by 22 nucleotides. In some embodiments, the segments are separated by 23 nucleotides. In some embodiments, the segments are separated by 24 nucleotides. In some embodiments, the segments are separated by 25 nucleotides. In some embodiments, the segments are separated by 26 nucleotides. In some embodiments, the segments are separated by 27 nucleotides. In some embodiments, the segments are separated by 28 nucleotides. In some embodiments, the segments are separated by 29 nucleotides. In some embodiments, the segments are separated by 30 nucleotides. In some embodiments, the segments are separated by 35 nucleotides. In some embodiments, the segments are separated by 40 nucleotides. In some embodiments, the segments are separated by 45 nucleotides. In some embodiments, the segments are separated by 50 nucleotides. In some embodiments, the segments are separated by 55 nucleotides. In some embodiments, the segments are separated by 60 nucleotides. In some embodiments, the segments are separated by 65 nucleotides. In some embodiments, the segments are separated by 70 nucleotides. In some embodiments, the segments are separated by 75 nucleotides. In some embodiments, the segments are separated by 80 nucleotides. In some embodiments, the segments are separated by 85 nucleotides. In some embodiments, the segments are separated by 90 nucleotides. In some embodiments, the segments are separated by 95 nucleotides. In some embodiments, the segments are separated by 100 nucleotides. In some embodiments, the linear polyribonucleotide includes at least one of a U-rich or A-rich sequence or a combination thereof. In some embodiments, the U-rich and/or A-rich sequences are arranged in a manner that would produce a triple quasi-helix structure. In some embodiments, the linear polyribonucleotide has a double quasi-helical structure. In some embodiments, the linear polyribonucleotide has one or more segments (e.g., 2, 3, 4, 5, 6, or more) having a double quasi-helical structure. In some embodiments, the linear polyribonucleotide includes at least one of a C-rich and/or G-rich sequence. In some embodiments, the C-rich and/or G-rich sequences are arranged in a manner that would produce triple quasi-helix structure. In some embodiments, the linear polyribonucleotide has an intramolecular triple quasi-helix structure that aids in stabilization.

In some embodiments, the linear polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the linear polyribonucleotide has at least one miRNA binding site, at least one lncRNA binding site, and/or at least one tRNA motif.

### Methods of Production

In some embodiments, the linear polyribonucleotide disclosed herein includes a deoxyribonucleic acid sequence that is non-naturally occurring and can be produced using recombinant DNA technology or chemical synthesis.

It is within the scope of the invention that a DNA molecule used to produce an RNA strand can comprise a DNA sequence of a naturally-occurring original nucleic acid sequence, a modified version thereof, or a DNA sequence encoding a synthetic polypeptide not normally found in nature (e.g., chimeric molecules or fusion proteins). DNA molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

Linear polyribonucleotides may be prepared according to any available technique including, but not limited to chemical synthesis; enzymatic synthesis, which is generally termed in vitro transcription (IVT); or enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005.)

In some embodiments, the synthesis of linear polyribonucleotides involves polyribonucleotide production (either with or without modifications) and purification. In the enzymatic synthesis method, a polynucleotide sequence encoding the gene of interest incorporated into a vector which will be amplified to produce a cDNA template. The cDNA template is then used to produce RNA through in vitro transcription (IVT). In some embodiments, the template is a linear RNA strand. After production, the RNA may undergo purification and clean-up processes. The steps of which are provided in more detail below.

### Polynucleotide Production

The process of polynucleotide production may include, but is not limited to, in vitro transcription, cDNA template removal and RNA clean-up, and RNA capping and/or tailing reactions. Alternatively, the synthetic polynucleotide can be chemically synthesized.

### In Vitro Transcription

The cDNA produced in the previous step may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids. Inorganic pyrophosphatase can be included in the transcription system.

### RNA Polymerases

Any number of RNA polymerases or variants may be used in the design of the primary constructs of the present invention. In some embodimetns, the RNA polymerase can use DNA or RNA as a template.

RNA polymerases may be modified by inserting or deleting amino acids of the RNA polymerase sequence. As a non-limiting example, the RNA polymerase may be modified to exhibit an increased ability to incorporate a 2′-modified nucleotide triphosphate compared to an unmodified RNA polymerase (see International Publication WO2008078180 and U.S. Pat. No. 8,101,385).

In some embodiments, the linear polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the linear polyribonucleotide is 20 nucleotides. In some embodiments, the linear polyribonucleotide is 30 nucleotides. In some embodiments, the linear polyribonucleotide is 40 nucleotides. In some embodiments, the linear polyribonucleotide is 50 nucleotides. In some embodiments, the linear polyribonucleotide is 75 nucleotides. In some embodiments, the linear polyribonucleotide is 100 nucleotides. In some embodiments, the linear polyribonucleotide is 200 nucleotides. In some embodiments, the linear polyribonucleotide is 300 nucleotides. In some embodiments, the linear polyribonucleotide is 400 nucleotides. In some embodiments, the linear polyribonucleotide is 500 nucleotides. In some embodiments, the linear polyribonucleotide is 1,000 nucleotides. In some embodiments, the linear polyribonucleotide is 2,000 nucleotides. In some embodiments, the linear polyribonucleotide is 5,000 nucleotides. In some embodiments, the linear polyribonucleotide is 6,000 nucleotides. In some embodiments, the linear polyribonucleotide is 7,000 nucleotides. In some embodiments, the linear polyribonucleotide is 8,000 nucleotides. In some embodiments, the linear polyribonucleotide is 9,000 nucleotides. In some embodiments, the linear polyribonucleotide is 10,000 nucleotides. In some embodiments, the linear polyribonucleotide is 12,000 nucleotides. In some embodiments, the linear polyribonucleotide is 14,000 nucleotides. In some embodiments, the linear polyribonucleotide is 15,000 nucleotides. In some embodiments, the linear polyribonucleotide is 16,000 nucleotides. In some embodiments, the linear polyribonucleotide is 17,000 nucleotides. In some embodiments, the linear polyribonucleotide is 18,000 nucleotides. In some embodiments, the linear polyribonucleotide is 19,000 nucleotides. In some embodiments, the linear polyribonucleotide is 20,000 nucleotides. In some embodiments, the linear polyribonucleotide may be of a sufficient size to accommodate a binding site for a ribosome. One of skill in the art can appreciate that the maximum size of a linear polyribonucleotide can be as large as is within the technical constraints of producing a linear polyribonucleotide, and/or using the linear polyribonucleotide. While not being bound by theory, it is possible that multiple segments of RNA may be produced from DNA and their 5′ and 3′ free ends annealed to produce a “string” of RNA, which can form the linear polyribonucleotide. In some embodiments, the maximum size of a linear polyribonucleotide may be limited by the ability of packaging and delivering the RNA to a target. In some embodiments, the size of a linear polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least at 400 nucleotides, at least 300 nucleotides, at least 200 nucleotides, at least 100 nucleotides may be useful.

In some embodiments, the linear polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof.

### Chemical Compound

A linear RNA disclosed herein can be conjugated to a chemical compound by a conjugation moiety. A chemical compound can recruit a target or a substrate. The target can be a target protein. The substrate can be substrate protein of the target protein. The chemical compound can be a target protein ligand. A chemial compound can be a molecule selected for its ability to interact with a collection of functional groups.

A chemical compound can be a small molecule. A chemical compound can bind to a substrate protein, such as compound that binds to Human BET Bromodomain-containing proteins, the aryl hydrocarbon receptor (AHR), REF receptor kinase, FKBP, Androgen Receptor (AR), Estrogen receptor (ER), Thyroid Hormone Receptor, HIV Protease, HIV Integrase, HCV Protease, and Acyl-protein Thioesterase-1 and-2 (APTI and APT2). A chemical compound can be selected from the group consisting of Heat Shock Protein 90 (HSP90) inhibitors, Kinase and Phosphatase inhibitors, MDM2 inhibitors, HDAC inhibitors, Human Lysine Methyltransferase Inhibitors, Angiogenesis inhibitors, and immunosuppressive compounds, which can bind to the substrate, of small molecules that can bind to proteins include, but are not limited to 4-hydroxytamoxifen (4-OHT), AC220, Afatinib, an aminopyrazole analog, an AR antagonist, BI-7273, Bosutinib, Ceritinib, Chloroalkane, Dasatinib, Foretinib, Gefitinib, a HIF-1α-derived (R)-hydroxyproline, HJB97, a hydroxyproline-based ligand, IACS-7e, Ibrutinib, an ibrutinib derivative, Jq1, Lapatinib, an LCL161 derivative, Lenalidomide, a nutlin small molecule, OTX015, a PDE4 inhibitor, Pomalidomide, a ripk2 inhibitor, RN486, Sirt2 inhibitor 3b, SNS-032, Steel factor, a TBK1 inhibitor, Thalidomide, a thalidomide derivative, a Thiazolidinedione-based ligand, a VH032 derivative, VHL ligand 2, VHL-1, VL-269, and derivatives thereof.

Non-limiting examples of small molecules that are conjugated to linear RNAs of the disclosure that bind to exemplary target proteins are provided in TABLE 4. Non-limiting examples of chemical compounds that are conjugated to linear RNAs of the disclosure that bind to exemplary substrate proteins are provided in TABLE 5.

TABLE 4

| | |
|---|---|
| Exemplary Chemical Compounds | Exemplary Target Protein(s) |
| An LCL161 derivative | IAP |
| VHL-1 | VHL |
| Pomalidomide | CRBN |
| Thalidomide | CRBN |
| Lenalidomide | CRBN |
| A thalidomide derivative | CRBN |
| A HIF-1α-derived (R)-hydroxyproline | VHL |
| VHL ligand 2 | VHL |
| VL-269 | VHL |
| VH 032 | VHL |
| A VH 032 derivative | VHL |
| A hydroxyproline-based ligand | VHL |

TABLE 5

| | |
|---|---|
| Exemplary Chemical Compounds | Exemplary Substrate Protein(s) |
| Dasatinib | BCR-Abl; c-ABL |
| Lapatinib | EGFR |
| Gefitinib | EGFR |
| Foretinib | c-met |
| Sirt2 inhibitor 3b | sirt2 |
| SNS-032 | CDK9 |
| AC220 | FLT3 |
| Ceritinib | ALK |
| Ibrutinib | BTK |
| 4-OHT | ERalpha |
| Jq1 | BRD2/3/4 |
| A PDE4 inhibitor | PDE4 |
| Chloroalkane | GFP-halotag7 |
| A Thiazolidinedione-based ligand | ERRalpha |
| A ripk2 inhibitor | RIPK2 |
| Bosutinib | c-ABL; BCR-ABL |
| OTX015 | BRD2/3/4 |
| Steel factor | FKBP12 |
| A TBK1 inhibitor | TBK1 |
| BI-7273 | BRD9 |
| Lapatinib | wild type EGFR; Exon 20 insertion EGFR; HER2 |
| Geftinib | Exon 19 deletion EGFR; L858R EGFR |
| Afatinib | EGFR |
| Foretinib | c-Met |
| Sirt 2 inhibitor 3b | Sirt2 |
| HJB97 | BRD2/3/4 |
| SNS-032 | CDK9 |
| An aminopyrazole analog | CDK9 |
| AC220 | FLT3 |
| RN486 | BTK |
| Ceritinib | ALK |
| An adrenergic receptor antagonist | Adrenergic receptors |
| An androgen receptor antagonist | Androgen receptors |
| IACS-7e | TRIM24 |
| An ibrutinib derivative | wild-type BTK; C481S BTK |
| A nutlin small molecule | MDM2 |

In some embodiments, a chemical compound binds to a target protein, wherein the target protein is an enzyme. The chemical compound can bind to post-translational modifying enzyme. The chemical compound can bind to a nitrosylase, an acetyltransferase, a deacetylase, a factor that modulates SUMOylation, a methyltransferase, a kinase, a phosphatase, a glycosyltransferase, a glycoside hydrolase, or a sulfotransferase. In some embodiments, the chemical compound binds to a factor that modulates, for example, acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, SUMOylation, succinylation, sulfation, S-sulfenylation, S-sulfinylation, succinylation, ubiquitination, uridylylation, or a combination thereof. For example, a chemical compound can bind to a ubiquitin ligase, thereby generating a complex. Examples of ligands that can bind to ubiquitin ligases include, but are not limited to, a HIF-1α-derived (R)-hydroxyproline, a hydroxyproline-based ligand, an LCL161 derivative, lenalidomide, pomalidomide, thalidomide, a thalidomide derivative, a VH032 derivative, VHL-1, VHL ligand 2, VL-269, and derivatives thereof.

In some embodiments, a chemical compound binds to a substrate protein, wherein the substrate protein is a disease associated protein. The chemical compound can bind to protein associated with cancer. The chemical compound can bind to a misfolded protein. For example, a chemical compound can bind to a substrate, thereby generating a complex.

In some embodiments, a linear RNA comprises a first conjugation moiety that is conjugated to a first chemical compound and a second conjugation moiety that is conjugated to a second chemical compound, wherein the first chemical compound binds to a target protein and the second chemical compound binds to a substrate protein of the target protein. In some embodiments, a linear RNA comprises a first conjugation moiety that is conjugated to a first chemical compound and a second conjugation moiety that is conjugated to a second chemical compound, wherein the first chemical compound is bound to a target protein and the second chemical compound is bound to a substrate protein of the target protein, thereby forming a complex.

In some embodiments, a linear RNA comprises a plurality of chemical compounds moieties. For example, the linear RNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90 or 100 or more chemical compounds or any number therein between. The linear RNA can comprise 1 chemical compound. The linear RNA can comprise 2 chemical compounds. The linear RNA can comprise 2 chemical compounds. The linear RNA can comprise 3 chemical compounds. The linear RNA can comprise 4 chemical compounds. The linear RNA can comprise 5 chemical compounds. The linear RNA can comprise 6 chemical compounds. The linear RNA can comprise 7 chemical compounds. The linear RNA can comprise 8 chemical compounds. The linear RNA can comprise 9 chemical compounds. The linear RNA can comprise 10 chemical compounds. The linear RNA can comprise 11 chemical compounds. The linear RNA can comprise 12 chemical compounds. The linear RNA can comprise 13 chemical compounds. The linear RNA can comprise 14 chemical compounds. The linear RNA can comprise 15 chemical compounds. The linear RNA can comprise 16 chemical compounds. The linear RNA can comprise 17 chemical compounds. The linear RNA can comprise 18 chemical compounds. The linear RNA can comprise 19 chemical compounds. The linear RNA can comprise 20 chemical compounds. The linear RNA can comprise 21 chemical compounds. The linear RNA can comprise 22 chemical compounds. The linear RNA can comprise 23 chemical compounds. The linear RNA can comprise 24 chemical compounds. The linear RNA can comprise 25 chemical compounds. The linear RNA can comprise 26 chemical compounds. The linear RNA can comprise 27 chemical compounds. The linear RNA can comprise 28 chemical compounds. The linear RNA can comprise 29 chemical compounds. The linear RNA can comprise 30 chemical compounds. The linear RNA can comprise 31 chemical compounds. The linear RNA can comprise 32 chemical compounds. The linear RNA can comprise 33 chemical compounds. The linear RNA can comprise 34 chemical compounds. The linear RNA can comprise 35 chemical compounds. The linear RNA can comprise 36 chemical compounds. The linear RNA can comprise 37 chemical compounds. The linear RNA can comprise 38 chemical compounds. The linear RNA can comprise 39 chemical compounds. The linear RNA can comprise 40 chemical compounds. The linear RNA can comprise 41 chemical compounds. The linear RNA can comprise 42 chemical compounds. The linear RNA can comprise 43 chemical compounds. The linear RNA can comprise 44 chemical compounds. The linear RNA can comprise 45 chemical compounds. The linear RNA can comprise 46 chemical compounds. The linear RNA can comprise 47 chemical compounds. The linear RNA can comprise 48 chemical compounds. The linear RNA can comprise 49 chemical compounds. The linear RNA can comprise 50 chemical compounds. The linear RNA can comprise 55 chemical compounds. The linear RNA can comprise 60 chemical compounds. The linear RNA can comprise 70 chemical compounds. The linear RNA can comprise 80 chemical compounds. The linear RNA can comprise 90 chemical compounds. The linear RNA can comprise 100 chemical compounds. In some embodiments, the plurality of chemical compounds are the same. In some embodiments, the plurality of chemical compounds are different. In some embodiments, a linear RNA comprises a first conjugation moiety and a second conjugation moiety. In some embodiments, a linear RNA comprises a first conjugation moiety that is conjugated to a first chemical compound and a second conjugation moiety that is conjugated to a second chemical compound, wherein the first chemical compound binds to a target and the second chemical compound binds to a substrate of the target.

### Conjugation to a Conjugation Moiety of a Linear Polyribonucleotide

A linear RNA of the disclosure can be conjugated, for example, to a chemical compound (e.g., a small molecule), an antibody or fragment thereof, a peptide, a protein, an aptamer, a drug, or a combination thereof. In some embodiments, a small molecule can be conjugated to a linear RNA, thereby generating a linear RNA comprising a small molecule. In some emodiments, two molecules are conjugated to a linear RNA. Such two molecules can be identical or different. In instances where a linear RNA is conjugated to two different molecules, e.g., a first chemical compound and a second chemical compound, such two different molecules can bind to biological molecules, e.g., molecules present in biological systems, such as proteins, nucleic acids, metabolites, etc. In some embodiments, a first chemical compound binds to a target molecule, and a second chemical compound can bind to a substrate molecule.

A linear RNA of the disclosure can comprise a conjugation moiety to facilitate conjugation to a chemical compound as described herein. A conjugation moiety is incorporated, for example, at a 5′ end, 3′ end, or internal site of a linear polyribonucleotide. A conjugation moiety can be incorporated chemically or enzymatically. For example, a conjugation moiety is incorporated during solid phase oligonuleotide synthesis, cotranscriptionally (e.g., with a tolerant RNA polymerase) or posttranscriptionally (e.g., with an RNA methyltransferase). A conjugation moiety can be a nucleotide analog, e.g., bromodeoxyuridine. A conjugation moiety can comprise a reactive or functional group, e.g., an azide group or an alkyne group. A conjugation moiety can be capable of undergoing a chemoselective reaction. A conjugation moiety can be capable of undergoing a biorthognal reaction. A conjugation moiety can be a hapten group, e.g., comprising digoxigenin, 2,4-dinitrophenyl, biotin, avidin, or are selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A conjugation moiety can comprise a diarylethene photoswitch capable of undergoing reversible electrocyclic rearrangement. A conjugation moiety can comprise a nucleophile, a carbanion, and/or an α,β-unsaturated carbonyl compound. In some instances, conjugation can include functional group modifications such as mesylate formation, sulfur alkylation, NHS ester formation, carbamate formation, carbonate formation, amide bond formation, or any combination thereof.

Thus, as described herein, a linear RNA can be conjugated to one or more molecules covalently or non-covalently, or a combination thereof.

In some embodiments, where a linear RNA is conjugated covalently, a linear RNA isconjugated via a chemical reaction, e.g., using click chemistry, Staudinger ligation, transition-metal catalyzed reactions, e.g., Pd-catalyzed C—C bond formation (e.g., Suzuki-Miyaura reaction), Michael addition, olefin metathesis, or inverse electron demand Diels-Alder. Click chemistry can utilize pairs of functional groups that rapidly and selectively react (“click”) with each other in appropriate reaction conditions. Non-limiting click chemistry reactions include azide-alkyne cycloaddition, copper-catalyzed 1,3-dipolar azide-alkyne cycloaddition (CuAAC), ruthenium-catalyzed azide-alkyne cycloaddition (RuAAC), strain-promoted Azide - Alkyne Click Chemistry reaction (SPAAC), tetrazine - alkene (e.g., trans-cyclooctene) ligation, or photo-click reactions (e.g., alkene-tetrazole photoreactions). Other types of conjugation chemistry can include Schiff-base formation, peptide ligation, isopeptide bond formation, etc.

Non-limiting examples of functionalized nucleotides include modified UTP analogs, modified ATP analogs, modified CTP analogs, and/or modified GTP analogs, and any combinations thereof. In some instances, functionalized nucleotides include azide and/or alkene functional groups. Examples of such modified nucleotides include azide modified UTP analogs, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-DBCO-PEG4-dCpG and 5-azidopropyl-UTP. In some embodiments, a circRNA comprises at least one 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 5-Azido-PEG4-CTP, 5-DBCO-PEG4-CTP, or 5-azidopropyl-UTP.

A single modified nucleotide of choice (e.g., modified A, C, G, U, or T containing an azide at the 2′-position) can be incorporated site-specifically under optimized conditions (e.g., via solid-phase chemical synthesis). A plurality of nucleotides containing an azide at the 2′-position can be incorporated, for example, by substituting a nucleotide during an in vitro transcription reaction (e.g., substituting UTP for 5-azido-C3-UTP).

A linear RNA conjugate can be generated using a copper-catalyzed click reaction, e.g., copper-catalyzed 1,3-dipolar azide-alkyne cycloaddition (CuAAC) of an alkyne-functionalized small molecule and an azide-functionalized polyribonucleic acid. A linear RNA can be conjugated with a small molecule. For example, a linear RNA can be modified at its 3′-end by a poly(A) polymerase with an azido-derivatized nucleotide. The azide can be conjugated to a small molecule via copper-catalyzed or strain-promoted azide-alkyne click reaction, and the linear RNA can be conjugated to another linear RNA or a circular polyribonucleotide.

A linear RNA conjugate can be generated using a Staudinger reaction. For example, a linear RNA comprising an azide-functionalized linear RNA can be conjugated with an alkyne-functionalized small molecule in the presence of triphenylphosphine-3,3’,3″-trisulfonic acid (TPPTS).

A linear RNA conjugate can be generated using a Suzuki-Miyaura reaction. For example, a linear RNA comprising a halogenated nucleotide analog can be subjected to Suzuki-Miyaura reaction in the presence of a cognate reactive partner. A linear RNA comprising 5-Iodouridine triphosphate (IUTP), for example, can be used in a catalytic system with Pd(OAc)₂ and 2-aminopyrimidine-4,6-diol (ADHP) or dimethylamino-substituted ADHP (DMADHP) to functionalize iodouridine-labeled linear RNA in the presence of various boronic acid and ester substrates. In another example, a linear RNA comprising 8-bromoguanosine can be reacted with arylboronic acids in the presence of a catalytic system made of Pd(OAc)₂ and a water-soluble triphenylphosphan-3,3’,3″-trisulfonate ligand.

A linear RNA conjugate can be generated using Michael addition, for example, via reaction of an an electron-rich Michael Donor with an α,β-unsaturated compound (Michael Acceptor).

### Target

A chemical compound conjugated to conjugation moiety of a linear RNA can bind to a target. A binding site (e.g., an aptmer) of a linear RNA can bind to target. Targets include, but are not limited to, nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof. A target can be a target protein.

In some embodiments, a target is a polypeptide, a protein, or any fragment thereof. For example, a target is a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a tyrosine kinase, a serine/threonine kinase, a phosphatase, an aromatase, a phosphodiesterase, a cyclase, a helicase, a protease, an oxidoreductase, a reductase, a transferase, a hydrolase, a lyase, an isomerase, a glycosylase, a extracellular matrix protein, a ligase, a ubiquitin ligase, an ion transporter, a channel, a pore, an apoptotic protein, a cell adhesion protein, a pathogenic protein, an aberrantly expressed protein, an transcription factor, a transcription regulator, a translation protein, an epigenetic factor, an epigenetic regulator, a chromatin regulator, a chaperone, a secreted protein, a ligand, a hormone, a cytokine, a chemokine, a nuclear protein, a receptor, a transmembrane receptor, a receptor tyrosine kinase, a G-protein coupled receptor, a growth factor receptor, a nuclear receptor, a hormone receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, a proto-oncogene, an oncogene, a tumor-suppressor gene, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, mammalian, or insect cells (e.g., proteins overexpressed by the organisms). In some embodiments, a target is a polypeptide with a mutation, insertion, deletion, or polymorphism. In some embodiments, a target is a polypeptide naturally expressed by a cell (e.g., a healthy cell or a cell associated with a disease or condition). In some embodiments, a target is an antigen, such as a polypeptide used to immunize an organism or to generate an immune response in an organism, such as for antibody production.

A target protein can comprise an enzyme that modulates a substrate, e.g., a substrate protein. In some embodiments, a target protein modulates a substrate protein by post-translational modification, for example, acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, SUMOylation, succinylation, sulfation, S-sulfenylation, S-sulfinylation, ubiquitination, uridylylation, or a combination thereof. Examples of substrate proteins include, but are not limited to, an adrenergic receptor, ALK, an androgen receptor, BCR-ABL, BRD2, BRD3, BRD4, BRD9, BTK, c-ABL, c-Met, CDK9, EGFR, ERalpha, ERRalpha, FLT3, FKBP12, GFP-halotag7, HER2, MDM2, p53, PDE4, RIPK2, sirt2, TBK1, TRIM24, or a combination thereof.

In some embodiments, a target protein is a ubiquitin ligase, an E3 ubiquitin ligase, a HECT ubiquitin ligase, a RING-finger ubiquitin ligase, a U-box ubiquitin ligase, a PHD-finger ubiquitin ligase, or a combination thereof. In some embodiments, a target protein is a ubiquitin ligase adaptor protein/complex, a proteasome adaptor protein/complex, or a proteasome protein/complex, such as RNP1, RPN10, RPN13, p62, Rad23/HR23, Dsk2/PLIC/Ubiquilin, and Ddi1. In some embodiments, a target protein is a ubiquitin adaptor that can direct substrates to autophagic vacuoles, such as p62/SQSTM-⅟Sequestosome-1, neighbor of BRCA1 gene 1 (NBR1), HDAC6, ESCRT-0 complex, ESCRT-I complex, ESCRT-II complex, and ESCRT-III complex. In some embodiments, a target can be a molecule that directs a substrate protein to a lysosome by endocytosis (e.g., an endocytic receptor), a molecule that directs a substrate protein to a lysosome by phagocytosis (e.g., a phagocytic receptor), a molecule that directs a substrate protein to a lysosome via autophagy, a molecule that directs a substrate protein to a lysosome via macroautophagy, a molecule that directs a substrate protein to a lysosome via microautophagy, a molecule that directs a substrate protein to a lysosome via chaperone-mediated autophagy, a molecule that directs a substrate protein to a lysosome via a multivesicular body pathway.

Further examples of target proteins include, but are not limited to, AFF4, AMFR, ANAPC11, ANKIBI, APC/C, AREL1, AR1H1, ARIH2, BARD1, beta-TrCP1, BFAR, BIRC2, BIRC3, BIRC7, BIRC8, BMII, BRAP, BRCA1, c..IAP1CBL, CBLB, CBLC, CBLL1, CCDC36, CCNB11P1, Cereblon (CRBN), CCFRRF1, CHFR, CHIP, CNOT4, CUL9, CYHR1, DCST1, DTX1, DTX2, DTX3, DTX3L, DTX4, DZIP3, E4F1, E6AP, FANCL, G2E3, gp78, HACE1, HECTD1, HECTD2, HECTD3, HECTD4, HECW1, HECW2, HERC1, HERC2, HERC3, HERC4, HERC5, HERC6, HLTF, HOIL-IL, HOIP, HUL5, HUWE1, IAP, IRF2BP1, IRF2BP2, IRF2BPL, Itch, KCMF1, KMT2C, KMT2D, LNX1, LNX2, LONRF1, LONRF2, LONRF3, LRSAM1, LTN1, LUBAC, MAEA, MAP3K1, MARCH1, MARCH10, MARCH11, MARCH2, MARCH3, MARCH4, MARCH5, MARCH6, MARCH7, MARCH8, MARCH9, Mdm2, MDM4, MECOM, MEX3A, MEX3B, MEX3C, MEX3D, MGRN1, MIB1, MIB2, MID1, MID2, MKRN1, MKRN2, MKRN3, MKRN4P, MNAT1, MSL2, MUL1, MYCBP2, MYLIP, NEDD4, NEDD4L, NEURL1, NEURL1B, NEURL3, NFX1, NFXL1, NHLRC1, NOSIP, NSMCE1, Parkin, PARK2, PCGF1, PCGF2, PCGF3, PCGF5, PCGF6, PDZRN3, PDZRN4, PELI1, PELI2, PELI3, PEX10, PEX12, PEX2, PHF7, PHRF1, PJA1, PJA2, PLAG1, PLAGL1, PML, PPIL2, PRPF19, pVHL, RAD18, RAG1, RAPSN, RBBP6, RBCK1, RBX1, RC3H1, RC3H2, RCHY1, RFFL, RFPL1, RFPL2, RFPL3, RFPL4A, RFPL4AL1, RFPL4B, RFWD2, RFWD3, RING1, RLF, RLIM, RMND5A, RMND5B, RNF10, RNF103, RNF11, RNFI. 11, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, Rsp5, RSPRY1, San1, SCAF11, SCF, SHARPIN, SH3RF1, SH3RF2, SH3RF3, SHPRH, SIAH1, SIAH2, SIAH3, SMURF1, SMURF2, STUB1, SYVN1, TMEM129, Topors, TRAF2, TRAF3, TRAF4, TRAF5, TRAF6, TRAF7, TRAIP, TRIM10, TRIM11, TRIM13, TRIM15, TRIM17, TRIM2, TRIM21, TRIM22, TRIM23, TRIM24, TRIM25, TRIM26, TRIM27, TRIM28, TRIM3, TRIM31, TRIM32, TRIM33, TRIM34, TRIM35, TRIM36, TRIM37, TRIM38, TRIM39, TRIM4, TRIM40, TRIM41, TRIM42, TRIM43, TRIM43B, TRIM45, TRIM46, TRIM47, TRIM48, TRIM49, TRIM49B, TRIM49C, TRIM49D1, TRIM5, TRIM50, TRIM51, TRIM52, TRIM 54., TRIM55, TRIM56, TRIM58, TRIM59, TRIM6, TRIM60, TRIM61, TRIM62, TRIM63, TRIM64, TRIM64B, TRIM64C, TRIM65, TRIM67, TRIM68, TRIM69, TRIM7, TRIM71, TRIM72, TRIM73, TRIM74, TRIM75P, TRIM77, TRIM8, TRIM9, TRIML1, TRIML2, TRIP12, TTC3, UBE3A, UBE3B, UBE3C, UBE3D, UBE4A, UBE4B, UBOX5, UBR1, UBR2, UBR3, UBR4, UBR5, UBR7, UHRF1, UHRF2, UNK, UNKL, VHL, VPS11, VPS18, VPS41, VPS8, WDR59, WDSUB1, WWP1, WWP2, XIAP, ZBTB12, ZFP91, ZFPL1, ZNF280A, ZNF341, ZNF511, ZNF521, ZNF598, ZNF645, ZNRF1, ZNRF2, ZNRF3, ZNRF4, Zswim2, and ZXDC. For example, a target protein is selected from the group consisting of von Rippel-Lindau (VHL); cereblon; XIAP; E3A; MDM2; Anaphase-promoting complex (APC); UBR5 (EDDI); SOCS/ BC-box/ eloBC/ CUL5/ RING; LNXp80; CBX4; CBLLI; HACEI; HECTDI; HECTD2; HECTD3; HECWI; HECW2; HERCI; HERC2; HERC3; HERC4; HUWEI; ITCH; NEDD4; NEDD4L; PPIL2; PRPF19; PIASI; PIAS2; PIAS3; PIAS4; RANBP2; RNF4; RBXI; SMURFI; SMURF2; STUBI; TOPORS; TRIP12; UBE3A; UBE3B; UBE3C; UBE4A; UBE4B; UBOX5; UBR5; WWPI; WWP2; Parkin; A20/TNFAIP3; AMFR/gp78; ARA54; beta-TrCPI/BTRC; BRCAI; CBL; CHIP/STUB I; E6; E6AP/UBE3A; F-box protein 15IFBXOIS; FBXW7/Cdc4; GRAIL/RNF128; HOIP/RNF3 1; cIAP-⅟HIAP-2; cIAP- 2/HIAP-1; cIAP (pan); ITCH/AIP4; KAPI; MARCH8;; Mind Bomb ⅟MIBI; Mind Bomb 2/MIB2; MuRFI/TRIM63; NDFIPI; NEDD4; NleL; Parkin; RNF2; RNF4; RNF8; RNF168; RNF43; SARTI; Skp2; SMURF2; TRAF-1; TRAF-2; TRAF-3; TRAF-4; TRAF-5; TRAF-6; TRIMS; TRIM21; TRIM32; UBR5; and ZNRF3

Further examples of target proteins include, but are not limited to, E3 ligases from Tables 13-27 in EP3458101, which is hereby incorporated by reference in its entirety.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2} and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341 :544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments can include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, trispecific, tetraspecific, pentaspecific, hexaspecific, heptaspecific, or octaspecific antibodies are generated, e.g., by recombinantly joining a combination of any two or more antigen binding agents (e.g., Fab, F(ab)₂, scFv, Fv, IgG). Multi-specific antibodies can be used to bring two or more targets into close proximity, e.g., degradation machinery and a target substrate to degrade, or a ubiquitin ligase and a substrate to ubiquitinate. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is entirely fortuitous and plays no role whatsoever in the binding of an aptamer to its cognate target. Aptamers must also be differentiated from the naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand are short, isolated, non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences will differ from aptamers as a result of the relatively low binding affinity used in nature as compared to tightly binding aptamers. Aptamers are capable of specifically binding to selected partners and modulating the partner’s activity or binding interactions, e.g., through binding, aptamers may block their partner’s ability to function. The functional property of specific binding to a partner is an inherent property an aptamer. A typical aptamer is 6-35 kDa in size (20-100 nucleotides), binds its partner with micromolar to sub-nanomolar affinity, and may discriminate against closely related targets (e.g., aptamers may selectively bind related proteins from the same gene family). In some embodiments, an aptamer is from 250-500 nucleotides. Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides.

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from -0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a target is a cell. For example, a target is an intact cell, a cell treated with a compound (e.g., a drug), a fixed cell, a lysed cell, or any combination thereof. In some embodiments, a target is a single cell. In some embodiments, a target is a plurality of cells.

### Substrate

A target can modulate a substrate. A chemical compound conjugated to a conjugation moiety of a linear RNA can bind to a substrate. A binding site in a linear RNA can be bind a substrate. Substrates include, but are not limited to, nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al., (2004) PNAS, 101:8420-24). For example, a substrate can be a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof. A substrate can be a substrate protein. The substrate protein can be modified by a target protein, which can modulate a cellular process involving the substrate protein.

A substrate protein can be a single protein. A substrate protein can be a protein aggregate. In some embodiments, a substrate protein is a protein, organelle, lipoprotein, glycoprotein, phosphoprotein, hemoprotein, flavoprotein, metalloprotein, ribonucleoprotein, or any combination thereof. A substrate protein can be associated with a disease or condition. For example, a substrate protein is a disease-associated protein. In some embodiments, a substrate protein is a misfolded protein. In some embodiments, a substrate protein comprises a mutation as compared to a wild-type version of the substrate protein. Substrate proteins include, but are not limited to, adrenergic receptors, ALK, androgen receptors, BCR-ABL, BRD2, BRD3, BRD4, BRD9, BTK, c-ABL, c-Met, CDK9, EGFR, ERalpha, ERRalpha, FLT3, FKBP12, GFP-halotag7, HER2, MDM2, p53, PDE4, RIPK2, sirt2, TBK1, TRIM24, and combinations thereof. A substrate protein can be selected from the group consisting of FoxOl, HDAC, DP-1, E2F, ABL, ALK, AMPK, BRK, BRSK I, BRSK2, BTK, CAMKKI, CAMKK alpha, CAMKK beta, Rb, Suv39HI, SCF, pl9INK4D, GSK-3, pi 8 INK4, myc, cyclin E, CDK2, CDK9, CDG4/6, Cycline D, pl6 INK4A, cdc25A, BMII, SCF, Akt, CHKl/2, CI delta, CKI gamma, C 2, CLK2, CSK, DDR2, DYRKIA/2/3, EF2K, EPH-A2/A4/Bl/B2/B3/B4, EIF2A 3, Smad2, Smad3, Smad4, Smad7, p53, p21 Cipl, PAX, Fyn, CAS, C3G, SOS, Tal, Raptor, RACK-I, CRK, Rapl, Rae, KRas, NRas, HRas, GRB2, FAK, PBK, spred, Spry, mTOR, MPK, LKBl, PAK 1/2/4/5/6, PDGFRA, PYK.2, Src, SRPKI, PLC, PKC, PKA, PKB, alpha/beta, PKC alpha/gamma/zeta, PKD, PLKl, PRAK, PRK2, RIPK2, WA VE-2, TSC2, DAPKl, BAD, IMP, C-TAKI, TAKI, TAOl, TBKI, TESKI, TGFBRI, TIE2, TLKI, TrkA, TSSKI, TTBKI/2, TTK, Tpl2/cotl, MEKI, MEK2, PLDL Erkl, Erk2, Erk5, Erk8, p90RSK, PEA- 15, SRF, p27 KIPI, TIF 1a, HMGNI, ER81, MKP-3, c-Fos, FGF-Rl, GCK, GSK3 beta, HER4, HIPKI/2/3/, IGF-IR, cdc25, UBF, LAMTOR2, Statl, StaO, CREB, JAK, Src, SNCA, PTEN, NF- kappaB, HECTH9, Bax, HSP70, HSP90, Apaf-1, Cyto c, BCL-2, Bcl-xL, BCL-6, Smac, XIAP, Caspase-9, Caspase-3, Caspase-6, Caspase-7, CDC37, TAB, IKK, TRADD, TRAF2, RIPI, FLIP, TAKI, JNKl/2/3, Lek, A-Raf, B-Raf, C-Raf, MOS, MLKl/3, MN 1/2, MSKl, MST2/3/4, MPSKI, MEKKl, ME K4, MEL, ASKI, MINK I, MKK l /2/3/4/6/7, NE, 2a/6/7, NUAKI, OSRI, SAP, STK33, Syk, Lyn, PDKI, PHK, PIM 1/2/3, Ataxin- 1, mTORCl, MDM2, p21 Wafl, Cyclin Dl, Lamln A, Tp12, Myc, catenin, Wnt, IKK-beta, IKKgamma, IKK-alpha, IKK-epsilon, ELK, p65Re1A, IRAKI, IRA 2, IRAK4, IRR, FADD, TRAF6, TRAF3, MKK3, MKK6, ROCK2, RSKI/2, SGK 1, SmMLCK, SIK2/3, ULKI/2, VEGFRI, WNK 1, YESI, ZAP70, MAP4K3, MAP4K5, MAPKlb, MAPKAP-K2 K3, p38, alpha/beta/delta/gamma MAPK, Aurora A, Aurora B, Aurora C, MCAK, Clip, MAPKAPK, FAK, MARK l /2/3/4, Mucl, SHC, CXCR4, Gap-I, Myc, beta-catenin/TCF, Cbl, BRM, Mell, BRD2, BRD3, BRD4, AR, RAS, ErbB3, EGFR, IREI, HPKI, RIPK2, and ERa, PCAF/GCN5, including all variants, mutations, splice variants, indels and fusions thereof.

Substrate proteins is modified by a post-translational modification of a peptide sequence, e.g., acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, succinylation, sulfation, S-sulfenylation, S-sulfinylation, succinylation, sumoylation, ubiquitination, uridylylation, or a combination thereof.

For example, a substrate protein can be marked for degradation via ubiquitination. Substrate proteins can be marked for degradation by the attachment of ubiquitin to the amino group of the side chain of a lysine residue. Additional ubiquitins can then be added to form a polyubiquitin chain. Such polyubiquinated proteins can then be directed to, for example, a proteasome, autophagosome, or lysosome for degradation.

In some embodiments, a substrate is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2} and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341 :544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, trispecific, tetraspecific, pentaspecific, hexaspecific, heptaspecific, or octaspecific antibodies can be generated, e.g., by recombinantly joining a combination of any two or more antigen binding agents (e.g., Fab, F(ab)₂, scFv, Fv, IgG). Multi-specific antibodies can be used to bring two or more targets into close proximity. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a substrate is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a substrate is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a substrate is a heterologous polynucleotide. For example, a substrate is a polynucleotide produced from bacterial (e.g., E. coli), yeast, mammalian, or insect cells (e.g., polynucleotides heterologous to the organisms). In some embodiments, a substrate is a polynucleotide with a mutation, insertion, deletion, or polymorphism.

In some embodiments, substrate is an aptamer. As described herein, an aptamer is an isolated nucleic acid molecule that binds with high specificity and affinity to a binding moiety, such as a protein. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides.

In some embodiments, a substrate is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from -0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a substrate is a cell. For example, a substrate is an intact cell, a cell treated with a compound (e.g., a drug), a fixed cell, a lysed cell, or any combination thereof. In some embodiments, a substrate is a single cell. In some embodiments, a target is a plurality of cells.

### Modulation of a Substrate

A linear RNA as disclosed herein can modulate a cellular process by modifying a substrate. In some embodiments, a linear RNA includes comprises a conjugation moiety for binding to chemical compound. The conjugation moiety can be a modified polyribonucleotide. The chemical compound can be conjugated to the linear RNA by the conjugation moiety. In some embodiments, the chemical compound binds to a target and mediates modulation of a substrate of the target. In some embodiments, a first chemical compound binds to a target and a second chemical copound binds to a substrate and the target mediates modulation of a substrate. In some embodiments, a linear RNA binds a substrate of a target and a chemical compound conjugated to the linear RNA by the conjugation moiety binds the target to bring together the target and its substrate to mediate modification of the substrate, e.g., post-translational modification. In some embodiments, a linear RNA binds a substrate of a target and a chemical compound conjugated to the linear RNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In some embodiments, the linear polyribonucleotide as disclosed herein persists in a cell or subject. In some embodiments, the linear polyribonucleotide as disclosed herein persists in a cell or subject longer than a small molecule. In some embodiments, the linear polyribonucleotide as disclosed herein persists in a cell or subject longer than a corresponding proteolysis targeting chimera small molecule.

Modulation of a substrate protein comprises, for example, chemical modification of a substrate protein. In some embodiments, modulation of a substrate protein comprises post-translational modification of a peptide sequence, e.g., acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, succinylation, sulfation, S-sulfenylation, S-sulfinylation, succinylation, sumoylation, ubiquitination, uridylylation, or a combination thereof.

Modulation of a substrate protein can alter the biological activity of the substrate protein. In some embodiments, modulation of a substrate protein promotes or inhibits interaction of two or more molecules (e.g., proteins), promotes or inhibits formation of a complex (e.g., a protein complex), or promotes or inhibits of an enzymatic reaction. In some embodiments, modulation of a substrate protein alters the stability of a molecule (e.g., the substrate protein), or promotes or inhibits synthesis of a molecule (e.g., promotes or inhibits transcription, translation, or enzymatic processing). In some embodiments, modulation of a substrate protein promotes or inhibits of ubiquitination, e.g., ubiquitination of one of the one or more proteins. In some embodiments, modulation of a substrate protein promotes or inhibits degradation of a protein, e.g., degradation of one or more target proteins via proteasomal degradation or lysosomal degradation. In some embodiments, modulation of a substrate protein promotes or inhibits a signal transduction pathway, results in a conformational change, (e.g., a conformational change of a substrate protein protein), results in increased or decreased biological activity of the substrate protein, or alters localization of the substrate protein (e.g., alters sub-cellular localization). In some embodiments, modulation of a substrate protein alters a disease or condition, e.g., reduces a disease or condition in a subject. In some embodiments, modulation of a substrate protein promotes or inhibits DNA damage repair (e.g., increases or decreases the accuracy of DNA damage repair, or increases or decreases the processive efficiency of DNA damage repair). In some embodiments, modulation of a substrate protein promotes or inhibits cell cycle progression, promotes or inhibits cell division (e.g., inhibits cell division of a disease-associated cell subset), promotes or inhibits apoptosis (e.g., apoptosis of a disease-associated cell subset). In some embodiments, modulation of a substrate protein promotes or inhibits epigenetic modifications (e.g., DNA methylation or histone modification). In some embodiments, modulation of a substrate protein promotes or inhibits gene expression by promoting or inhibiting epigenetic modifications.

In some embodiments, a linear RNA described herein is used to promote degradation of one or more substrate proteins. A linear RNA of the disclosure can be used, for example, to direct one or more substrate proteins to degradation machinery, to bring one or more substrate proteins in close proximity with degradation machinery, to bring one or more substrate proteins in close proximity with an enzyme that can mark the substrate protein for degradation, to reduce stability of a substrate protein (e.g., shorten the substrate protein half-life), to promote association of a substrate protein with an adaptor protein that is involved in a degradation process, to promote association of a substrate protein with a sorting agent that can sort the substrate protein into a degradative pathway, or a combination thereof. Substrate proteins can be degraded, for example, by a proteasomal pathway, a lysosomal pathway, an autophagic pathway, or a combination thereof.

Ubiquitination can be a multi-step reaction which involves subsequent action of three types of enzymes: E1 ubiquitin-activating, E2 ubiquitin-conjugating enzymes and E3 ubiquitin-ligases. Ubiquitin can be bound to a substrate protein as a monomer at a single (monoubiquitination) or multiple (multi-monoubiquitination) Lys residues. A ubiquitin moiety can be further polymerized by additional ubiquitins (polyubiquitination) via any of its seven Lys residues (Lys6, Lys11, Lys27, Lys29, Lys33, Lys48, and Lys63), or the N-terminal methionine (Met1). A single ubiquitin polymer can comprise one type of ubiquitin linkages (homotypic), or mixed ubiquitin linkages (heterotypic), in which a ubiquitin is joined to other ubiquitins through two or more different linkages. In some cases, ubiquitin is also modified at two or more sites, forming a branched polymer. Ubiquitin on substrate proteins can be modified by ubiquitin-like modifiers such as SUMO, NEDD8, and ISG15, or small molecule chemicals such as phosphate and acetate. The ubiquitin linkages and their modifications can generate distinct structures and recruit specific downstream effectors. Ubiquitin chains can bind to adaptors which can decode the distinct structures of ubiquitin chains and transfer the information on the substrate proteins to downstream machinery.

Ubiquitinated substrate proteins can be degraded by a proteasome. For example, adaptors (e.g., RNP1, RPN10, RPN13, p62, Rad23/HR23, Dsk2/PLIC/Ubiquilin, Ddi1) can deliver ubiquitinated substrate proteins to the proteasome. The substrate protein can then be deubiquitinated and threaded into the interior of the proteasome, where it can be degraded by chymotrypsin-, trypsin-, and caspase-like proteolytic activities.

Ubiquitinated substrate proteins can also be delivered to autophagosomes and/or lysosomes for degradation. For example, some ubiquitin adaptors link the substrate proteins to autophagic vacuoles (e.g., p62/SQSTM-⅟Sequestosome-1, neighbor of BRCA1 gene 1 (NBR1), HDAC6, ESCRT-0 complex, ESCRT-I complex, ESCRT-II complex, ESCRT-III complex). These adaptors can direct ubiquitinated substrate proteins to autophagic vacuoles, e.g., by binding ubiquitin on substrate proteins using ubiquitin binding domains and LC3 on autophagic vacuoles using LIR domains.

In some embodiments, ubiquitinated substrate proteins are not degraded by the proteasome but are modulated in other ways. The diversity of possible effects of ubiquitination can be related to the number of ubiquitin modifications present on a substrate protein (e.g., monoubiquitination or multi-monoubiquitination), characteristics of polyubiquitin chains, e.g., linear versus branched, type of linkages present (e.g., Lys6, Lys11, Lys27, Lys29, Lys33, Lys48, Lys63, Met1), homotypic versus heterotypic chains, modifications by ubiquitin-like modifiers such as SUMO, NEDD8, and ISG15, or small molecule chemicals such as phosphate and acetate, and downstream effectors, e.g., ubiquitin adaptors which can decode the distinct structures of ubiquitin chains.

Ubiquitination of a substrate protein can affect, for example, cell cycle regulation, DNA damage responses, substrate trafficking (e.g., trafficking of proteins to or from the plasma membrane), endocytosis, innate immunity, and intracellular signaling. For example, ubiquitination of a substrate protein can increase or decrease biological activity, increase or decrease interaction with a partner, or increase or decrease activation of a signal transduction pathway. Ubiquitination of substrate proteins can have effects that include but are not limited to modulation of immune and inflammatory signaling processes (e.g., modulation of NF-κB transcription factor activation, modulation of T and B cell development, modulation of cytokine signaling, modulation of TNF signaling pathways, modulation of NOD-like receptor signaling, modulation of TLR signaling, modulation of IL-1B signaling, modulation of RIG-I-like receptor signaling), modulation of cell death, modulation of embryonic development, modulation of autoimmune disease, modulation of JNK phosphorylation, modulation of Wnt signaling, and combinations thereof.

In some embodiments, a linear RNA described herein is used to promote ubiquitination of a substrate protein (e.g., for proteasomal and/or lysosomal degradation). In some embodiments, a linear RNA described herein is used to promote ubiquitination of a substrate protein without further administerin a ubiquitin ligase (e.g., the linear RNA associated degradation uses endogenous ubiquitin ligase). In some embodiments, linear RNA comprises a first binding site that binds a substrate protein, and a second binding site that binds a ubiquitin ligase. In some embodiments, a linear RNA comprises a first binding site that binds a substrate protein, and a conjugation moiety bound to a small molecule that binds a ubiquitin ligase. In some embodiments, a linear RNA comprises a conjugation moiety bound to a small molecule that binds a substrate protein, and a binding site that binds a ubiquitin ligase. A linear RNA of the disclosure binds, for example, an E3 ubiquitin ligase, a HECT ubiquitin ligase, a RING-finger ubiquitin ligase, a U-box ubiquitin ligase, a PHD-finger ubiquitin ligase, or a combination thereof. For example, a linear RNA of the disclosure can bind one or more ubiquitin ligases including, but not limited to, AFF4, AMFR, ANAPC11, ANKIB1, APC/C, AREL1, ARIH1, ARIH2, BARD1, beta-TrCP1, BFAR, BIRC2, BIRC3, BIRC7, BIRC8, BMI1, BRAP, BRCA1, c-IAP1CBL, CBLB, CBLC, CBLL1, CCDC36, CCNB1IP1, Cereblon (CRBN), CGRRF1, CHFR, CHIP, CNOT4, CUL9, CYHR1, DCST1, DTX1, DTX2, DTX3, DTX3L, DTX4, DZIP3, E4F1, E6AP, FANCL, G2E3, gp78, HACE1, HECTD1, HECTD2, HECTD3, HECTD4, HECW1, HECW2, HERC1, HERC2, HERC3, HERC4, HERC5, HERC6, HLTF, HOIL-IL, HOIP, HUL5, HUWE1, IAP, IRF2BP1, IRF2BP2, IRF2BPL, Itch, KCMF1, KMT2C, KMT2D, LNX1, LNX2, LONRF1, LONRF2, LONRF3, LRSAM1, LTN1, LUBAC, MAEA, MAP3K1, MARCH1, MARCH10, MARCH11, MARCH2, MARCH3, MARCH4, MARCH5, MARCH6, MARCH7, MARCH8, MARCH9, Mdm2, MDM4, MECOM, MEX3A, MEX3B, MEX3C, MEX3D, MGRN1, MIB1, MIB2, MID1, MID2, MKRN1, MKRN2, MKRN3, MKRN4P, MNAT1, MSL2, MUL1, MYCBP2, MYLIP, NEDD4, NEDD4L, NEURL1, NEURL1B, NEURL3, NFX1, NFXL1, NHLRC1, NOSIP, NSMCE1, Parkin, PARK2, PCGF1, PCGF2, PCGF3, PCGF5, PCGF6, PDZRN3, PDZRN4, PELI1, PELI2, PELI3, PEX10, PEX12, PEX2, PHF7, PHRF1, PJA1, PJA2, PLAG1, PLAGL1, PML, PPIL2, PRPF19, pVHL, RAD18, RAG1, RAPSN, RBBP6, RBCK1, RBX1, RC3H1, RC3H2, RCHY1, RFFL, RFPL1, RFPL2, RFPL3, RFPL4A, RFPL4AL1, RFPL4B, RFWD2, RFWD3, RING1, RLF, RLIM, RMND5A, RMND5B, RNF10, RNF103, RNF11, RNF111, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, Rsp5, RSPRY1, San1, SCAF11, SCF, SHARPIN, SH3RF1, SH3RF2, SH3RF3, SHPRH, SIAH1, SIAH2, SIAH3, SMURF1, SMURF2, STUB1, SYVN1, TMEM129, Topors, TRAF2, TRAF3, TRAF4, TRAF5, TRAF6, TRAF7, TRAIP, TRIM10, TRIM11, TRIM13, TRIM15, TRIM17, TRIM2, TRIM21, TRIM22, TRIM23, TRIM24, TRIM25, TRIM26, TRIM27, TRIM28, TRIM3, TRIM31, TRIM32, TRIM33, TRIM34, TRIM35, TRIM36, TRIM37, TRIM38, TRIM39, TRIM4, TRIM40, TRIM41, TRIM42, TRIM43, TRIM43B, TRIM45, TRIM46, TRIM47, TRIM48, TRIM49, TRIM49B, TRIM49C, TRIM49D1, TRIM5, TRIM50, TRIM51, TRIM52, TRIM54, TRIM55, TRIM56, TRIM58, TRIM59, TRIM6, TRIM60, TRIM61, TRIM62, TRIM63, TRIM64, TRIM64B, TRIM64C, TRIM65, TRIM67, TRIM68, TRIM69, TRIM7, TRIM71, TRIM72, TRIM73, TRIM74, TRIM75P, TRIM77, TRIM8, TRIM9, TRIML1, TRIML2, TRIP12, TTC3, UBE3A, UBE3B, UBE3C, UBE3D, UBE4A, UBE4B, UBOX5, UBR1, UBR2, UBR3, UBR4, UBR5, UBR7, UHRF1, UHRF2, UNK, UNKL, VHL, VPS11, VPS18, VPS41, VPS8, WDR59, WDSUB1, WWP1, WWP2, XIAP, ZBTB12, ZFP91, ZFPL1, ZNF280A, ZNF341, ZNF511, ZNF521, ZNF598, ZNF645, ZNRF1, ZNRF2, ZNRF3, ZNRF4, Zswim2, ZXDC, proteins coded for by the aforementioned genes, or combinations thereof. For example, a linear RNA of the disclosure binds one or more ubiquitin ligases selected from the group consisting of von Rippel-Lindau (VHL); cereblon; XIAP; E3A; MDM2; Anaphase-promoting complex (APC); UBR5 (EDDI); SOCS/ BC-box/ eloBC/ CUL5/ RING; LNXp80; CBX4; CBLLI; HACEI; HECTDI; HECTD2; HECTD3; HECWI; HECW2; HERCI; HERC2; HERC3; HERC4; HUWEI; ITCH; NEDD4; NEDD4L; PPIL2; PRPF19; PIASI; PIAS2; PIAS3; PIAS4; RANBP2; RNF4; RBXI; SMURFI; SMURF2; STUBI; TOPORS; TRIP12; UBE3A; UBE3B; UBE3C; UBE4A; UBE4B; UBOX5; UBR5; WWPI; WWP2; Parkin; A20/TNFAIP3; AMFR/gp78; ARA54; beta-TrCPl/BTRC; BRCAI; CBL; CHIP/STUB I; E6; E6AP/UBE3A; F-box protein 15/FBXOIS; FBXW7/Cdc4; GRAIL/RNF128; HOIP/RNF3 l; cIAP-1/HIAP-2; cIAP- 2/HIAP-l; cIAP (pan); ITCH/AIP4; KAPI; MARCH8;; Mind Bomb 1/MIBI; Mind Bomb 2/MIB2; MuRFl/TRIM63; NDFIPI; NEDD4; NleL; Parkin; RNF2; RNF4; RNF8; RNF168; RNF43; SARTI; Skp2; SMURF2; TRAF-1; TRAF-2; TRAF-3; TRAF-4; TRAF-5; TRAF-6; TRIMS; TRIM21; TRIM32; UBR5; and ZNRF3. A circRNA of the disclosure binds one or more ubiquitin ligases including, but not limited to, E3 ligases from Tables 13-27 in EP3458101, which is hereby incorporated by reference in its entirety.

In some embodiments, a linear RNA described herein can be used to direct a substrate protein to proteasomal degradation without binding an E3 ubiquitin ligase. For example, a linear RNA can comprise a first binding site that binds a substrate protein, and a second binding site that directs the substrate protein to a proteasome (e.g., via binding a ubiquitin ligase adaptor protein/complex, a proteasome adaptor protein/complex, or a proteasome protein/complex). A linear RNA of the disclosure can bind, for example, RNP1, RPN10, RPN13, p62, Rad23/HR23, Dsk2/PLIC/Ubiquilin, Ddi1, or a combination thereof. A linear RNA of the disclosure binds, for example, FoxOl, HDAC, DP-1, E2F, ABL, ALK, AMPK, BRK, BRSK I, BRSK2, BTK, CAMKKI, CAMKK alpha, CAMKK beta, Rb, Suv39HI, SCF, pl9INK4D, GSK-3, pi 8 INK4, myc, cyclin E, CDK2, CDK9, CDG4/6, Cycline D, pl6 INK4A, cdc25A, BMII, SCF, Akt, CHKl/2, CI delta, CKI gamma, C 2, CLK2, CSK, DDR2, DYRKIA/2/3, EF2K, EPH-A2/A4/B1/B2/B3/B4, EIF2A 3, Smad2, Smad3, Smad4, Smad7, p53, p21 Cipl, PAX, Fyn, CAS, C3G, SOS, Tal, Raptor, RACK-I, CRK, Rapl, Rae, KRas, NRas, HRas, GRB2, FAK, PBK, spred, Spry, mTOR, MPK, LKBl, PAK 1/2/4/5/6, PDGFRA, PYK.2, Src, SRPKI, PLC, PKC, PKA, PKB, alpha/beta, PKC alpha/gamma/zeta, PKD, PLKl, PRAK, PRK2, RIPK2, WA VE-2, TSC2, DAPKl, BAD, IMP, C-TAKI, TAKI, TAOl, TBKI, TESKI, TGFBRI, TIE2, TLKI, TrkA, TSSKI, TTBKI/2, TTK, Tpl2/cotl, MEKI, MEK2, PLDL Erkl, Erk2, Erk5, Erk8, p90RSK, PEA- 15, SRF, p27 KIPI, TIF 1a, HMGNI, ER81, MKP-3, c-Fos, FGF-Rl, GCK, GSK3 beta, HER4, HIPKI/2/3/, IGF-IR, cdc25, UBF, LAMTOR2, Statl, StaO, CREB, JAK, Src, SNCA, PTEN, NF- kappaB, HECTH9, Bax, HSP70, HSP90, Apaf-1, Cyto c, BCL-2, Bcl-xL, BCL-6, Smac, XIAP, Caspase-9, Caspase-3, Caspase-6, Caspase-7, CDC37, TAB, IKK, TRADD, TRAF2, RIPI, FLIP, TAKI, JNKl/2/3, Lek, A-Raf, B-Raf, C-Raf, MOS, MLKl/3, MN ½, MSKl, MST2/3/4, MPSKI, MEKKl, ME K4, MEL, ASKI, MINK I, MKK 1/2/3/4/6/7, NE, 2a/6/7, NUAKI, OSRI, SAP, STK33, Syk, Lyn, PDKI, PHK, PIM 1/2/3, Ataxin- 1, mTORCl, MDM2, p21 Wafl, Cyclin Dl, Lamln A, Tpl2, Myc, catenin, Wnt, IKK-beta, IKKgamma, IKK-alpha, IKK-epsilon, ELK, p65Re1A, IRAKI, IRA 2, IRAK4, IRR, FADD, TRAF6, TRAF3, MKK3, MKK6, ROCK2, RSKI/2, SGK 1, SmMLCK, SIK2/3, ULKI/2, VEGFRI, WNK 1, YESI, ZAP70, MAP4K3, MAP4K5, MAPKlb, MAPKAP-K2 K3, p38, alpha/beta/delta/gamma MAPK, Aurora A, Aurora B, Aurora C, MCAK, Clip, MAPKAPK, FAK, MARK 1 1/2/3/4, Mucl, SHC, CXCR4, Gap-I, Myc, beta-catenin/TCF, Cbl, BRM, Mell, BRD2, BRD3, BRD4, AR, RAS, ErbB3, EGFR, IREI, HPKI, RIPK2, ERa, or PCAF/GCN5, including all variants, mutations, splice variants, indels and fusions thereof.

In some embodiments, a linear RNA described herein is used to promote lysosomal degradation of a substrate protein. Lysosomes are membrane-enclosed organelles that can contain an array of digestive enzymes to degrade their contents. Substrate proteins can be delivered to lysosomes, for example, via endocytosis, phagocytosis, autophagy, macroautophagy, microautophagy, chaperone-mediated autophagy, or the multivesicular body pathway. Endocytosis and phagocytosis can deliver substrate proteins from an extracellular environment to lysosomes, e.g., via processes initiated by ligation of one or more endocytic or phagocytic receptors. Autophagy can deliver substrate proteins from an intracellular environment to lysosomes. In macroautophagy, proteins can be sequestered in vesicles that form in the cytosol and then fuse with lysosomes to transfer their contents for degradation. In microautophagy, proteins can be trapped inside vesicles that form directly through the invagination of the lysosomal membrane. These vesicles can then pinch off into the lysosomal lumen for degradation. In chaperone-mediated autophagy, substrate protein proteins in the cytosol can be recognized through the binding of a constitutive chaperone, the heat shock-cognate protein of 70 KDa (hsc70), to a pentapeptide motif present in the substrate protein. After substrate protein binding, the substrate protein can be translocated into the lysosomal lumen and degraded. Multivesicular bodies (MVBs) are a specialized subset of endosomes that contain membrane-bound intraluminal vesicles. These vesicles can form by budding into the lumen of the MVB. Sorting mechanisms can determine which MVB contents can be degraded via fusion with lysosomes, and which MVB contents can be recycled to the plasma membrane.

In some embodiments, a linear RNA described herein is used to promote lysosomal degradation of a substrate protein. For example, a linear RNA comprises a first binding site that binds a substrate protein, and a second binding site that directs the substrate protein to a lysosome. A linear RNA of the disclosure binds, for example, a ubiquitin ligase adaptor, or a second substrate that is trafficked to lysosomes, e.g., p62/SQSTM-1/Sequestosome-1, neighbor of BRCA1 gene 1 (NBR1), HDAC6, ESCRT-0 complex, ESCRT-I complex, ESCRT-II complex, or ESCRT-III complex. In some embodiments, a linear RNA described herein is used to direct a substrate protein to a lysosome via endocytosis. For example, a linear RNA can comprise a first binding site that binds a substrate protein, and a second binding site that directs the substrate protein to an endocytic receptor. In some embodiments, a linear RNA described herein is used to direct a substrate protein to a lysosome via phagocytosis. For example, a linear RNA comprises a first binding site that binds a substrate protein, and a second binding site that directs the substrate protein to an phagocytic receptor. In some embodiments, a linear RNA described herein is used to direct a substrate protein to a lysosome via autophagy, e.g., via macroautophagy, microautophagy, chaperone-mediated autophagy, the multivesicular body pathway, or a combination thereof. For example, a linear RNA comprises a first binding site that binds a substrate protein, and a second binding site that binds a factor that modulates an autophagy pathway, e.g., is involved in initiating an autophagy pathway or sorting substrates to the lysosome.

In some embodiments, a linear RNA described herein is used to promote or inhibit nitrosylation of a substrate protein. For example, a linear RNA comprises a first binding site that binds a substrate protein, and a second binding site that binds a factor involved in nitrosylation. Examples of factors involved in nitrosylation include, but are not limited to, nitrosylases, denitrosylases, NOS1, NOS2, NOS3, nNOS, iNOS, eNOS, hemoglobin, cytoglobin, neuroglobin, cytochrome C, ceruloplasmin, thioredoxin, GAPDH, caspase 3, CDK5, glutathione, glypican 1, AE1, caspase 3, HDAC, SIRT-1, DNAPK, X-linked inhibitor of apoptosis, and dynamin-related protein 1.

In some embodiments, a linear RNA described herein is used to promote or inhibit acetylation of a substrate protein. For example, a linear RNA comprises a first binding site that binds a substrate protein, and a second binding site that binds an acetyltransferase or a deacetylase. A linear RNA of the disclosure binds, for example, a lysine acetyltransferase, a histone acetyltransferase, a deacetylase, or a combination thereof. For example a linear RNA of the disclosure binds one or more factors that modulate acetylation, including, but not limited to, proteins encoded by ATAT1, CLOCK, CREBBP, ELP3, EP300, ESCO1, ESCO2, GTF3C4, HAT1, KAT14, KAT2A, KAT2B, MCM3AP, NCOA1, NCOA2, NCOA3, TAF1, HDAC1, HDAC2, HDAC3, HDAC4, HDAC5, HDAC6, HDAC7, HDAC8, HDAC8, HDAC9, HDAC10, HDAC11, SIRT1, SIRT2, SIRT3, SIRT4, SIRT5, SIRT6, and SIRT7.

In some embodiments, a linear RNA described herein is used to promote or inhibit SUMOylation of a substrate protein. For example, a linear RNA comprises a first binding site that binds a substrate protein, and a second binding site that binds a factor that modulates SUMOylation. For example, a linear RNA of the disclosure binds one or more factors that modulate SUMOylation, including, but not limited to, SAE1, SAE2, UBA2, UBE2I, SUMO1, SUM02, SUMO3, SUM04, Senp, Ubc9, proteins encoded by the aforementioned genes, or a combination thereof.

In some embodiments, a linear RNA described herein is used to promote or inhibit methylation of a substrate protein. For example, a linear RNA comprises a first binding site that binds a substrate protein, and a second binding site that binds a methyltransferase. A linear RNA of the disclosure binds, for example, a seven beta-strand methyltransferase, a SET methyltransferase, a SPOUT methyltransferase, a radical SAM methyltransferase, a MetH activating methyltransferase, a homocysteine methyltransferase, a membrane methyltransferase, a precorrin-like methyltransferrase, a TYW3 methyltransferase, a demethylase, or a combination thereof. For example a linear RNA of the disclosure binds one or more factors that modulate methylation, including, but not limited to, proteins encoded by AS3MT, ASH1L, ASMT, ASMTL, ATPSCKMT, BCDIN3D, BMT2, BUD23, CAMKMT, CARNMT1, CIAPIN1, CMTR1, CMTR2, COMT, COMTD1, COQ3, COQ5, DIMT1, DNMT1, DOT1L, DOT1L, EEF1AKMT1, EEF2KMT, EHMT1, EHMT2, EZH1, EZH2, FAM173A, FAM86B1, FAM86B2, FASN, FBL, FBLL1, FTSJ1, FTSJ3, GAMT, GNMT, GSTCD, HEMK1, HENMT1, HNMT, INMT, KMT2A, KMT2B, KMT2C, KMT2D, KMT2E, KMT5A, KMT5B, KMT5C, LCMT1, LCMT2, MECOM, MEPCE, MRM2, N6AMT1, NDUFAF5, NDUFAF7, NNMT, NSD1, NSD2, NSD3, PCMT1, PCMTD1, PCMTD2, PNMT, PRDM16, PRDM2, PRDM6, PRDM8, PRDM9, RNMT, RRP8, SETD1A, SETD1B, SETD2, SETD7, SETDB1, SETDB2, SMYD1, SMYD2, SMYD3, SUV39H1, SUV39H2, TFB1M, TFB2M, TGS1, THUMPD2, THUMPD3, TPMT, TRDMT1, ZCCHC4, KDM1A, JMJD1C, KDM1B, KDM2A, KDM2B, KDM3A, KDM3B, KDM4A, KDM4B, KDM4C, KDM4D, KDM4E, KDM4F, KDM5A, KDM5B, KDM5C, KDM5D, KDM6A, KDM6B, KDM7A, KDM8, PHF2, PHF8, and UTY.

In some embodiments, a linear RNA described herein is used to promote or inhibit phosphorylation of a substrate protein. For example, a linear RNA comprises a first binding site that binds a substrate protein, and a second binding site that binds a kinase or a phosphatase. A linear RNA of the disclosure binds, for example, a kinase, a protein kinase, a serine/threonine kinase, a tyrosine kinase, a receptor tyrosine kinase, a lipid kinase, a phosphatidylinositol kinase, a sphingosine kinase, a carbohydrate kinase, a thymidine kinase, a histidine kinase, a phosphatase, a tyrosine phosphatase, a serine/threonine phosphatase, a dual-specificity phosphatase, a histidine phosphatase, a phosphoprotein protein phosphatase, a lipid phosphatase, a haloacid dehalogenase, or a combination thereof. For example a linear RNA of the disclosure binds one or kinases, including, but not limited to, A6, A6ps1, A6ps2, A6r, AAK1, ABL, ACK, ACTR2, ACTR2B, ADCK1, ADCK2, ADCK3, ADCK4, ADCK5, AKT1, AKT2, AKT3, ALK, ALK1, ALK2, ALK4, ALK7, AlphaK1, AlphaK2, AlphaK3, AMPKa1, AMPKa2, ANKRD3, ANPa, ANPb, ARAF, ARAFps, ARG, ATM, ATR, AurA, AurAps1, AurAps2, AurB, AurBps1, AurC, AXL, BARK1, BARK2, BCKDK, BCR, BIKE, BLK, BMPR1A, BMPR1Aps1, BMPR1Aps2, BMPR1B, BMPR2, BMX, BRAF, BRAFps, BRD2, BRD3, BRD4, BRDT, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, ChaK1, ChaK2, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DNAPK, DRAK1, DRAK2, DYRK1A, DYRK1B, DYRK2, DYRK3, DYRK4, eEF2K, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FASTK, FER, FERps, FES, FGFR1, FGFR2, FGFR3, FGFR4, FGR, FLT1, FLT1ps, FLT3, FLT4, FMS, FRAP, FRK, Fused, FYN, G11, GAK, GCK, GCN2, GPRK4, GPRK5, GPRK6, GPRK6ps, GPRK7, GSK3A, GSK3B, H11, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK2, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDHK1, PDHK2, PDHK3, PDHK4, PDK1, PEK, PFTAIRE1, PFTAIRE2, PHKg1, PHKg1ps1, PHKglps2, PHKglps3, PHKg2, PI3K, PI4K2A, PI4KB, PIK3C2A, PIK3C2B, PIK3C2G, PIK3C2G, PIK3C3, PIK3CA, PIK3CG, PIK3R4, PIM1, PIM2, PIM3, PINK1, PIP4K2A, PIP5K1A, PIP5K1B, PIP5K1C, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIOK1, RIOK2, RIOK3, RIOK3ps, RIPK1, RIPK2, RIPK3, RNAseL, ROCK1, ROCK2, RON, ROR1, ROR2, ROS, RSK1, RSK2, RSK3, RSK4, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK, skMLCK, SLK, Slob, SMG1, smMLCK, SNRK, SPEG, SPHK1, SPHK2, SRC, SRM, SRPK1, SRPK2, SRPK2ps, SSTK, STK33, STK33ps, STLK3, STLKS, STLK6, STLK6ps1, STLK6-rs, SuRTK106, SYK, TAF1, TAF1L, TAK1, TAO1, TAO2, TAO3, TBCK, TBK1, TEC, TESK1, TESK2, TGFbR1, TGFbR2, TIE1, TIE2, TIF1a, TIF1b, TIF1g, TLK1, TLK1ps, TLK2, TLK2ps1, TLK2ps2, TNK1, Trad, Trb1, Trb2, Trb3, Trio, TRKA, TRKB, TRKC, TRRAP, TSSK1, TSSK2, TSSK3, TSSK4, TSSKps1, TSSKps2, TTBK1, TTBK2, TTK, TTN, TXK, TYK2, TYRO3, TYR03ps, ULK1, ULK2, ULK3, ULK4, VACAMKL, VRK1, VRK2, VRK3, VRK3ps, Wee1, Wee1B, Wee1Bps, Wee1ps1, Weelps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, ZC4/NRK, and proteins encoded by the aformentioned genes.

In some embodiments, a linear RNA of the disclosure binds one or phosphatases, including, but not limited to phosphatases encoded by ACP1, ANP32A, ANP32B, ANP32C, ANP32D, ANP32E, AUXI, BPNT1, CABIN1, CDC14A, CDC14B, CDC14C, CDC25A, CDC25B, CDC25C, CDKN3, CHP, CNEP1, CTDSPL2, CTPTP1, CTPTP2, CTPTPL, DOLPP1, DUPD1, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, EPM2A, EYA1, EYA2, EYA3, EYA4, GAK, HACD1, HDDC2, HDHD1A, HDHD2, HDHD3, ILKAP, IMPA1, IMPA2, IMPAD1, INPP1, INPP5A, INPP5B, INPP5D, INPP5E, INPP5F, INPPL1, ITPA, LHPP, LOC283871, LPPR1, LPPR2, LPPR3, LPPR4, MDSP, MINPP1, MTM1, MTMR1, MTMR10, MTMR11, MTMR12, MTMR2, MTMR3, MTMR4, MTMR6, MTMR7, MTMR8, MTMR9, NUDT10, NUDT11, NUDT14, NUDT3, NUDT4, OCRL, PAP2D, PDP2, PDXP, PHACTR1, PHACTR2, PHACTR3, PHACTR4, PIB5PA, PNKP, PPA1, PPA2, PPAP2A, PPAP2B, PPAP2C, PPAPDC1A, PPAPDC1B, PPAPDC2, PPAPDC3, PPEF1, PPEF2, PPM1A, PPM1B, PPM1D, PPM1E, PPM1F, PPM1G, PPM1H, PPM1J, PPM1K, PPM1L, PPM1M, PPM1N, PPP1CA, PPP1CB, PPP1CC, PPP1R11, PPP1R12A, PPP1R12B, PPP1R12C, PPP1R14A, PPP1R14B, PPP1R14C, PPP1R14D, PPP1R16A, PPP1R16B, PPP1R1A, PPP1R1B, PPP1R1C, PPP1R2, PPP1R3A, PPP1R3B, PPP1R3C, PPP1R3D, PPP1R3F, PPP1R7, PPP1R8, PPP2CA, PPP2CB, PPP2R1A, PPP2R1B, PPP2R2A, PPP2R2B, PPP2R2C, PPP2R2D, PPP2R3A, PPP2R3B, PPP2R3C, PPP2R4, PPP2R5A, PPP2R5B, PPP2R5C, PPP2R5D, PPP2R5E, PPP3CA, PPP3CB, PPP3CC, PPP3R1, PPP3R2, PPP4C, PPP4R1, PPP5C, PPP6C, PPTC7, PRG2, PSPH, PSPH, PTEN, PTEN2, PTN4, PTP4A1, PTP4A2, PTP4A3, PTPC1, PTPM1, PTPN1, PTPN11, PTPN12, PTPN13, PTPN14, PTPN18, PTPN2, PTPN20A, PTPN20B, PTPN21, PTPN22, PTPN23, PTPN3, PTPN5, PTPN6, PTPN7, PTPN9, PTPRA-1, PTPRA-2, PTPRB, PTPRC-1, PTPRC-2, PTPRD-1, PTPRD-2, PTPRE-1, PTPRE-2, PTPRF-1, PTPRF-2, PTPRG-1, PTPRG-2, PTPRH, PTPRJ, PTPRK-1, PTPRK-2, PTPRM-1, PTPRM-2, PTPRN, PTPRN2, PTPRO, PTPRQ, PTPRR, PTPRS-1, PTPRS-2, PTPRT-1, PTPRT-2, PTPRU-1, PTPRU-2, PTPRZ1-1, PTPRZ1-2, RNGTT, RP11, SACM1L, SAMHD1, SAPS1, SAPS2, SAPS3, SBF1, SBF2, SET, SGPP1, SGPP2, SKIP, SSH1, SSH2, SSH3, STYX, STYXL1, SYNJ1, SYNJ2, TENC1, TIMM50, TNS1, TNS3, TPTE, TPTE2, and UBLCP1.

In some embodiments, a linear RNA described herein is used to promote or inhibit glycosylation of a substrate protein. For example, a linear RNA comprises a first binding site that binds a substrate protein, and a second binding site that binds a glycosyltransferase, a glycoside hydrolase, or a sulfotransferase. For example a linear RNA of the disclosure binds one or more glycosyltransferases, including, but not limited to glycosyltransferases encoded by A3GALT2, A4GALT, A4GNT, ABO, ALG1, ALG10, ALG10B, ALG11, ALG12, ALG13, ALG14, ALG1L, ALG1L2, ALG2, ALG3, ALG5, ALG6, ALG8, ALG9, B3GALNT1, B3GALNT2, B3GALT1, B3GALT2, B3GALT4, B3GALT5, B3GALT6, B3GAT1, B3GAT2, B3GAT3, B3GLCT, B3GNT2, B3GNT3, B3GNT4, B3GNT5, B3GNT6, B3GNT7, B3GNT8, B3GNT9, B3GNTL1, B4GALNT1, B4GALNT2, B4GALNT3, B4GALNT4, B4GALT1, B4GALT2, B4GALT3, B4GALT4, B4GALT5, B4GALT6, B4GALT7, C1GALT1, CHPF, CHPF, CHPF2, CHPF2, CHSY1, CHSY1, CHSY3, CHSY3, COLGALT1, COLGALT2, CSGALNACT1, CSGALNACT2, DPM1, EOGT, EXT1, EXT2, EXTL1, EXTL2, EXTL3, FUT1, FUT10, FUT11, FUT2, FUT3, FUT4, FUT5, FUT6, FUT7, FUT8, FUT9, GALNT1, GALNT10, GALNT11, GALNT12, GALNT13, GALNT14, GALNT15, GALNT16, GALNT17, GALNT18, GALNT2, GALNT3, GALNT4, GALNT5, GALNT6, GALNT7, GALNT8, GALNT9, GALNTL5, GALNTL6, GBGT1, GCNT1, GCNT2, GCNT3, GCNT4, GCNT7, GLT1D1, GLT6D1, GLT8D1, GLT8D2, GTDC1, GXYLT1, GXYLT2, GYG1, GYG2, GYS1, GYS2, HAS1, HAS2, HAS3, LARGE1, LARGE2, LFNG, MFNG, MGAT1, MGAT2, MGAT3, MGAT4A, MGAT4B, MGAT4C, MGAT4D, MGAT5, MGAT5B, OGT, PIGA, PIGB, PIGM, PIGV, PIGZ, POFUT1, POFUT2, POGLUT1, POGLUT2, POGLUT3, POMGNT1, POMGNT2, POMT1, POMT2, PYGB, PYGL, PYGM, RFNG, RXYLT1, ST3GAL1, ST3GAL2, ST3GAL3, ST3GAL4, ST3GAL5, ST3GAL6, ST6GAL1, ST6GAL2, ST6GALNAC1, ST6GALNAC2, ST6GALNAC3, ST6GALNAC4, ST6GALNAC5, ST6GALNAC6, ST8SIA1, ST8SIA2, ST8SIA3, ST8SIA4, ST8SIA5, ST8SIA6, STT3A, STT3B, UGCG, UGGT1, UGGT2, UGT1A, UGT1A1, UGT1A10, UGT1A11P, UGT1A12P, UGT1A13P, UGT1A2P, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17, UGT2B24P, UGT2B25P, UGT2B26P, UGT2B27P, UGT2B28, UGT2B29P, UGT2B4, UGT2B7, UGT3A1, UGT3A2, UGT8, XXYLT1, XYLT1, and XYLT2.

In some embodiments, a linear RNA of the disclosure binds one or more glycoside hydrolases, including, but not limited to glycoside hydrolases encoded by AGL, AMY1A, AMY1B, AMY1C, AMY2A, AMY2B, AMYP1, CEMIP, CEMIP2, CHI3L1, CHI3L2, CHIA, CHID1, CHIT1, CTBS, EDEM1, EDEM2, EDEM3, FUCA1, FUCA2, GAA, GANAB, GANC, GBA3, GBE1, GLA, GLB1, GLB1L, GLB1L2, GLB1L3, HEXA, HEXB, HEXD, HPSE, HPSE2, HYAL1, HYAL2, HYAL3, HYAL4, HYAL6P, KL, KLB, LALBA, LCT, LCTL, LYG1, LYG2, LYZ, LYZL1, LYZL2, LYZL4, LYZL6, MAN1A1, MAN1A2, MAN1B1, MAN1C1, MAN2A1, MAN2A2, MAN2B1, MAN2B2, MAN2C1, MANBA, MANBAL, MANEA, MANEAL, MGAM, MGAM2, MYORG, NAGA, NEU1, NEU2, NEU3, NEU4, OGA, OVGP1, SI, SLC3A1, SLC3A2, SPACA3, SPACA5, SPACA5B, and SPAM1.

### Pharmaceutical Compositions

The present invention includes any compositions of linear RNA disclosed herein in combination with one or more pharmaceutically acceptable excipients. A pharmaceutically acceptable excipient can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions can optionally comprise one or more additional active substances, e.g., therapeutically and/or prophylactically active substances. Pharmaceutical compositions of the present invention can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005, which is incorporated herein by reference in its entirety. In one aspect, the invention includes a method of producing the pharmaceutical composition described herein comprising generating the linear polyribonucleotide.

Although the descriptions of pharmaceutical compositions provided herein are principally directed to pharmaceutical compositions which are suitable for administration to humans, it will be understood by the skilled artisan that such compositions are generally suitable for administration to any other animal, e.g., non-human animals and non-human mammals. Therefore, pharmaceutical compositions described herein can be used in therapeutic and veterinary. In some embodiments, pharmaceutical compositions (e.g., comprising a linear RNA as described herein) provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc..

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

### Delivery

The linear polyribonucleotide described herein may be included in pharmaceutical compositions comprising a pharmaceutically acceptable carrier or excipient. The linear polyribonucleotide described herein may be included in pharmaceutical compositions for delivery. The linear polyribonucleotide described herein can be included in pharmaceutical compositions with a delivery carrier. In some embodiments, the linear polyribonucleotide as described herein can be included in a pharmaceutical compoistion free of any carrier. In some embodiments, the linear polyribonucleotide as described can be included in a pharmaceutical composition comprising parenterally acceptable diluent. Methods as disclosed herein include a method of in vivo delivery of a linear polyribonucleotide as disclosed herein, composition as disclosed herein, or a pharmaceutical composition as disclosed herein comprising parenterally administering the linear polyribonucleotide, composition, or a pharmaceutical composition to the cell or tissue of a subject, or to a subject.

The methods of delivery as described herein comprise compositions of linear polyribonucleotides and methods of parenteral administration. A parenteral delivery system can comprise a linear polyribonucleotide and a parenterally acceptable diluent. In some embodiments, the delivery system is free of any carrier. In some embodiments, a composition, or pharmaceutical composition comprises the linear polyribonucleotide and parenterally acceptable diluent. In some embodiments, the composition or pharmaceutical composition further is free of any carrier.

Pharmaceutical compositions described herein may be formulated for example to include a pharmaceutical excipient or carrier. A pharmaceutical carrier can be a membrane, lipid bilayer, and/or a polymeric carrier, e.g., a liposome, such as a nanoparticle, e.g., a lipid nanoparticle, and delivered by known methods, such as via partial or full encapsulation of the linear polyribonucleotide, to a subject in need thereof (e.g., a human or non-human agricultural or domestic animal, e.g., cattle, dog, cat, horse, poultry). Such methods include, but are not limited to, transfection (e.g., lipid-mediated, cationic polymers, calcium phosphate, dendrimers); viral delivery (e.g., lentivirus, retrovirus, adenovirus, AAV), fugene, protoplast fusion, exosome-mediated transfer, lipid nanoparticle-mediated transfer, and any combination thereof. Cationic lipid-mediated delivery of proteins enables efficient protein-based genome editing in vitro and in vivo. Nat Biotechnol. 2014 Oct 30;33(1):73-80. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al.

Additional methods of delivery include electroporation (e.g., using a flow electroporation device) or other methods of membrane disruption (e.g., nucleofection), microinjection, microprojectile bombardment (“gene gun”), direct sonic loading, cell squeezing, optical transfection, impalefection, magnetofection, and any combination thereof. A flow electroporation device, for example, comprises a chamber for containing a suspension of cells to be electorporated, such as the cells (e.g., isolated cells) as described herein, the chamber being at least partially defined by oppositely chargeable electrodes, wherein the thermal resistance of the chamber is less than approximately 110° C. per Watt.

In some embodiments, the linear polyribonucleotide or a pharmaceutical composition is delivered as a naked delivery formulation. A naked delivery formulation delivers a linear polyribonucleotide to a cell without the aid of a carrier and without covalent modification or partial or complete encapsulation of the linear polyribonucleotide.

A naked delivery formulation is a formulation that is free from a carrier and wherein the linear polyribonucleotide is without a covalent modification that binds a moiety that aids in delivery to a cell or is without partial or complete encapsulation of the linear polyribonucleotide. In some embodiments, a linear polyribonucleotide without covalent modification bound to a moiety that aids in delivery to a cell is not covalently bound to a protein, small molecule, a particle, a polymer, or a biopolymer that aids in delivery to a cell. A linear polyribonucleotide without a covalent modification that binds a moiety that aids in delivery to a cell does not, for example, contain a modified phosphate group, such as phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[ 1 -(2,3-dioleoyloxy)propyl]-N,N,N- trimethylammonium chloride (DOTMA), 1-[2-(oleoyloxy)ethyl]-2-oleyl-3-(2-hydroxyethyl)imidazolinium chloride (DOTIM), 2,3-dioleyloxy-N- [2(sperminecarboxamido)ethyl]-N,N-dimethyl-1-propanaminium trifluoroacetate (DOSPA), 3B-[N- (N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N- hydroxyethyl ammonium bromide (DMRIE), N,N-dioleyl-N,N-dimethylammonium chloride (DODAC), human serum albumin (HSA), low-density lipoprotein (LDL), high- density lipoprotein (HDL), or globulin.

A naked delivery formulation may comprise a non-carrier excipient. In some embodiments, a non-carrier excipient may comprise an inactive ingredient. In some embodiments, a non-carrier excipient may comprise a buffer, for example PBS. In some embodiments, a non-carrier excipient may be a solvent, a non-aqueous solvent, a diluent (e.g., a parenterally acceptable diluent), a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent (e.g., a parenterally acceptable diluent). A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

The invention is further directed to a host or host cell comprising the linear polyribonucleotide described herein. In some embodiments, the host or host cell is a plant, insect, bacteria, fungus, vertebrate, mammal (e.g., human), or other organism or cell.

In some embodiments, the linear polyribonucleotide is non-immunogenic in the host. In some embodiments, the linear polyribonucleotide has a decreased or fails to produce a response by the host’s immune system as compared to the response triggered by a reference compound, e.g., a linear polyribonucleotide lacking an encryptogen. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., delivered to or administered to) the linear polyribonucleotide. In some embodiments, the host is a mammal, such as a human. The amount of the linear polyribonucleotide, expression product, or both in the host can be measured at any time after administration. In certain embodiments, a time course of host growth in a culture is determined. If the growth is increased or reduced in the presence of the circular polyribonucleotide, expression product, or both is identified as being effective in increasing or reducing the growth of the host.

### Methods of Delivery

A method of delivering a linear polyribonucleotide as described herein or a composition thereof as described herein to a cell, tissue or subject, comprises parenterally administering the circular polyribonucleotide or a composition thereof as described herein to the cell or tissue of a subject, or to a subject.

In some embodiments, the method of delivering is an in vivo method. For example, a method of delivering a linear polyribonucleotide as described herein comprises parenterally administering to a subject in need thereof, the pharmaceutical composition as described herein to the subject in need thereof. In some embodiments, the linear polyribonucleotide is an amount effective to have a biological effect on the cell or tissue in the subject. In some embodiments, the pharmaceutical composition as described herein comprises a carrier. In some embodiments the pharmaceutical composition as described herein comprises a diluent and is free of any carrier. In some embodiments, parenteral administration is intravenously. In some embodiments, parenteral administration is intramuscularly. In some embodiments, parenteral administration is ophthalmically. In some embodiments, parenteral administration is topically.

In some embodiments the linear polyribonucleotide or a composition thereof is administered parenterally. In some embodiments, the linear polyribonucleotide, composition thereof, or pharmaceutical composition thereof is administered orally. In some embodiments, the linear polyribonucleotide, composition thereof, or pharmaceutical composition thereof is administered nasally. In some embodiments, the linear polyribonucleotide, composition thereof, or pharmaceutical composition thereof is administered by inhalation. In some embodiments, the linear polyribonucleotide, composition thereof, or pharmaceutical composition thereof is administered topically. In some embodiments, the linear polyribonucleotide, composition thereof, or pharmaceutical composition thereof is administered opthalmically. In some embodiments the linear polyribonucleotide, composition thereof, or pharmaceutical composition thereof is administered rectally. In some embodiments the linear polyribonucleotide, composition thereof, or pharmaceutical composition thereof is administered by injection. The administration can be systemic administration. The administration can be local administration. In some embodiments, the linear polyribonucleotide, composition thereof, or pharmaceutical composition thereof is administered intravenously. In some embodiments, the linear polyribonucleotide, composition thereof, or pharmaceutical composition thereof is administered intraarterially. In some embodiments, the linear polyribonucleotide, composition thereof, or pharmaceutical composition thereof is administered intraperotoneally. In some embodiments, the linear polyribonucleotide, composition thereof, or pharmaceutical composition thereof is administered intradermally. In some embodiments, the linear polyribonucleotide, composition thereof, or pharmaceutical composition thereof is administered intracranially. In some embodiments, the linear polyribonucleotide, composition thereof, or pharmaceutical composition thereof is administered intrathecally. In some embodiments, the linear polyribonucleotide, composition thereof, or pharmaceutical composition thereof is administered intralymphaticly. In some embodiments, the linear polyribonucleotide, composition thereof, or pharmaceutical composition thereof is administered subcutaneously. In some embodiments, the linear polyribonucleotide, composition thereof, or pharmaceutical composition thereof is administered intramuscularly. In some embodiments, the linear polyribonucleotide, composition thereof, or pharmaceutical composition thereof is administered via intraocular administration. In some embodiments, the linear polyribonucleotide, composition thereof, or pharmaceutical composition thereof is administered via intracochlear (inner ear) administration. In some embodiments, the linear polyribonucleotide, composition thereof, or pharmaceutical composition thereof is administered viaintratracheal administration. In some embodiments, any of the methods of delivery as described herein are performed with a carrier. In some embodiments, the linear polyribonucleotide, composition thereof, or pharmaceutical composition thereof is administered intraarterially with a carrier. In some embodiments, the linear polyribonucleotide, composition thereof, or pharmaceutical composition thereof is administered intraperotoneally with a carrier. In some embodiments, the linear polyribonucleotide, composition thereof, or pharmaceutical composition thereof is administered intradermally with a carrier. In some embodiments, the linear polyribonucleotide, composition thereof, or pharmaceutical composition thereof is administered intracranially with a carrier. In some embodiments, the linear polyribonucleotide, composition thereof, or pharmaceutical composition thereof is administered intrathecally with a carrier. In some embodiments, the linear polyribonucleotide, composition thereof, or pharmaceutical composition thereof is administered intralymphaticly with a carrier. In some embodiments, the linear polyribonucleotide, composition thereof, or pharmaceutical composition thereof is administered subcutaneously with a carrier. In some embodiments, the linear polyribonucleotide, composition thereof, or pharmaceutical composition thereof is administered with a carrier. In some embodiments, the linear polyribonucleotide, composition thereof, or pharmaceutical composition thereof is administered intramuscularly with a carrier. In some embodiments, the linear polyribonucleotide, composition thereof, or pharmaceutical composition thereof is administered via intraocular administration with a carrier. In some embodiments, the linear polyribonucleotide, composition thereof, or pharmaceutical composition thereof is administered via intracochlear (inner ear) administration with a carrier. In some embodiments, the linear polyribonucleotide, composition thereof, or pharmaceutical composition thereof is administered via intratracheal administration with a carrier. In some embodiments, any methods of delivery as described herein are performed without the aid of a carrier in a naked delivery formulation. In some embodiments, the linear polyribonucleotide, composition thereof, or pharmaceutical composition thereof is administered intravenously without the aid of a carrier in a naked delivery formulation. In some embodiments, the linear polyribonucleotide, composition thereof, or pharmaceutical composition thereof is administered intrarterially without the aid of a carrier in a naked delivery formulation. In some embodiments, the linear polyribonucleotide, composition thereof, or pharmaceutical composition thereof is administered intraperotoneally without the aid of a carrier in a naked delivery formulation. In some embodiments, the linear polyribonucleotide, composition thereof, or pharmaceutical composition thereof is administered intradermally without the aid of a carrier in a naked delivery formulation. In some embodiments, the linear polyribonucleotide, composition thereof, or pharmaceutical composition thereof is administered intracranially without the aid of a carrier in a naked delivery formulation. In some embodiments, the linear polyribonucleotide, composition thereof, or pharmaceutical composition thereof is administered intrathecally without the aid of a carrier in a naked delivery formulation. In some embodiments, the linear polyribonucleotide, composition thereof, or pharmaceutical composition thereof is administered intralymphaticly without the aid of a carrier in a naked delivery formulation. In some embodiments, the linear polyribonucleotide, composition thereof, or pharmaceutical composition thereof is administered subcutaneously without the aid of a carrier in a naked delivery formulation. In some embodiments, the linear polyribonucleotide, composition thereof, or pharmaceutical composition thereof is administered without the aid of a carrier in a naked delivery formulation. In some embodiments, the linear polyribonucleotide, composition thereof, or pharmaceutical composition thereof is administered intramuscularly without the aid of a carrier in a naked delivery formulation. In some embodiments, the linear polyribonucleotide, composition thereof, or pharmaceutical composition thereof is administered via intraocular administration without the aid of a carrier in a naked delivery formulation. In some embodiments, the linear polyribonucleotide, composition thereof, or pharmaceutical composition thereof is administered via intracochlear (inner ear) administration without the aid of a carrier in a naked delivery formulation. In some embodiments, the linear polyribonucleotide, composition thereof, or pharmaceutical composition thereof is administered via intratracheal administration without the aid of a carrier in a naked delivery formulation.

### Cell and Vesicle-Based Carriers

A linear polyribonucleotide, composition, or pharmaceutical composition as described herein can be administered to a cell in a vesicle or other membrane-based carrier.

In some embodiments, the linear polyribonucleotide, composition thereof, or pharmaceutical composition thereof is administered in or via a cell, vesicle or other membrane-based carrier. In some embodiments, the linear polyribonucleotide, composition thereof, or pharmaceutical composition thereof can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a circular polyribonucleotide molecule or the pharmaceutical composition thereof as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3- Trimethylammonium-Propane(DOTAP), N-[ 1 -(2,3-dioleoyloxy)propyl]-N,N,N- trimethylammonium chloride (DOTMA), 1-[2-(oleoyloxy)ethyl]-2-oleyl-3-(2- hydroxyethyl)imidazolinium chloride (DOTIM), 2,3-dioleyloxy-N- [2(sperminecarboxamido)ethyl]-N,N-dimethyl-1-propanaminium trifluoroacetate (DOSPA), 3B-[N- (N\N′-Dimethylaminoethane)-carbamoyl]Cholesterol Hydrochloride (DC-Cholesterol HC1), diheptadecylamidoglycyl spermidine (DOGS), N,N-distearyl-N,N- dimethylammonium bromide (DDAB), N-(1,2-dimyristyloxyprop-3-yl)-N,N-dimethyl-N- hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high- density lipoprotein (HDL), or globulin.

Exosomes can also be used as drug delivery vehicles for a linear polyribonucleotide or a pharmaceutical composition thereof described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a linear polyribonucleotide or a pharmaceutical composition thereof described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; US Pat. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver linear polyribonucleotide, composition thereof, or pharmaceutical composition thereof described herein.

Virosomes and virus-like particles (VLPs) can also be used as carriers to the linear polyribonucleotide, composition thereof, or pharmaceutical composition thereof described herein to targeted cells.

Plant nanovesicles and plant messenger packs (PMPs), e.g., as described in International Patent Publication Nos. WO2011097480, WO2013070324, WO2017004526, or WO2020041784 can also be used as carriers to deliver the linear RNA as described herein.

Microbubbles can also be used as carriers to deliver a linear polyribonucleotide molecule described herein. See, e.g., US7115583; Beeri, R. et al., Circulation. 2002 Oct 1;106(14):1756-1759; Bez, M. et al., Nat Protoc. 2019 Apr; 14(4): 1015-1026; Hernot, S. et al., Adv Drug Deliv Rev. 2008 Jun 30; 60(10): 1153-1166; Rychak, J.J. et al., Adv Drug Deliv Rev. 2014 Jun; 72: 82-93. In some embodiments, microbubbles are albumin-coated perfluorocarbon microbubbles.

Silk fibroin can also be used as a carrier to deliver a linear polyribonucleotide molecule described herein. See, e.g., Boopathy, A.V. et al., PNAS. 116.33 (2019): 16473-1678; and He, H. et al., ACS Biomater. Sci. Eng. 4.5(2018): 1708-1715.

### Applications

Linear polyribonucleotides described herein can be administered to a cell, tissue or subject in need thereof, e.g., to modulate cellular function, e.g., gene expression in the cell, tissue or subject. The invention also contemplates methods of modulating cellular function, e.g., gene expression, comprising administering to a cell, tissue or subject in need thereof a linear polyribonucleotide described herein. The administered linear polyribonucleotides can be modified linear polyribonucleotides. In some embodiments, the administered linear polyribonucleotides are completely modified linear polyribonucleotides. In some embodiments, the administered linear polyribonucleotides are hybrid modified linear polyribonucleotides. In other embodiments, the administered linear polyribonucleotides are unmodified linear polyribonucleotides. The administered linear polyribonucleotides can comprise a conjugation moiety.

A linear polyribonucleotide of the disclosure can be used for treating a disease or condition in a subject in need thereof. A disease or condition can be, for example, a hyperproliferative disease, a cancer, a neurodegenerative disease, a metabolic disorder, an inflammatory disorder, an infectious disease, a genetic disease, or a combination thereof. A cancer can be, for example, a solid tumor or a liquid tumor. A solid tumor can be reproductive tissue cancer. A reproductive tissue cancer can be prostate cancer or cervical cancer. A liquid tumor can be a lymphoma. A lymphoma can be a B cell lymphoma. In some embodiments, the circular polyribonucleotide of the disclosure is administered intravenously to treat the disease or condition. In some embodiments, the circular polyribonucleotide of the disclosure is administered by intratumoral injection to treat the cancer.

Linear polyribonucleotides described herein can be for use as a medicament or a pharmaceutical. In some embodiments, the composition of any one of the linear polyribonucleotides as described herein, or the pharmaceutical composition of the linear polyribonucleotides as described herein is for use in a method of treatment of a human or animal body. In some embodiments, the composition of any one of the linear polyribonucleotides as described herein, or the pharmaceutical composition of the linear polyribonucleotides as described herein is formulated for intravenous administration or intratumoral administration. In some embodiments, the composition of any one of the linear polyribonucleotides as described herein, or the pharmaceutical composition of the linear polyribonucleotides as described herein is for use in a method of treating a cancer or a hyperproliferative disease; a neurodegenerative disease; a metabolic disorder; an inflammatory disorder; an autoimmune disease; an infectious disease; or a genetic disease. In some embodiments, the composition of the linear polyribonucleotides as described herein, or the pharmaceutical composition of the linear polyribonucleotides as described herein is for use in a method of treating a solid tumor (e.g., a reproductive tissue cancer, e.g., cervical cancer or prostate cancer) or a liquid tumor (e.g., lymphoma, e.g., a B cell lymphoma).

In some embodiments, a use of the composition of any one of the linear polyribonucleotides as described herein is in the manufacture of a medicament or a pharmaceutical.

In some embodiments, a use of the composition of any one of the linear polyribonucleotides as described herein is in the manufacture of a medicament or a pharmaceutical for treating a human or animal body by therapy.

In some embodiments, a use of the composition of any one of the linear polyribonucleotides as described herein is in the manufacture of a medicament for treating a cancer or a hyperproliferative disease; a neurodegenerative disease; a metabolic disorder; an inflammatory disorder; an autoimmune disease; an infectious disease; or a genetic disease.

In some embodiments, a use of the composition of any one of the the linear polyribonucleotides as described herein is in the manufacture of a medicament for treating a solid tumor (e.g., a reproductive tissue cancer, e.g., cervical cancer or prostate cancer) or a liquid tumor (e.g., lymphoma, e.g., a B cell lymphoma).

Pharmaceutical compositions described herein can be administered for prophylactic and/or therapeutic treatments. In therapeutic applications, the compositions can be administered to a subject already suffering from a disease or condition, in an amount sufficient to cure or at least partially reduce the symptoms of the disease or condition, or to cure, heal, improve, ameliorate, or reduce the condition. Compositions can also be administered to lessen a likelihood of developing, contracting, or worsening a condition. Amounts effective for this use can vary based on the severity and course of the disease or condition, previous therapy, the subject’s health status, weight, and response to the drugs, and the judgment of the treating physician.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms.

Pharmaceutical compositions provided herein can be administered in conjunction with other therapies, for example, chemotherapy, radiation, surgery, anti-inflammatory agents, and selected vitamins. The other agents can be administered prior to, after, or concomitantly with the pharmaceutical compositions.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art can alternatively be used.

### Numbered Embodiments #1

A composition comprising a linear polyribonucleotide comprising a conjugation moiety, wherein the conjugation moiety conjugates the linear polyribonucleotide to a chemical compound (e.g., a small molecule) that binds a target protein to modulate a substrate protein.
A composition comprising a linear polyribonucleotide comprising a first conjugation moiety and a second conjugation moiety, wherein the first conjugation moiety conjugates the linear polyribonucleotide to a first chemical compound (e.g., a small molecule) that binds to a target protein that modulates a substrate protein and wherein the second conjugation moiety conjugates the linear polyribonucleotide to a second chemical compound that binds the substrate protein.
A composition comprising:
   a) a linear polyribonucleotide comprising a conjugation moiety; and
   b) a chemical compound that binds a target protein;
   wherein the linear polyribonucleotide is conjugated to the chemical compound by the conjugation moiety and the target protein modulates a substrate protein.
A composition comprising:
   a) a linear polyribonucleotide comprising a first conjugation moiety and a second conjugation moiety;
   b) a first chemical compound that binds a target protein; and
   c) a second chemical compound that binds a substrate protein;
   wherein the linear polyribonucleotide is conjugated to the first chemical compound by the first conjugation moiety, the linear polyribonucleotide is conjugated to the second chemical compound by the second conjugation moiety, and the target protein modulates the substrate protein.
A composition of any one of the preceding embodiments, further comprising the target protein bound to the chemical compound to form a complex.
A composition of any one of the preceding embodiments, further comprising the target protein bound to the first chemical compound and the substrate protein bound to the second chemical compound to form a complex.
A composition comprising:
   a) a linear polyribonucleotide comprising a conjugation moiety;
   b) a chemical compound; and
   c) a target protein that modulates a substrate protein;
   wherein the conjugation moiety is conjugated to the chemical compound and the chemical compound is bound to the target protein to form a complex.
A composition comprising:
   a) a linear polyribonucleotide comprising a conjugation moiety and a binding site;
   b) a chemical compound; and
   c) a target protein that modulates a substrate protein;
   wherein the binding site is bound to the target protein, the conjugation moiety is conjugated to the chemical compound, and the chemical compound is bound to the substrate protein to form a complex.
A composition comprising:
   a) a linear polyribonucleotide comprising a first conjugation moiety and a second conjugation moiety;
   b) a first chemical compound that binds a target protein;
   c) a second chemical compound that binds a substrate protein; and
   d) the target protein that modulates the substrate protein;
   wherein the first conjugation moiety is conjugated to the first chemical compound, the second conjugation moiety is conjugated to the second chemical compound, and the first chemical compound is bound to the target protein to form a complex.
A composition comprising:
   a) a linear polyribonucleotide comprising a conjugation moiety;
   b) a chemical compound that binds to a target protein that modulates a substrate protein;
   c) the target protein; and
   d) the substrate protein;
   wherein the conjugation moiety is conjugated to the chemical compound, the chemical compound is bound to the target protein, and the target protein is bound to the substrate protein to form a complex.
A composition comprising:
   a) a linear polyribonucleotide comprising a first conjugation moiety and a second conjugation moiety;
   b) a first chemical compound that binds a target protein that modulates a substrate protein;
   c) a second chemical compound that binds the substrate protein;
   d) the target protein; and
   e) the substrate protein;
   wherein the first conjugation moiety is conjugated to the first chemical compound, the second conjugation moiety is conjugated to the second the chemical compound, the first chemical compound is bound to the target protein, and second chemical compound is bound to the substrate protein to form a complex.
The composition of any one of the preceding embodiments, wherein the linear polyribonucleotide further comprises a binding site that binds to the substrate protein or the target protein.
The composition of embodiment [12], wherein substrate comprises a linear polyribonucleotide (linear RNA)-binding motif.
A composition comprising:
   a) a first linear polyribonucleotide comprising a first conjugation moiety;
   b) a second linear polyribonucleotide comprising a second conjugation moiety;
   c) a first chemical compound that binds a target protein that modulates a substrate protein;
   d) a second chemical compound that binds the substrate protein;
   e) the target protein; and
   f) the substrate protein;
   wherein the first conjugation moiety is conjugated to the first chemical compound, the first chemical compound is bound to the target protein, the second conjugation moiety is conjugated to the second the chemical compound, and second chemical compound is bound to the substrate protein.
The composition of embodiment [14], wherein the substrate protein is bound to the target protein to form a complex.
The composition of any one of the preceding embodiments, wherein the conjugation moiety is a modified nucleotide.
The composition of any one of the preceding embodiments, wherein the first conjugation moiety is a first modified nucleotide and the second conjugation moiety is a second modified nucleotide.
The composition of embodiment [17], wherein the first modified nucleotide and second modified nucleotide are the same.
The composition of embodiment [17], wherein the first modified nucleotide and second modified nucleotide are different.
The composition of any one of the preceding embodiments, wherein the modified nucleotide is a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG.
The composition of any one of the preceding embodiments, wherein the first modified nucleotide is a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG, and the second modified nucleotide is a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG.
The composition of any one of the preceding embodiments, wherein the chemical compound or the first chemical compound is a small molecule.
The composition of any one of the preceding embodiments, wherein the chemical compound or the first chemical compound recruits the target protein.
The composition of any one of the preceding embodiments, wherein the chemical compound or the first chemical compound is a target protein ligand.
The composition of any one of the preceding embodiments, wherein the chemical compound or the first chemical compound is an LCL161 derivative, VHL-1, pomalidomide, lenalidomide, thalidomide or a derivative thereof, a HIF-1a-derived (R)-hydroxyproline, VHL ligand 2, VL-269, a VH032 derivative, or a hydroxyproline-based ligand.
The composition of any one the preceding embodiments, wherein the linear polyribonucleotide is conjugated to one or more additional chemical compounds.
The composition of embodiment [26], wherein the one or more additional chemical compounds are the same.
The composition of embodiment [26], wherein the one or more additional chemical compounds are different.
The composition of any one of the preceding embodiments, wherein the second chemical compound is a small molecule.
The composition of any one of the preceding embodiments, wherein the second chemical compound binds to a misfolded protein.
The composition of any one of the preceding embodiments, wherein the second chemical compound binds to a disease-associated protein.
The composition of any one of the preceding embodiments, wherein the second chemical compound binds to a protein associated with cancer.
The composition of any one of the preceding embodiments, wherein the second chemical compound binds to BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, or MDM2.
The composition of any one of the preceding embodiments, wherein the second chemical compound binds to GFP-halotag7.
The composition of any one of the preceding embodiments, wherein the second chemical compound is dasatinib, lapatinib, gefitinib, foretinib, Sirt2 inhibitor 3b, Sirt2 inhibitor, SNS-032, AC220, ceritinib, ibrutinib, ibrutinib dertivatie, 4-OHT, Jq1, PDE4 inhibitor, thiazolidinedione-based ligand, ripk2 inhibitor, bosutinib, OTX015, steel factor, TBK1 inhibitor, HJB97, aminopyrazole analog, RN486, AR antagonist, IACS-73, or nutlin small molecule.
The composition of any one of the preceding embodiments, wherein the second chemical compound is chloroalkane.
The composition of any one of the preceding embodiments, wherein the target protein is an enzyme.
The composition of any one of the preceding embodiments, wherein the target protein is a post-translational modification enzyme.
The composition of any one of the preceding embodiments, wherein the target protein modifies the substrate by adding a functional group to the substrate protein.
The composition of any one of the preceding embodiments, wherein the target protein modifies the substrate protein by acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, succinylation, sulfation, S-sulfenylation, S-sulfinylation, succinylation, sumoylation, or ubiquitination, uridylylation.
The composition of any one of the preceding embodiments, wherein the target protein is a ubiquitin ligase.
The composition of embodiment [41], wherein the ubiquitin ligase is a HECT, RING-finger, U-box, or PHD-finger ubiquitin ligase.
The composition of any one of embodiment [41] or [42], wherein the ubiquitin ligase is IAP, VHL, or CRBN.
The composition of any one of the preceding embodiments, wherein modulation of the substrate protein modulates a cellular process.
The composition of any one of the preceding embodiments, wherein degradation of the substrate protein modulates a cellular process.
The composition of any one of the preceding embodiments, wherein the cellular process is DNA damage repair, cell division, apoptosis, cell cycle regulation, signal transduction, transcriptional activity, or epigenetic regulation.
The composition of any one of the preceding embodiments, wherein the cellular process is associated with pathogenesis of a disease or condition.
The composition of any one of the preceding embodiments, wherein the substrate protein is a disease-associated protein.
The composition of any one of the preceding embodiments, wherein the substrate protein is a misfolded protein.
The composition of any one of the preceding embodiments, wherein the substrate protein comprises a mutation as compared to a wild-type version of the substrate protein.
The composition of any one of the preceding embodiments, wherein the substrate protein is BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, or MDM2.
The composition of any one of the preceding embodiments, wherein the complex alters substrate protein interactions with other proteins.
The composition of any one of the preceding embodiments, wherein the complex increases activity of the substrate protein.
The composition of any one of the preceding embodiments, wherein the complex decreases activity of the substrate protein.
The composition of any one of the preceding embodiments, wherein the complex alters localization of the substrate protein.
The composition of any one of the preceding embodiments, wherein the complex alters stability of the substrate protein.
The composition of any one of the preceding embodiments, wherein the complex promotes degradation of the substrate protein.
The composition of any one of the preceding embodiments, wherein the degradation of the substrate protein comprises proteasomal degradation.
The composition of any one of the preceding embodiments, wherein the complex promotes ubiquitination of the substrate protein.
The composition of any one of the preceding embodiments, wherein the linear polyribonucleotide comprises at least one modified nucleic acid.
The composition of any one of the preceding embodiments, wherein the at least one modified nucleic acid is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O-N-methylacetamido (2′-O-NMA), a locked nucleic acid (LNA), an ethylene nucleic acid (ENA), a peptide nucleic acid (PNA), a 1’,5′-anhydrohexitol nucleic acid (HNA), a morpholino, a methylphosphonate nucleotide, a thiolphosphonate nucleotide, and a 2′-fluoro N3-P5′-phosphoramidite.
The composition of any one of the preceding embodiments, wherein the linear polyribonucleotide comprises a cap.
The composition of any one for the preceding embodiments, wherein the linear polyribonucleotide comprises a UTR.
The composition of embodiment [63], wherein the UTR is a 5′ UTR.
The composition of embodiment [63], wherein the UTR is a 3′ UTR.
The composition of any one of the preceding embodiments, wherein the linear polyribonucleotide comprises a poly-A tail.
The composition of any one of the preceding embodiments, wherein the linear polyribonucleotide comprises a 5′ end protectant or a 3′ end protectant.
The composition of embodiment [67], wherein the 5′ end protectant or the 3′ end protectant protects the linear polyribonucleotide from degradation.
The composition of any one of embodiments [67] or [68], wherein the 5′ end protectant or the 3′ end protectant comprises a g-quadruplex, a pseudoknot, a stable terminal stem loop, a U-rich expression sequence, a nuclear retention element, a modified N terminus ribonucleic acid, a modified C terminus ribonucleic acid, a phosphodiester linkage modification, a sugar ring modification, 3′ end capping with inverted thymidine, or PEGylation.
The composition of any one of the preceding embodiments, wherein the linear polyribonucleotide comprises a protein binding site.
The composition of any one of the preceding embodiments, wherein the linear polyribonucleotide comprises an immunoprotein binding site.
The composition of any one of the preceding embodiments, wherein the linear polyribonucleic acid has a size of about 20 bases to about 20 kb.
The composition of any one of the preceding embodiments, wherein the linear polyribonucleic acid comprises one or more expression sequences.
A pharmaceutical composition comprising the composition of any one of the preceding embodiments and a pharmaceutically acceptable carrier or excipient.
A method of modulating a substrate protein, the method comprising:
   providing the composition or the pharmaceutical composition of any one of preceding embodiments; and
   administering the composition or the pharmaceutical composition to a subject having the substrate protein.
A method of treating a condition in a subject in need thereof, the method comprising administering to the subject the composition or the pharmaceutical composition of any one of the preceding embodiments.
The method of embodiment [76], wherein the condition is a cancer or a hyperproliferative disease.
The method of embodiment [76], wherein the condition is a neurodegenerative disease.
The method of embodiment [76], wherein the condition is a metabolic disorder.
The method of embodiment [76], wherein the condition is an inflammatory disorder.
The method of embodiment [76], wherein the condition is an autoimmune disease.
The method of embodiment [76], wherein the condition is an infectious disease.
The method of embodiment [76], wherein the condition is a genetic disease.

### Numbered Embodiments #2

A composition comprising a linear polyribonucleotide comprising a conjugation moiety, wherein the conjugation moiety conjugates the linear polyribonucleotide to a chemical compound (e.g., a small molecule) that binds a target protein to modulate a substrate protein.
A composition comprising a linear polyribonucleotide comprising a conjugation moiety, wherein the conjugation moiety conjugates the linear polyribonucleotide to a chemical compound (e.g., a small molecule) that binds a substrate protein for modulation.
A composition comprising a linear polyribonucleotide comprising a first conjugation moiety and a second conjugation moiety, wherein the first conjugation moiety conjugates the linear polyribonucleotide to a first chemical compound (e.g., a small molecule) that binds to a target protein that modulates a substrate protein and wherein the second conjugation moiety conjugates the linear polyribonucleotide to a second chemical compound that binds the substrate protein.
A composition comprising a linear polyribonucleotide comprising a conjugation moiety and a binding site, wherein the conjugation moiety conjugates the linear polyribonucleotide to a first chemical compound (e.g., a small molecule) that binds to a target protein that modulates a substrate protein and wherein the binding site (e.g., an aptamer) binds the substrate protein; or the conjugation moiety conjugates the linear polyribonucleotide to a first chemical compound (e.g., a small molecule) that binds to a substrate protein and wherein the binding site (e.g., an aptamer) binds a target protein that modulcates the substrate protein.
A composition comprising:
   a linear polyribonucleotide comprising a conjugation moiety; and
   a chemical compound that binds a target protein;
   wherein the linear polyribonucleotide is conjugated to the chemical compound by the conjugation moiety and the target protein modulates a substrate protein.
A composition comprising:
   a linear polyribonucleotide comprising a conjugation moiety; and
   a chemical compound that binds a substrate protein;
   wherein the linear polyribonucleotide is conjugated to the chemical compound by the conjugation moiety.
A composition comprising:
   a linear polyribonucleotide comprising a first conjugation moiety and a second conjugation moiety;
   a first chemical compound that binds a target protein; and
   a second chemical compound that binds a substrate protein;
   wherein the linear polyribonucleotide is conjugated to the first chemical compound by the first conjugation moiety, the linear polyribonucleotide is conjugated to the second chemical compound by the second conjugation moiety, and the target protein modulates the substrate protein.
A composition comprising:
   a linear polyribonucleotide comprising a conjugation moiety and a binding site;
   a chemical compound that binds a target protein;
   wherein the linear polyribonucleotide is conjugated to the first chemical compound by the first conjugation moiety, the binding site binds to the substrate, and the target protein modulates the substrate protein.
A composition comprising:
   a linear polyribonucleotide comprising a conjugation moiety and a second conjugation moiety;
   a first chemical compound that binds a target protein; and
   a second chemical compound that binds a substrate protein;
   wherein the linear polyribonucleotide is conjugated to the first chemical compound by the first conjugation moiety, the linear polyribonucleotide is conjugated to the second chemical compound by the second conjugation moiety, and the target protein modulates the substrate protein.
A composition of any one of the preceding embodiments, further comprising the target protein bound to the chemical compound to form a complex.
A composition of any one of the preceding embodiments, further comprising the target protein bound to the first chemical compound and the substrate protein bound to the second chemical compound to form a complex.
A composition comprising:
   a linear polyribonucleotide comprising a conjugation moiety;
   a chemical compound; and
   a target protein that modulates a substrate protein;
   wherein the conjugation moiety is conjugated to the chemical compound and the chemical compound is bound to the target protein to form a complex.
A composition comprising:
   a linear polyribonucleotide comprising a conjugation moiety and a binding site;
   a chemical compound; and
   a target protein that modulates a substrate protein;
   wherein the binding site is bound to the target protein, the conjugation moiety is conjugated to the chemical compound, and the chemical compound is bound to the substrate protein to form a complex.
A composition comprising:
   a linear polyribonucleotide comprising a first conjugation moiety and a second conjugation moiety;
   a first chemical compound that binds a target protein;
   a second chemical compound that binds a substrate protein; and
   the target protein that modulates the substrate protein;
   wherein the first conjugation moiety is conjugated to the first chemical compound, the second conjugation moiety is conjugated to the second chemical compound, and the first chemical compound is bound to the target protein to form a complex.
A composition comprising:
   a linear polyribonucleotide comprising a conjugation moiety;
   a chemical compound that binds to a target protein that modulates a substrate protein;
   the target protein; and
   the substrate protein;
   wherein the conjugation moiety is conjugated to the chemical compound, the chemical compound is bound to the target protein, and the target protein is bound to the substrate protein to form a complex.
A composition comprising:
   a linear polyribonucleotide comprising a first conjugation moiety and a second conjugation moiety;
   a first chemical compound that binds a target protein that modulates a substrate protein;
   a second chemical compound that binds the substrate protein;
   the target protein; and
   the substrate protein;
   wherein the first conjugation moiety is conjugated to the first chemical compound, the second conjugation moiety is conjugated to the second the chemical compound, the first chemical compound is bound to the target protein, and second chemical compound is bound to the substrate protein to form a complex.
A composition comprising a linear polyribonucleotide comprising a conjugation moiety, and a chemical compound that binds a substrate, wherein the conjugation moiety is conjugated to the chemical compound and the chemical compound is bound to the substrate protein.
The composition of embodiment [17], wherein the linear polyribonucleotide comprises at least 1, 2, 3, 4, 5, 6, 7, 8, 9, 10, 11, 12, 13, 14, 15, 20, 25, 30, 35, 40, 45, 50, 60, 70, 80, 90, or 100 conjugation moieties.
The composition of embodiment [17] or embodiment [18], the linear polyribonucleotide comprises at least, 2, 3, 4, 5, 6, 7, 8, 9, 10, 11, 12, 13, 14, 15, 20, 25, 30, 35, 40, 45, 50, 60, 70, 80, 90, or 100 chemical compounds.
The composition of any one embodiments [1]-[19], wherein the linear polyribonucleotide further comprises a binding site that binds to the substrate protein or the target protein.
A composition comprising a linear polyribonucleotide comprising a first conjugation moiety; a second conjugation moiety; a first chemical compound that binds a target protein that modulates a substrate protein; a second chemical compound that binds the substrate protein, wherein the first conjugation moiety is conjugated to the first chemical compound, the first chemical compound is bound to the target protein, the second conjugation moiety is conjugated to the second the chemical compound, and second chemical compound is bound to the substrate protein.
A composition comprising a linear polyribonucleotide comprising a conjugation moiety; a binding site that binds to a substrate protein; a chemical compound that binds a target protein that modulates the substrate protein, wherein the conjugation moiety is conjugated to the chemical compound.
The composition of embodiments [21] or [22], wherein the composition further comprises the target protein and/or the substrate protein.
The composition of any one of embodiments [1]-, wherein substrate comprises a linear polyribonucleotide (linear RNA)-binding motif.
The composition of any one of embodiments [4], [8], [13], [20], or [22], wherein the binding site is an aptamer.
The composition of any one of embodiments [1]-[25], wherein the linear polyribonucleotide is an exogenous, synthetic linear polyribonucleotide.
The composition of any one of embodiments [1]-[26], wherein the linear polyribonucleotide lacks a poly-A sequence, a replication element, or both.
A composition comprising:
   a first linear polyribonucleotide comprising a first conjugation moiety;
   a second linear polyribonucleotid comprising a second conjugation moiety;
   a first chemical compound that binds a target protein that modulates asubstrate protein;
   a second chemical compound that binds the substrate protein;
   the target protein; and
   the substrate protein;
   wherein the first conjugation moiety is conjugated to the first chemical compound, the first chemical compound is bound to the target protein, the second conjugation moiety is conjugated to the second the chemical compound, and second chemical compound is bound to the substrate protein.
The composition of embodiment [28], wherein the substrate protein is bound to the target protein to form a complex.
The composition of any one of embodiments [1]-[29], wherein the conjugation moiety is a modified nucleotide.
The composition of any one of embodiments [1]-[30], wherein the first conjugation moiety is a first modified nucleotide and the second conjugation moiety is a second modified nucleotide.
The composition of embodiment [31], wherein the first modified nucleotide and second modified nucleotide are the same.
The composition of embodiment [31], wherein the first modified nucleotide and second modified nucleotide are different.
The composition of embodiment [30], wherein the modified nucleotide is a modified UTP analog, a modified ATP analog, modified CTP analog, or a modified GTP analog.
The composition of embodiment [34], wherein the modified nucleotide comprises a click chemistry moiety.
The composition of embodiments [34] or [35], wherein the modified UTP analog 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG.
The composition of embodiments [31] or [32], wherein the first modified nucleotide is a modified UTP analog, a modified ATP analog, modified CTP analog, or a modified GTP analog, and wherein the second modified nucleotide is a modified UTP analog, a modified ATP analog, modified CTP analog, or a modified GTP analog.
The composition of embodiment [37], wherein the first modified nucleotide comprises a first click chemistry moiety and the second modified nucleotide comprises a second click chemistry moiety.
The composition of embodiments [37] or [38], wherein the first a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG, and the second modified nucleotide is a modified UTP analog, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2’,3′-ddATP, 5-Azido-PEG4-CTP, 5-DBCO-PEG4-CTP, N6-Azidohexyl-3′-dATP, 5-azidopropyl-UTP or 5-DBCO-PEG4-dCpG.
The composition of any one embodiment [1]-[39], wherein the chemical compound or the first chemical compound is a small molecule.
The composition of any one embodiment [1]-[40], wherein the chemical compound or the first chemical compound recruits the target protein.
The composition of any one embodiment [1]-[41], wherein the chemical compound or the first chemical compound is a target protein ligand.
The composition of any one embodiment [1]-[42], wherein the chemical compound or the first chemical compound is an LCL161 derivative, VHL-1, pomalidomide, lenalidomide, thalidomide or a derivative thereof, a HIF-1a-derived (R)-hydroxyproline, VHL ligand 2, VL-269, a VH032 derivative, or a hydroxyproline-based ligand.
The composition of any one embodiment [1]-[43], wherein the linear polyribonucleotide is conjugated to one or more additional chemical compounds.
The composition of embodiment [44], wherein the one or more additional chemical compounds are the same.
The composition of embodiment [44], wherein the one or more additional chemical compounds are different.
The composition of any one of embodiments [3], [7], [9], [11], [14], [16], [21], or [28], wherein the second chemical compound is a small molecule.
The composition of any one of embodiments [3], [7], [9], [11], [14], [16], [21], [28], or [47], wherein the second chemical compound binds to a misfolded protein.
The composition of any one of embodiments [3], [7], [9], [11], [14], [16], [21], [28], or [47], wherein the second chemical compound binds to a disease-associated protein.
The composition of any one of embodiments [3], [7], [9], [11], [14], [16], [21], [28], or [47], wherein the second chemical compound binds to a protein associated with cancer.
The composition of any one of embodiments [3], [7], [9], [11], [14], [16], [21], [28], or [47], wherein the chemical compound or the second chemical compound is a Heat Shock Protein 90 (HSP90) inhibitor, Kinase and Phosphatase inhibitor, MDM2 inhibitor, HDAC inhibitor, Human Lysine Methyltransferase Inhibitor, Angiogenesis inhibitor, Immunosuppressive compound, or a compounds that binds to a Human BET Bromodomain-containing protein, the aryl hydrocarbon receptor (AHR), REF receptor kinase, FKBP, Androgen Receptor (AR), Estrogen receptor (ER), Thyroid Hormone Receptor, HIV Protease, HIV Integrase, HCV Protease, and Acyl-protein Thioesterase-1 and-2 (APTI and APT2).
The composition of any one of embodiments [3], [7], [9], [11], [14], [16], [21], [28], or [47], wherein the second chemical compound binds to BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, or MDM2.
The composition of any one of embodiments [3], [7], [9], [11], [14], [16], [21], [28], or [47], wherein the second chemical compound binds to GFP-halotag7.
The composition of any one of embodiments [3], [7], [9], [11], [14], [16], [21], [28], or [47], wherein the second chemical compound is dasatinib, lapatinib, gefitinib, foretinib, Sirt2 inhibitor 3b, Sirt2 inhibitor, SNS-032, AC220, ceritinib, ibrutinib, ibrutinib dertivatie, 4-OHT, Jq1, PDE4 inhibitor, thiazolidinedione-based ligand, ripk2 inhibitor, bosutinib, OTX015, steel factor, TBK1 inhibitor, HJB97, aminopyrazole analog, RN486, AR antagonist, IACS-73, or nutlin small molecule.
The composition any one of embodiments [3], [7], [9], [11], [14], [16], [21], [28], or [47], wherein the second chemical compound is chloroalkane.
The composition of any one of embodiments [1]-[55], wherein the target protein is an enzyme.
The composition of any one of embodiments [1]-[56], wherein the target protein is a post-translational modification enzyme.
The composition of any one of embodiments [1]-[57], wherein the target protein modifies the substrate by adding a functional group to the substrate protein.
The composition of any one of embodiments [1]-[58], wherein the target protein modifies the substrate protein by acetylation, acylation, adenylylation, ADP-ribosylation, alkylation, amidation, amide bond formation, amino acid addition, arginylation, beta-lysine addition, butyrylation, carbamidation, carbonylation, carboxylation, citrullination, C-linked glycosylation, crotonylation, diphthamide formation, deacetylation, demethylation, ethanolamine phosphoglycerol attachment, farnesylation, flavin moiety attachment, formylation, gamma-carboxyglutamic acid, gamma-carboxylation, geranilgeranilation, glutarylation, glutathionylation, glycosylation, GPI-anchor formation, heme C attachment, hydroxylation, hypusine formation, iodination, ISGylation, isoprenylation, lipoylation, malonylation, methylation, myristoylation, N-acylation, N-linked glycosylation, neddylation, nitration, nitrosylation, nucleotide addition, O-acylation, O-linked glycosylation, oxidation, palmitoylation, phosphate ester formation, phosphoramidate formation, phosphorylation, phosphopantetheinylation, polyglutamylation, polyglycylation, polysialylation, prenylation, propionylation, pyroglutamate formation, pyrrolidone carboxylic acid, pyrrolylation, pyruvate, Retinylidene Schiff base formation, S-acylation, S-diacylglycerol, S-glutathionylation, S-linked glycosylation, S-nitrosylation, succinylation, sulfation, S-sulfenylation, S-sulfinylation, succinylation, sumoylation, or ubiquitination, uridylylation.
The composition of any one of embodiments [1]-[59], wherein the target protein is a ubiquitin ligase.
The composition of embodiment [60], wherein the ubiquitin ligase is a HECT, RING-finger, U-box, or PHD-finger ubiquitin ligase.
The composition of any one of embodiment [60] or [61], wherein the ubiquitin ligase is selected from the group consisting of von Rippel-Lindau (VHL); cereblon; XIAP; E3A; MDM2; Anaphase-promoting complex (APC); UBR5 (EDDI); SOCS/ BC-box/ eloBC/ CUL5/ RING; LNXp80; CBX4; CBLLI; HACEI; HECTDI; HECTD2; HECTD3; HECWI; HECW2; HERCI; HERC2; HERC3; HERC4; HUWEI; ITCH; NEDD4; NEDD4L; PPIL2; PRPF19; PIASI; PIAS2; PIAS3; PIAS4; RANBP2; RNF4; RBXI; SMURFI; SMURF2; STUBI; TOPORS; TRIP12; UBE3A; UBE3B; UBE3C; UBE4A; UBE4B; UBOX5; UBR5; WWPI; WWP2; Parkin; A20/TNFAIP3; AMFR/gp78; ARA54; beta-TrCPI/BTRC; BRCAI; CBL; CHIP/STUB I; E6; E6AP/UBE3A; F-box protein 15/FBXOIS; FBXW7/Cdc4; GRAIL/RNF128; HOIP/RNF3 1; cIAP-⅟HIAP-2; cIAP- 2/HIAP-1; cIAP (pan); ITCH/AIP4; KAPI; MARCH8;; Mind Bomb ⅟MIBI; Mind Bomb 2/MIB2; MuRF1/TRIM63; NDFIPI; NEDD4; N1eL; Parkin; RNF2; RNF4; RNF8; RNF168; RNF43; SARTI; Skp2; SMURF2; TRAF-1; TRAF-2; TRAF-3; TRAF-4; TRAF-5; TRAF-6; TRIMS; TRIM21; TRIM32; UBR5; and ZNRF3.
The composition of any one of embodiments [1]-[62], wherein modulation of the substrate protein modulates a cellular process.
The composition of any one of embodiments [1]-[63], wherein degradation of the substrate protein modulates a cellular process.
The composition of any one of embodiments [1]-[64], wherein the cellular process is DNA damage repair, cell division, apoptosis, cell cycle regulation, signal transduction, transcriptional activity, or epigenetic regulation.
The composition of any one of embodiments [1]-[65], wherein the cellular process is associated with pathogenesis of a disease or condition.
The composition of any one of embodiments [1]-[66], wherein the substrate protein is a disease-associated protein.
The composition of any one of embodiments [1]-[67], wherein the substrate protein is a misfolded protein.
The composition of any one of embodiments [1]-[68], wherein the substrate protein comprises a mutation as compared to a wild-type version of the substrate protein.
The composition of any one of embodiments [1]-[69], wherein the substrate protein is BCR-Abl, c-ABL, EGFR, c-Met, Sirt2, CDK9, FLT3, ALK, BTK, ERalpha, BRD2/3/4, PDE4, ERRalpha, RIPK2, FKBP12, TBK1, BRD9, HER2, AR, TRIM23, MDM2, FoxOl, HDAC, DP-1, E2F, ABL, ALK, AMPK, BRK, BRSK I, BRSK2, BTK, CAMKKI, CAMKK alpha, CAMKK beta, Rb, Suv39HI, SCF, pl9INK4D, GSK-3, pi 8 INK4, myc, cyclin E, CDK2, CDK9, CDG4/6, Cycline D, p16 INK4A, cdc25A, BMII, SCF, Akt, CHK½, CI delta, CKI gamma, C 2, CLK2, CSK, DDR2, DYRKIA/2/3, EF2K, EPH-A2/A4/B1/B2/B3/B4, EIF2A 3, Smad2, Smad3, Smad4, Smad7, p53, p21 Cipl, PAX, Fyn, CAS, C3G, SOS, Tal, Raptor, RACK-I, CRK, Rapl, Rae, KRas, NRas, HRas, GRB2, FAK, PBK, spred, Spry, mTOR, MPK, LKB1, PAK 1/2/4/5/6, PDGFRA, PYK.2, Src, SRPKI, PLC, PKC, PKA, PKB, alpha/beta, PKC alpha/gamma/zeta, PKD, PLK1, PRAK, PRK2, RIPK2, WA VE-2, TSC2, DAPK1, BAD, IMP, C-TAKI, TAKI, TAO1, TBKI, TESKI, TGFBRI, TIE2, TLKI, TrkA, TSSKI, TTBKI/2, TTK, Tp12/cotl, MEKI, MEK2, PLDL Erk1, Erk2, Erk5, Erk8, p90RSK, PEA- 15, SRF, p27 KIPI, TIF 1a, HMGNI, ER81, MKP-3, c-Fos, FGF-R1, GCK, GSK3 beta, HER4, HIPKI/2/3/, IGF-IR, cdc25, UBF, LAMTOR2, Statl, StaO, CREB, JAK, Src, SNCA, PTEN, NF- kappaB, HECTH9, Bax, HSP70, HSP90, Apaf-1, Cyto c, BCL-2, Bcl-xL, BCL-6, Smac, XIAP, Caspase-9, Caspase-3, Caspase-6, Caspase-7, CDC37, TAB, IKK, TRADD, TRAF2, RIPI, FLIP, TAKI, JNK1/2/3, Lek, A-Raf, B-Raf, C-Raf, MOS, MLK1/3, MN 1/2, MSK1, MST2/3/4, MPSKI, MEKK1, ME K4, MEL, ASKI, MINK I, MKK 1 /2/3/4/6/7, NE, 2a/6/7, NUAKI, OSRI, SAP, STK33, Syk, Lyn, PDKI, PHK, PIM 1/2/3, Ataxin- 1, mTORC1, MDM2, p21 Waf1, Cyclin D1, Lamln A, Tp12, Myc, catenin, Wnt, IKK-beta, IKKgamma, IKK-alpha, IKK-epsilon, ELK, p65Re1A, IRAKI, IRA 2, IRAK4, IRR, FADD, TRAF6, TRAF3, MKK3, MKK6, ROCK2, RSKI/2, SGK 1, SmMLCK, SIK2/3, ULKI/2, VEGFRI, WNK 1, YESI, ZAP70, MAP4K3, MAP4K5, MAPK1b, MAPKAP-K2 K3, p38, alpha/beta/delta/gamma MAPK, Aurora A, Aurora B, Aurora C, MCAK, Clip, MAPKAPK, FAK, MARK 1 /2/3/4, Mucl, SHC, CXCR4, Gap-I, Myc, beta-catenin/TCF, Cbl, BRM, Mell, BRD2, BRD3, BRD4, AR, RAS, ErbB3, EGFR, IREI, HPKI, RIPK2, ERa, or PCAF/GCN5.
The composition of any one of embodiments [10]-[16], or [29], wherein the complex alters substrate protein interactions with other proteins.
The composition of any one of embodiments [10]-[16], or [29], wherein the complex increases activity of the substrate protein.
The composition of any one of embodiments [10]-[16], or [29], wherein the complex decreases activity of the substrate protein.
The composition of any one of embodiments [10]-[16], or [29], wherein the complex alters localization of the substrate protein.
The composition of any one of embodiments [10]-[16], or [29], wherein the complex alters stability of the substrate protein.
The composition of any one of embodiments [10]-[16], or [29], wherein the complex promotes degradation of the substrate protein.
The composition of any one of embodiments [10]-[16], or [29], wherein the degradation of the substrate protein comprises proteasomal degradation.
The composition of any one of embodiments [10]-[16], or [29], wherein the complex promotes ubiquitination of the substrate protein.
The composition of any one of embodiments [4], [8], [13], [20], [22], or [25], wherein the binding site is an aptamer.
The composition of any one of embodiments [4], [8], [13], [20], [22], or [25], wherein the binding site is a miRNA binding site.
The composition of any one of embodiments [1]-[80], wherein the linear polyribonucleotide is translation incompetent or translation defective.
The composition of any one of embodiments [1]-[81], wherein the linear polyribonucleotide further comprises at least one structural element selected from the group consisting of:
   an encryptogen;
   a splicing element;
   a regulatory sequence;
   a replication sequence;
   a quasi-double-stranded secondary structure;
   a quasi-helical structure; and
   an expression sequence.
The composition of embodiment [82], wherein the quasi-helical structure comprises at least one double-stranded RNA segment with at least one non-double-stranded segment.
The composition of embodiment [82], wherein the quasi-helical structure comprises a first sequence and a second sequence linked with a repetitive sequence.
The composition of embodiment [82], wherein the encryptogen comprises a splicing element.
The composition of embodiment [82], wherein the encryptogen comprises at least one modified nucleic acid.
The composition of embodiment [82], wherein the encryptogen comprises a protein binding site.
The composition of embodiment [82], wherein the encryptogen comprises an immunoprotein binding site.
The composition of any one of embodiments [1]-[88], wherein the linear polyribonucleic acid has at least 2x lower immunogenicity than a counterpart lacking the encryptogen, as assessed by expression, signaling, or activation of at least one of RIG-I, TLR-3, TLR-7, TLR-8, MDA-5, LGP-2, OAS, OASL, PKR, and IFN-beta.
The composition of any one of embodiments [1]-[89], wherein the linear polyribonucleic acid has a size of about 20 bases to about 20 kb.
The composition of any one of embodiments [1]-[90], wherein the linear polyribonucleic acid is synthesized through linearization of a linear polyribonucleotide.
The composition of any one of embodiments [1]-[91], wherein the linear polyribonucleic acid is substantially resistant to degradation.
The composition of any one of embodiments [1]-[92], wherein the linear polyribonucleic acid comprises one or more expression sequences.
A pharmaceutical composition comprising the composition of any one of the preceding embodiments and a pharmaceutically acceptable carrier or excipient.
A pharmaceutical composition comprising the composition of any one of the preceding embodiments and a pharmaceutically acceptable excipient and is free of any carrier.
A method of treating a condition in a subject in need thereof, the method comprising administering to the subject the composition or the pharmaceutical composition of any one of the preceding embodiments.
The method of embodiment [96], wherein the condition is a cancer or a hyperproliferative disease.
The method of embodiment [97], wherein the cancer is a solid tumor or a liquid tumor.
The method of embodiment [98], wherein the solid tumor is a reproductive tissue cancer (e.g., prostate cancer or cervical cancer).
The method of embodiment [98], wherein the liquid tumor is a lymphoma (e.g., B cell lymphoma).
The method of embodiment [96], wherein the condition is a neurodegenerative disease.
The method of embodiment [96], wherein the condition is a metabolic disorder.
The method of embodiment [96], wherein the condition is an inflammatory disorder.
The method of embodiment [96], wherein the condition is an autoimmune disease.
The method of embodiment [96], wherein the condition is an infectious disease.
The method of embodiment [96], wherein the condition is a genetic disease.
The method of any one of embodiments [96]-[106], wherein administering is intravenous administration.
The method of any one of embodiments [96]-[107], wherein administering is intratumoral administration.
A bifunctional linear polyribonucleotide, wherein the bifunctional linear polyribonucleotide comprises the following chemical structure:
   X₁- linear polyribonucleotide-X₂
   wherein X₁ and X₂ independently comprise a molecule comprising an E3 ubiquitin ligase binding moiety (UBM) or a molecule comprising a protein binding moiety (PBM).
The bifunctional linear ribonucleotide of embodiment [109], wherein X₁ comprises an UBM.
The bifunctional linear ribonucleotide of embodiment [109], wherein X₁ comprises an PBM.
The bifunctional linear ribonucleotide of any one of embodiments [109]-[111], wherein the PBM is a Von Hippel-Lindau E3 ubiquitin ligase binding moiety, a cereblon E3 ubiquitin ligase binding moiety, an MDM2 E3 ubiquitin ligase binding moiety, an IAP binding moiety, or a combination thereof.
The bifunctional linear ribonucleotide of any one of embodiments [109]-[112], wherein X₁ comprise one or more UBMs and one or more PBMs.
The bifunctional linear ribonucleotide of any one of embodiments [109]-[112], wherein X₂ comprise one or more UBMs and one or more PBMs.
The bifunctional linear ribonucleotide of any one of embodiments [109]-[114], wherein X₁ and X₂ independently comprise one or more UBMs and one or more PBMs.
The bifunctional linear ribonucleotide of any one of embodiments [109]-[115], wherein the one or more UBMs are identical.
The bifunctional linear ribonucleotide of any one of embodiments [109]-[115], wherein the one or more UBMs are different.
The bifunctional linear ribonucleotide of any one of embodiments [109]-[117], wherein the one or more PBMs are identical.
The bifunctional linear ribonucleotide of any one of embodiments [109]-[117], wherein the one or more UBMs are different.
The bifunctional linear ribonucleotide of any one of embodiments [109]-[119], wherein X₁ and X₂ independently comprise up to 100 binding moieties.

### EXAMPLES

### Example 1: Linear RNA That Contains a Chemical Compound

This Example describes linear RNA linked to a chemical compound to bind and recruit a protein of choice.

Thalidomide, a clinically approved drug (Thalomid), is known to associate with a member of the cells’ protein degradation machinery, the E3 ubiquitin ligase. By conjugating thalidomide to linear RNA (e.g., via click chemistry), thalidomide-conjugated linear RNA can recruit cells’ degradation machinery to a second, disease-causing protein (e.g., also targeted by the linear RNA). In the following Example, a chemical compound, e.g., a small molecule is conjugated to a linear RNA to bind E3 ubiquitin ligase Cereblon for ubiquitination and subsequent degradation of a target protein.

Linear RNA is designed to include reactive uridine residues (e.g., 5-azido-C3-UTP) for conjugation of alkyne-functionalized small molecules, known to interact with an intracellular protein of interest.

Linear RNA is synthesized by either in vitro transcription using T7 RNA polymerase (Lucigen) or custom synthesized by commercial entities. All UTPs are substituted with 5-azido-C3-UTP (Jena Biosciences) in the in vitro transcription reaction to generate azide-functionalized RNA. Synthesized linear RNA can comprise unmodified or modified bases. Synthesized linear RNA is purified with an RNA clean up kit (New England Biolabs) and is subjected to RNA 5′ Pyrophosphohydrolase (RppH, New England Biolabs) treatment to remove pyrophosphate. RppH-treated linear RNA iss purified with an RNA clean up kit (New England Biolabs).

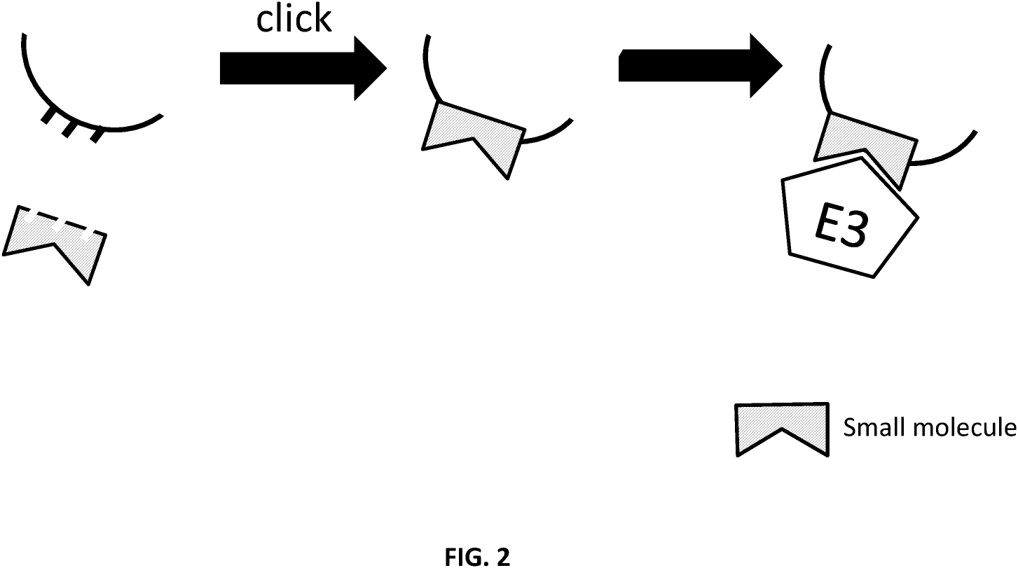

FIG. 2 illustrates click chemistry conjugation of a chemical compound to a linear RNA to generate a linear RNA that can bind an E3 ubiquitin ligase.

Alkyne-functionalized thalidomide (Jena Bioscience) is conjugated to azide-functionalized linear RNA via Copper-catalyzed Azide-Alkyne click chemistry reactions (CuAAC) with the click chemistry reaction kit based on manufacturer’s instructions (Jena Bioscience). Thalidomide-conjugated linear RNA iss purified with an RNA clean up kit (New England Biolabs).

Binding properties of the thalidomide-conjugated linear RNA binding is analyzed using GST pull-down followed by qPCR for RNA detection. For GST pull-down assay, thalidomide-conjugated linear RNA (2 nM) is incubated with GST-E3 ubiquitin ligase Cereblon (50 nM), which interacts with thalidomide, for 2 hours at room temperature in the presence of 25 mM Tris-Cl (pH7.0), 100 mM NaCl, 1 mM EDTA, 0.5% NP-40, 5% Glycerol. Azide-functionalized linear RNA without thalidomide conjugation is used as a negative control.

The RNA-protein mixture is further incubated for an hour at room temperature with GSH-agarose beads to assess GST-GSH interactions. After washing three times with binding buffer, the RNA specifically bound to the GSH-beads is extracted with Trizol (Thermo Fisher). The extracted linear RNA is reverse transcribed and detected by quantitative RT-PCR with primers specific for linear RNA.

Linear RNA conjugated to the thalidomide small molecule is highly enriched in the GST pull-down, demonstrating that a linear RNA-chemical compound conjugate is bound a target protein via the small molecule, e.g., to promote degradation of the protein of choice.

### Example 2: Linear RNA With a Chemical Compound

This Example describes linear RNA is linked to a chemical compound that induces specific bioactivity.

The following Example shows a chemical compound, e.g., a small molecule, is clicked to a linear RNA to harness specific bioactivity, e.g., ubiquitination, for bioactivity on a specific protein.

Linear RNA is designed to include reactive uridine residues (e.g., 5-azido-C3-UTP or 5-ethyl-UTP) for conjugation of alkyne-functionalized or azide-functionalized small molecules, for any downstream functionality.

Linear RNA is synthesized by either in vitro transcription using T7 RNA polymerase (Lucigen) or custom-synthesized by commercial entities. All UTP was substituted with 5-azido-C3-UTP or 5-ethyl-UTP (Jena Biosciences) in the in vitro transcription reaction to generate azide-functionalized or alkyne functionalized RNA, respectively. Synthesized linear RNA can comprise unmodified or modified bases. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Alkyne-functionalized Alex Fluor 488 dye or azide-functionalized Alex Fluor 488 dye (Jena Bioscience) is conjugated to azide-functionalized linear RNA or alkyne-funcitonalized RNA via Copper-catalyzed Azide-Alkyne click chemistry reactions (CuAAC) with the click chemistry reaction kit based on manufacturer’s instructions (Jena Bioscience). Alexa Fluor 488-conjugated linear RNA is purified with an RNA clean up kit (New England Biolab).

The dye conjugation is monitored by separating linear RNA on 6% denaturing UREA-PAGE. Alexa Fluore dye-unconjugated and -conjugated linear RNA were separated on the gel in parallel for comparison. Fluorescence from the RNA on the gel is monitored by iBright Imaging System (Invitrogen). After monitoring fluorescence, the gel is stained with SYBR safe and RNA on the gel is visualized by iBright Imaging System (Invitrogen).

Linear RNA containing a chemical compound Alexa Fluor 488 is shown to fluoresce showing that a functional chemical compound is contained in the linear RNA.

Using similar reaction, linear RNA is conjugated to the thalidomide small molecule. When run on a 6% denaturing UREA-PAGE gel, a discrete product band is produced and is separate from the unconjugated linear RNA. This is expected to show that linear RNA is conjugated to a chemical compound that interacts with a specific bioactive protein.

### Example 3: Linear RNA With Two Chemical Compounds

This example describes a linear RNA containing two conjugated chemical compounds that can each recruit a protein.

VH 032 is a small molecule known to associate with the E3 ubiquitin ligase VHL. A VH 032-conjugated linear RNA that also contains a binding site for a second target protein could therefore recruit an E3 ubiquitin ligase and the second target protein, for example, to target the second (e.g., disease-causing) protein for ubiquitination and degradation.

Gefitinib is a drug that is known to bind to the epidermal growth factor receptor (EGFR). A gefitinib-conjugated linear RNA that also binds to a ubiquitin ligase could be used, for example, to target EGFR for ubiquitination and degradation.

In the following example, a linear RNA is synthesized to contain two chemical compound conjugates, each of which can recruit a protein. In this example, the linear RNA is bound to the E3 ubiquitin ligase VHL and EGFR, thereby targeting EGFR for ubiquitination and degradation.

A linear RNA is designed to include conjugated VH 032 and gefitinib.

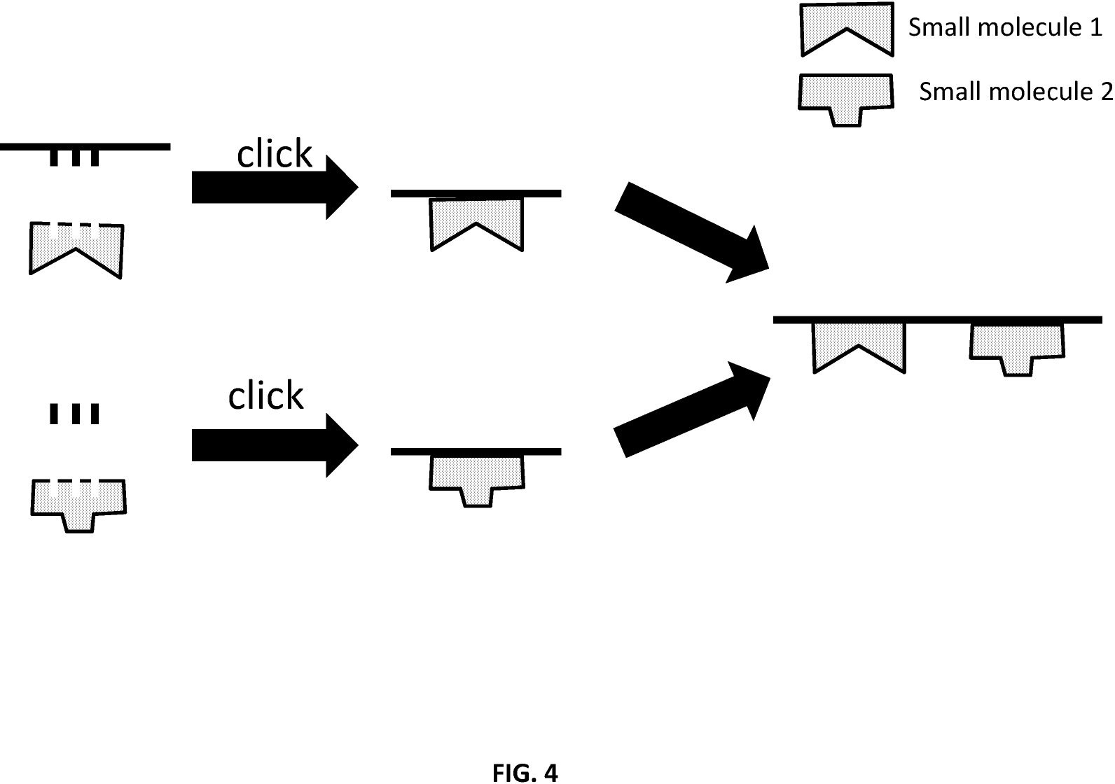

Two linear RNA segments containing molecular handles are separately transcribed and conjugated to VH 032 or gefitinib. The two linear RNA segments are then ligated together as illustrated in FIG. 4.

A first linear RNA comprising a molecular handle is synthesized either by in vitro transcription using T7 RNA polymerase (Lucigen) or custom-synthesized by a commercial entity. UTP is substituted with 5-azido-C3-UTP (Jena Biosciences) during the in vitro transcription reaction to generate azide-functionalized RNA. Synthesized linear RNA can comprise unmodified or modified bases. Synthesized linear RNA is purified with RNA clean up kit (New England Biolabs). Alkyne-functionalized VH 032 is conjugated to the azide-functionalized segment of the linear RNA via Copper-catalyzed Azide-Alkyne click chemistry reactions (CuAAC) with the CuAAC Biomolecule Reaction Buffer Kit based on manufacturer’s instructions (Jena Bioscience). VH 032-conjugated linear RNA is purified with an RNA clean up kit (New England Biolab).

A second linear RNA comprising a molecular handle is synthesized either by in vitro transcription using T7 RNA polymerase (Lucigen) or custom-sythesized by a commercial entity. UTP is substituted with 5-azido-C3-UTP (Jena Biosciences) during the in vitro transcription reaction to generate azide-functionalized RNA. Synthesized linear RNA can comprise unmodified or modified bases. Synthesized linear RNA is purified with RNA clean up kit (New England Biolabs). Alkyne-functionalized gefitinib is conjugated to the azide-functionalized segment of the linear RNA via Copper-catalyzed Azide-Alkyne click chemistry reactions (CuAAC) with the CuAAC Biomolecule Reaction Buffer Kit based on manufacturer’s instructions (Jena Bioscience). Gefitinib-conjugated linear RNA is purified with an RNA clean up kit (New England Biolab).

The two oligonucleotides are ligated together using the T4 DNA ligase, then subjected to RNA 5′ Pyrophosphohydrolase (RppH, New England Biolabs) treatment to remove pyrophosphate. RppH-treated linear RNA is purified with an RNA clean up kit (New England Biolabs).

Linear RNA binding to VHL is assessed, for example, by pull down of GST-VHL followed by RT-qPCR for linear RNA detection.

Linear RNA binding to EGFR is assessed, for example, by pull down of poly-histidine tagged EGFR followed by RT-qPCR for linear RNA detection.

Linear RNA binding to VHL and EGFR is assessed, for example, by pull down of GST-VHL followed by Western Blot for EGFR, or by pull-down of poly-histidine tagged EGFR followed by Western Blot for VHL.

Degradation of EGFR is quantified by, for example, Western Blot or Enzyme Linked Immunosorbent Assay (ELISA) after delivering the linear RNA to cells or an in vitro system comprising EGFR and components of the E3 ubiquitin ligase and proteasomal degradation pathway.

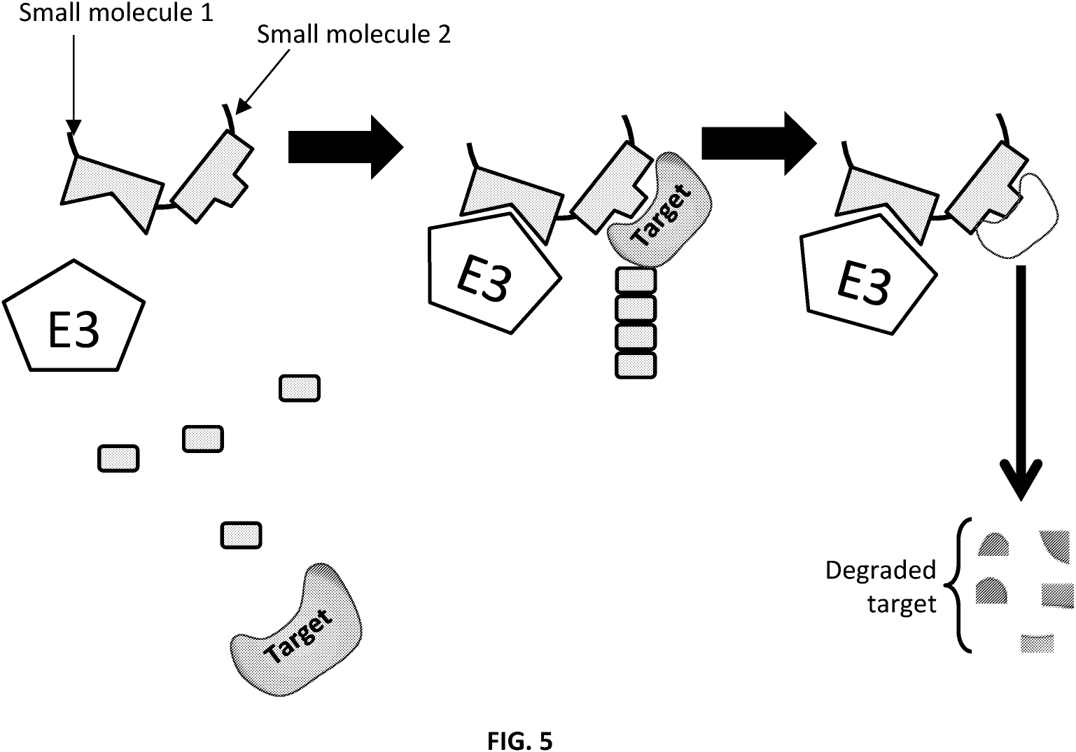

FIG. 5 illustrates a linear RNA with two chemical compound conjugates that can target a protein for ubiquitination and degradation.

### Example 4: Linear RNA That Binds Two Proteins

This Example describes linear RNA simultaneously binding to two proteins.

The E3 ubiquitin ligase, MDM2, binds and ubiquitinates proteins, such as p53, marking them for degradation by the proteasome. The following example shows that linear RNA is simultaneously bound to MDM2 and p53. The MDM2-dependent ubiquitination of p53 is enhanced by this binding.

Linear RNA is designed to include the sequence of FOX3 RNA that binds MDM2 and p53.

Unmodified linear RNA is synthesized either by in vitro transcription using T7 RNA polymerase from a DNA segment having the appropriate sequence or custom-synthesized by a commercial entity. Synthesized linear RNA can comprise unmodified or modified bases. Transcribed RNA is purified with an RNA purification system (QIAGEN), treated with alkaline phosphatase (ThermoFisher Scientific, EF0652) following the manufacturer’s instructions, and purified again with the RNA purification system.

Linear RNA binding to MDM2 and p53 is assessed by an electrophoretic mobility shift assay to visualize each RNA-protein complex or alternatively by pull-down of linear RNA using a biotinylated oligo complementary to a region of the linear RNA followed by immunoblotting. Additionally, MDM2 ubiquitination of p53 through binding of linear RNA is assayed via immunoblotting with anti-ubiquitin antibodies or by mass-spectrometry. Degradation of p53 protein can be quantified by, for example, Western Blot or Enzyme Linked Immunosorbent Assay (ELISA).

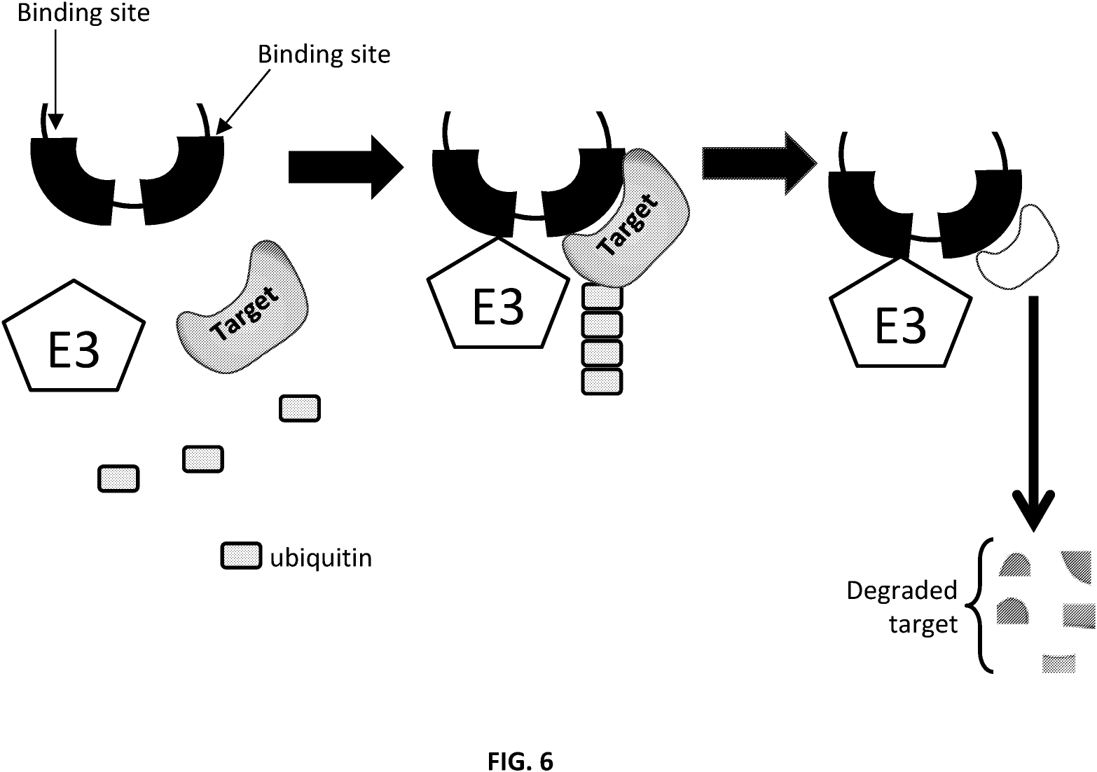

FIG. 6 illustrates a linear RNA that can bind two proteins and target one of the proteins for ubiquitination and degradation.

### Example 5: Linear RNA That Binds Protein

This Example describes linear RNA comprising a protein binding site binds to protein.

Human antigen receptor (HuR) can be a pathogenic protein, e.g. it is known to bind and stabilize cancer related mRNA transcripts, such as mRNAs for proto-oncogenes, cytokines, growth factors, and invasion factors. HuR has a central tumorigenic activity by enabling multiple cancer phenotypes. Sequestration of HuR with linear RNA may attenuate tumorigenic growth in multiple cancers. The following example shows a linear RNA is bound to HuR for sequestration.

Linear RNA is designed to include the HuR RNA binding motifs: 5′-UCAUAAUCAA UUUAUUAUUUUCUUUUAUUUUA UUCACAUAAUUUUGUUUUU-3′, 5′-AUUUUGUUUUUAA CAUUUC-3′, 5′-UCAUAAUCAAUUUAUUAUUUUCUUUUAUUUUAUUCACAUAAUUUUGUUU UUAUUUUGUUUUUAACAUUUC-3′ to competitively bind HuR and inhibit its binding/downstream functions.

Linear RNA is synthesized either by in vitro transcription using T7 RNA polymerase from a DNA segment comprising the HuR RNA motif and protein binding sequence or custom-synthesized by a commercial entity. Synthesized linear RNA can comprise unmodified or modified bases.

Transcribed RNA is purified with a Monarch RNA cleanup kit (New England Biolabs, T2050), treated with RNA 5′-phosphohydrolase (RppH, New England Biolabs, M0356) following the manufacturer’s instructions, and purified again with the RNA purification column. RNA quality is assessed by Urea-PAGE or through automated electrophoresis (Agilent).

Linear RNA binding to HuR is evaluated in vitro by RNA immunoprecipitation (RIP) for HuR. HuR protein is bound to linear RNAs containing the HuR RNA-binding motif, while no binding above background is detected by the linear RNAs lacking the HuR RNA-binding motif.

Therefore, a biomolecule of therapeutic interest selective is selectively bound to linear RNA.

### Example 6: Linear RNA With Two Different Chemical Compounds

This Example describes linear RNA linked to chemical compounds that recruits two different proteins of choice.

Thalidomide, a clinically approved drug (Thalomid), is known to associate with a member of the cells’ protein degradation machinery, the E3 ubiquitin ligase. By conjugating thalidomide to linear RNA (e.g., via click chemistry), thalidomide-conjugated linear RNA can recruit cells’ degradation machinery to a second, disease-causing protein (e.g., also targeted by the linear RNA). The following Example describes two chemical compounds (thalidomide and JQ1) that are conjugated to a linear RNA are bound to (1) E3 ubiquitin ligase Cereblon for ubiquitination and subsequent degradation of a neighboring protein and (2) BET family proteins through JQ1, which is a chemical compound inhibitor that binds to BET family proteins.

LinearRNA is designed to include reactive uridine residues (e.g., 5-azido-C3-UTP) for conjugation of alkyne-functionalized small molecules, known to interact with an intracellular protein of interest.

Linear RNA is synthesized either by in vitro transcription using T7 RNA polymerase (Lucigen) or custom-synthesized by a commercial entity. All UTPs are substituted with 5-azido-C3-UTP (Jena Biosciences) in the in vitro transcription reaction to generate azide-functionalized RNA. Synthesized linear RNA can comprise unmodified or modified bases. Synthesized linear RNA is purified with an RNA clean up kit (New England Biolabs) and is subjected to RNA 5′ Pyrophosphohydrolase (RppH, New England Biolabs) treatment to remove pyrophosphate. RppH-treated linear RNA is purified with an RNA clean up kit (New England Biolabs).

Alkyne-functionalized thalidomide and alkyne-functionalized JQ1 (Jena Bioscience) is conjugated to azide-functionalized linear RNA via Copper-catalyzed Azide-Alkyne click chemistry reactions (CuAAC) with the click chemistry reaction kit based on manufacturer’s instructions (Jena Bioscience). For comparison, three different kinds of chemical compound conjugated linear RNA are generated; RNA with both JQ1 and thalidomide, thalidomide only, JQ1 only. Chemical compound-conjugated linear RNA is purified with an RNA clean up kit (New England Biolab).

Chemical compound-conjugated linear RNA binding to E3 ubiquitin ligase CRBN and BET famiy proteins is analyzed using GST pull-down. GST-CRBN (Abcam) and one of the BET family protein, Bromodomain containing protein 4 (BRD4, BPSBiosciences) is used for this experiment. For GST pull-down assay, thalidomide and JQ1 conjugated-linear RNA (2 nM) iss incubated with GST-CRBN and BRD4 (50 nM each) for 2 hours at room temperature in the presence of 25 mM Tris-Cl (pH 7.0), 100 mM NaCl, 1 mM EDTA, 0.5% NP-40, 5% Glycerol. Azide-functionalized linear RNA without conjugation, thalidomide conjugated RNA, and JQ1 conjugated RNA is used as negative controls. RNA-protein mixture is further incubated with GSH-agarose bead to allow GST-GSH interaction for an hour at room temperature. After washing three times with binding buffer, the bead is separated to two equal parts. To monitor protein binding, one part of the bead is boiled in the presence of Lammli Sample Buffer (Bio-Rad) and is subjected to western blot with BRD4 antibody (for detecting BRD4 protein) and GST antibody (for detecting GST-CRBN). To monitor RNA recruitment, the RNA on the bead is extracted with Trizol (Thermo Fisher) and the extracted linear RNA is reverse transcribed and is detected by quantitative RT-PCR with primers specific for linear form of RNA.

It is expected that linear RNA containing the thalidomide and JQ1 small molecules is highly enriched in the GST pull down for both CRBN as well as BET domain protein BRD4, demonstrating that not only can linear RNA contain a chemical compound, but it can bind to two specific proteins using this chemical compound conjugate to degrade the protein of choice.

What is claimed is:

1-39. (canceled)

40. A complex comprising a linear polyribonucleotide bound to two or more copies of a target protein and to two or more copies of a substrate protein, wherein the target protein modulates the substrate protein thereby promoting degradation of the substrate protein.

41. The complex of claim 40, wherein the linear polyribonucleotide is bound to five or more copies of a target protein.

42. The complex of claim 41, wherein the linear polyribonucleotide is bound to ten or more copies of the target protein.

43. The complex of claim 40, wherein the linear polyribonucleotide is bound to five or more copies of the substrate protein.

44. The complex of claim 43, wherein the linear polyribonucleotide is bound to ten or more copies of the substrate protein.

45. The complex of claim 40, wherein the target protein is an enzyme.

46. The complex of claim 45, wherein the enzyme is a post-translational modification enzyme.

47. The complex of claim 46, wherein the post-translational modification enzyme modifies the substrate by adding a functional group to the substrate protein.

48. The complex of claim 40, wherein the target protein is a ubiquitin ligase.

49. The complex of claim 48, wherein the ubiquitin ligase ubiquitinates the substrate protein.

50. The complex of claim 48, wherein the ubiquitin ligase is an E3 ubiquitin ligase.

51. The complex of claim 40, wherein the linear polyribonucleotide comprises two or more modified nucleotides each comprising a functional group conjugated to a first chemical compound, wherein the first chemical compound binds the substrate protein.

52. The complex of claim 40, wherein the linear polyribonucleotide comprises two or more modified nucleotides each comprising a functional group conjugated to a second chemical compound, wherein the second chemical compound binds the target protein.

53. The complex of claim 40, wherein the linear polyribonucleotide comprises two or more binding moieties, wherein each binding moiety binds to the substrate protein.

54. The complex of claim 40, wherein the linear polyribonucleotide comprises two or more binding moieties, wherein each binding moiety binds to the target.

55. The complex of claim 40, wherein, the linear polyribonucleotide comprises two or more modified nucleosides nucleotides each comprising a functional group conjugated to a first chemical compound, wherein the first chemical compound binds the substrate protein; and wherein the linear polyribonucleotide comprises two or more modified nucleosides each comprising a functional group conjugated to a second chemical compound, wherein the second chemical compound binds the target protein.

56. The complex of claim 40, wherein, the linear polyribonucleotide comprises two or more modified nucleosides each comprising a functional group conjugated to a first chemical compound, wherein the first chemical compound binds the substrate protein; and wherein the linear polyribonucleotide comprises two or more binding moieties, wherein each binding moiety binds to the target.

57. A pharmaceutical composition comprising the complex according to claim 40.

58. A linear polyribonucleotide capable of binding to two or more copies of a target protein and to two or more copies of a substrate protein for use in promoting degradation of the substrate protein, wherein the linear polyribonucleotide contacts the target protein and the substrate protein, further wherein the target protein modulates the substrate protein thereby promoting degradation of the substrate protein.

59. A method of promoting degradation of a substrate protein, the method comprising providing a linear polyribonucleotide capable of binding to two or more copies of a target protein and to two or more copies of the substrate protein, to contact the linear polyribonucleotide with the target protein and the substrate protein, further wherein the target protein modulates the substrate protein thereby promoting degradation of the substrate protein.
